(19) United States
(12) Patent Application Publication
(10) Pub. No.: US 2026/0209684 A1
(43) Pub. Date: Jul. 23, 2026

(54) RECOMBINANT FUNGAL STRAINS AND METHODS THEREOF FOR PRODUCING CONSISTENT PROTEINS

(71) Applicant: DANISCO US INC., Palo Alto, CA (US)

(72) Inventors: Lori Ann Maggio-Hall, Jeannette, PA (US); Timothy Caspar, Yorklyn, DE (US); Carol M. McCutchen, Avondale, PA (US); Seung-Pyo Hong, Hochessin, DE (US); Suzanne Koch Singles, Newark, DE (US); George C. Fox, Wilmington, DE (US); Sergio C. Nanita, Media, PA (US)

(21) Appl. No.: 19/140,278

(22) PCT Filed: Dec. 14, 2023
(86) PCT No.: PCT/US2023/084137

§ 371 Date: Jun. 17, 2025

Related U.S. Application Data
(60) Provisional application No. 63/476,079, filed on Dec. 19, 2022.

(51) Int. Cl. C12N 1/14; C12N 9/24; C12P 21/00; C12R 1/885

(52) U.S. Cl. CPC C12N 1/14; C12N 9/2402; C12N 9/2488; C12P 21/00; C12R 2001/885; C12Y 302/0102; C12Y 302/01024

(57) ABSTRACT

Certain one or more embodiments of the instant disclosure are related to, inter alia, recombinant (genetically modified) filamentous fungal cells (strains) producing proteins of interest, methods and compositions for the design and construction of modified filamentous fungal cells producing proteins of interest, methods and compositions for the expression/production/secretion/recovery and the like of endogenous and/or heterologous proteins of interest (e.g., phytases, lipases, glucoamylases, phospholipases, esterases, cellulases, hemicellulases, xylanases, etc.) in modified filamentous fungal cells, methods and compositions for producing proteins of interest in recombinant filamentous fungal, wherein the proteins produced and secreted into the broth and/or proteins recovered from the broth have uniform and consistent N-linked glycosylation patterns, and/or reduced (unwanted) glycation of one or more proteins of interest, and the like.

### FIELD

The present disclosure is generally related to the fields of biology, molecular biology, filamentous fungi, fermentation, genetics, glycoproteins, industrial proteins, protein production and the like. More particularly, the present strains, compositions and methods of the disclosure relate to genetic modifications in filamentous fungi that give rise to recombinant (modified) strains having altered phenotypes, wherein such recombinant strains are particularly well-suited for growth in submerged cultures (e.g., large-scale production of proteins for industrial/commercial applications).

### CROSS REFERENCE TO RELATED APPLICATIONS

This application claims benefit to U.S. Provisional Patent Application No. 63/476,079, filed Dec. 19, 2022, which is incorporated herein by referenced in its entirety.

### REFERENCE TO A SEQUENCE LISTING

The contents of the electronic submission of the text file Sequence Listing, named “NB42034-WO-PCT_SequenceListing.txt” was created on Oct. 24, 2023 and is 68 KB in size, which is hereby incorporated by reference in its entirety.

### BACKGROUND

Filamentous fungi (e.g., Aspergillus sp., Penicillium sp., Talaromyces sp., Fusarium sp., Myceliophthora sp., Neurospora sp., Candida sp., Trichoderma sp., and the like) are capable of expressing native and heterologous proteins to high levels, making them well-suited for the large-scale production of proteins (e.g., enzymes, antibodies, receptors, peptides, etc.) and/or metabolites for industrial and commercial applications such as pharmaceutical applications, animal health applications, food applications, beverage applications, laundry and textile applications, and the like. Filamentous fungi are typically grown in mycelial submerged cultures in bioreactors (fermentors), which bioreactors are adapted to introduce and distribute oxygen and nutrients into the culture medium (i.e., culture broth). For example, the filamentous fungus Trichoderma reesei (T. reesei; an anamorph of the fungus Hypocrea jecorina) is known to be an efficient producer of cellulase enzymes.

As such, filamentous fungi have been utilized for their ability to produce proteins (e.g., enzymes), which proteins are valuable in the production of commodities such as cellulosic (derived) ethanol, textile processing, grain processing, detergents, fibers/pulp/paper, food additives, feed additives, and the like. Likewise, filamentous fungi are utilized for their ability to produce protein biologics (e.g., antibodies, antibody fragments, protein receptors, growth factors, and the like). For example, recombinant gene expression in such fungal host strains is a common method for production of proteins and as such, protein productivity improvements of a fungal host strain are an important economic factor of protein production costs. In certain embodiments, proteins produced by filamentous fungi may contain post-translational modifications, including protein glycosylations at asparagine, threonine and/or serine residues, wherein proteins comprising such glycosylations are referred to as glycoproteins. In particular, the glycosylation process consists of a number of biochemical modifications commensurate with the passage of the target glycoprotein along the secretory pathway. While this process is well-regulated, the final extent of protein glycosylation depends on many factors, including structural characteristics of the glycoprotein itself, growth conditions and the like.

As appreciated by one of skill in the art, the recombinant expression of heterologous glycoproteins in filamentous fungal strains can be problematic, often resulting in glycoprotein products of less than satisfactory quality. For example, glycoproteins of less than satisfactory quality include, glycoproteins having non-uniform (inconsistent) glycosylation patterns which can add significant costs to downstream processing (e.g., recovery and purification of the glycoprotein product), glycoproteins having reduced activity, glycoproteins having reduced stability, and the like. In other embodiments, glycation (i.e., non-enzymatic glycosylation) of recombinant proteins produced in fungal strains can be problematic, often resulting in recombinant protein products of less than satisfactory quality. For example, the glycation (non-enzymatic glycosylation) of proteins (e.g., enzymes) can be a substantial issue, as it often reduces the activity of the protein (enzyme) product (Sutthirak et al., 2005). Additionally, during recovery processes for many proteins, the clarified (fermentation) broth must generally be diafiltered to remove any reactive free sugars in order to limit glycation. Subsequent steps in the recovery of the protein (e.g., heat treatments to remove any (unwanted) background enzymatic activities) may cause more free sugars to be released from the material.

Thus, as appreciated by one of skill in the art, novel compositions and methods for the enhanced production of recombinant proteins in filamentous fungal strains are of significant commercial interest. In certain embodiments, there remain ongoing and unmet needs in the art related to, inter alia, the recombinant expression of heterologous proteins in fungal strains, the recombinant expression of heterologous glycoproteins in fungal strains, and the like, wherein the recombinant proteins produced have uniform (consistent) glycosylation patterns, reduced glycation (i.e., non-enzymatic glycosylation), reduced downstream processing requirements, and the like.

### SUMMARY

As generally set forth and described hereinafter, certain embodiments of the disclosure are related to, inter alia, recombinant filamentous fungal cells (strains) producing proteins of interest, methods and compositions for the design and construction of recombinant (modified) fungal cells producing proteins of interest, methods and compositions for the expression/production of endogenous proteins of interest (e.g., lignocellulosic degrading enzymes and the like) in recombinant filamentous fungal cells, methods and compositions for the expression/production of heterologous proteins of interest (e.g., phytases, lipases, glucoamylases, phospholipases, esterases, cellulases, hemicellulases, xylanases, etc.) in recombinant filamentous fungal cells, methods and compositions for producing proteins of interest in recombinant filamentous fungal, wherein the proteins produced and/or recovered therefrom have uniform and consistent N-linked glycosylation patterns, and/or reduced (unwanted) protein of interest glycation events, and the like. Thus, in certain embodiments, the methods and compositions set forth and described herein provide, inter alia, significant cost savings, as related to large scale fermentation and downstream recovery one or more proteins of interest produced in one or more recombinant filamentous fungal cells described herein.

### BRIEF DESCRIPTION OF THE BIOLOGICAL SEQUENCES

SEQ ID NO: 1 is a nucleic acid (DNA) sequence of the wild-type Trichoderma mds1 gene encoding the native Mds1 protein of SEQ ID NO: 2.

SEQ ID NO: 2 is the amino acid sequence of the native Mds1 protein encoded by SEQ ID NO: 1.

SEQ ID NO: 3 is a DNA sequence of the wild-type Trichoderma mds2 gene encoding the native Mds2 protein of SEQ ID NO: 4.

SEQ ID NO: 4 is the amino acid sequence of the native Mds2 protein encoded by SEQ ID NO: 3.

SEQ ID NO: 5 is a DNA sequence of the wild-type Trichoderma gls2a gene encoding the native GIIα protein of SEQ ID NO: 6.

SEQ ID NO: 6 is the amino acid sequence of the native GIIα protein encoded by SEQ ID NO: 5.

SEQ ID NO: 7 is a mutant Trichoderma DNA sequence (gls2a^{Stop}) encoding a variant (truncated) GIIα^{Stop} protein.

SEQ ID NO: 8 is the amino acid sequence of the truncated GIIα^{Stop} variant protein encoded by SEQ ID NO: 7.

SEQ ID NO: 9 is a synthetic RNA sequence named RGH2.

SEQ ID NO: 10 is a synthetic DNA gls2a restoration donor sequence.

SEQ ID NO: 11 is a synthetic RNA sequence named LFP009.

SEQ ID NO: 12 is a synthetic RNA sequence named LFP010.

SEQ ID NO: 13 is a synthetic DNA sequence named LFP013.

SEQ ID NO: 14 is a synthetic DNA sequence named LFP014.

SEQ ID NO: 15 is a synthetic RNA sequence named TCg3.

SEQ ID NO: 16 is a synthetic RNA sequence named TCg4.

SEQ ID NO: 17 is a synthetic DNA sequence named TC128.

SEQ ID NO: 18 is the DNA sequence of an Aspergillus niger gls2a gene homolog.

SEQ ID NO: 19 is the amino acid sequence of the A. niger GIIα protein encoded by SEQ ID NO: 18.

SEQ ID NO: 20 is the DNA sequence of an A. niger mds1 gene homolog.

SEQ ID NO: 21 is the amino acid sequence of the A. niger Mds1 protein encoded by SEQ ID NO: 20.

SEQ ID NO: 22 is the DNA sequence of an A. niger mds2 gene homolog.

SEQ ID NO: 23 is the amino acid sequence of the A. niger Mds2 protein encoded by SEQ ID NO: 22.

SEQ ID NO: 24 is the DNA sequence of a T. thermophilus gls2a gene homolog.

SEQ ID NO: 25 is the amino acid sequence of the T. thermophilus GIIα protein encoded by SEQ ID NO: 24.

SEQ ID NO: 26 is the DNA sequence of a T. thermophilus mds1 gene homolog.

SEQ ID NO: 27 is the amino acid sequence of the T. thermophilus Mds1 protein encoded by SEQ ID NO: 26.

SEQ ID NO: 28 is the DNA sequence of a T. thermophilus mds2 gene homolog.

SEQ ID NO: 29 is the amino acid sequence of the T. thermophilus Mds2 protein encoded by SEQ ID NO: 28.

SEQ ID NO: 30 is an artificial RNA sequence named LFP028.

SEQ ID NO: 31 is an artificial RNA sequence named LFP029.

SEQ ID NO: 32 is an artificial DNA sequence named LFP030.

SEQ ID NO: 33 is an artificial RNA sequence named LFP031.

SEQ ID NO: 34 is an artificial RNA sequence named LFP032.

SEQ ID NO: 35 is an artificial DNA sequence named LFP033.

SEQ ID NO: 36 is the DNA sequence of wild-type T. reesei Endo T allele encoding a native Endo-N-acetyl-β-D-glucosaminidase (ENGase) protein.

### BRIEF DESCRIPTION OF DRAWINGS

FIG. 1 presents the amino acid sequences of the native Trichoderma Mds1 protein (FIG. 1A, SEQ ID NO: 2) and the native Trichoderma Mds2 protein (FIG. 18. SEQ ID NO: 4).

FIG. 2 presents the amino acid sequences of the native GIIα protein (FIG. 2A, SEQ ID NO: 6) and the truncated GIIα^{Stop} variant protein (FIG. 2B, SEQ ID NO: 8) encoded by the mutant gls2a^{STOP} allele (SEQ ID NO: 7). As shown in FIG. 2A, the native GIIα protein (SEQ ID NO: 6) comprises 964 amino acid residues, wherein the last 310 C-terminal residues of the native protein are underlined. As shown in FIG. 2B, the variant (truncated) GIIα^{Stop} protein (SEQ ID NO: 8) comprises 807 amino acid residues, wherein the truncated C-terminus comprises 153 (frameshifted) amino acid residues are underlined.

FIG. 3 shows an SDS PAGE analysis of diluted phytase produced in MTP cultures. The effect of restoration of gls2a (gls2a^{R}) allele confirmed in three (3) independent isolates (Phy-gls2a^{R}) versus two (2) technical replicates of the (Phy) parent strain (Parent) is shown. As presented in FIG. 3, the left panel shows the phytase (Phy) parent and modified gls2a restored (gls2a^{R}) strain with no Endo H treatment and the right panel shows the same phytase (Phy) parent and modified gls2a restored (gls2a^{R}) strain with EndoH treatment.

FIG. 4 shows electrospray ionization mass spectra (EIMS) of phytase produced from Phy strain before (Control) and after (gls2a^{R}) restoration of the truncated gls2a^{STOP} allele. Data presented are from supernatant samples obtained from large scale fermenters at one-hundred forty-eight (148) hour time points. Protein was detected at multiple charge states as indicated.

FIG. 5 presents the amino acid sequences of the T. reesei GIIα protein (SEQ ID NO: 6), an A. niger GIIα protein homologue (SEQ ID NO: 19) and a T. thermophilus GIIα protein homologue (SEQ ID NO: 25).

FIG. 6 shows a CLUSTAL multiple sequence alignment of the T. reesei GIIα protein (SEQ ID NO: 6, labeled “6”), A. niger GIIα homologue (SEQ ID NO: 19, labeled “19”) and T. thermophilus GIIα homologue (SEQ ID NO: 25, labeled “25”).

FIG. 7 presents the amino acid sequences of the T. reesei Mds1 protein (SEQ ID NO: 2), an A. niger Mds1 protein homologue (SEQ ID NO: 21) and a T. thermophilus Mds1 protein homologue (SEQ ID NO: 27).

FIG. 8 shows a CLUSTAL multiple sequence alignment of the T. reesei Mds1 protein (SEQ ID NO: 2, labeled “2”), A. niger Mds1 homologue (SEQ ID NO: 21, labeled “21”) and T. thermophilus Mds1 homologue (SEQ ID NO: 27, labeled “27”).

FIG. 9 presents the amino acid sequences of the T. reesei Mds2 protein (SEQ ID NO: 4), an A. niger Mds2 protein homologue (SEQ ID NO: 23) and a T. thermophilus Mds2 protein homologue (SEQ ID NO: 29).

FIG. 10 shows a CLUSTAL multiple sequence alignment of the T. reesei Mds2 protein (SEQ ID NO: 4 labeled “4”), A. niger Mds2 homologue (SEQ ID NO: 23, labeled “23”) and T. thermophilus Mds2 homologue (SEQ ID NO: 29, labeled “29”).

FIG. 11 presents annotated amino acid sequence positions of the native T. reesei Mds1 (SEQ ID NO: 2) and Mds2 (SEQ ID NO: 4) proteins. In particular, as shown in FIG. 11, the Mds1 protein comprises 523 amino acid residues, wherein amino acid residues from about position 43 to about position 511 (SEQ ID NO: 2) are indicated with bold residues, wherein these amino acid positions (~43 to ~511) comprise a glycosyl hydrolase family 47 (GH47) sequence domain. Likewise, as shown in FIG. 11, the Mds2 protein comprises 794 amino acid residues, wherein amino acid residues from about position 39 to about position 286 (SEQ ID NO: 4) are indicated with underlined residues, and amino acid residues positions from about position 292 to about position 773 (SEQ ID NO: 4) are indicated with bold residues. In particular, amino acid positions of about position 39 to 286 comprise an N-terminal glycosyl hydrolase family 92 (GH92) sequence domain, and amino acid positions of about position 292 to 773 comprise a glycosyl hydrolase family 92 (GH92; super-family) sequence domain.

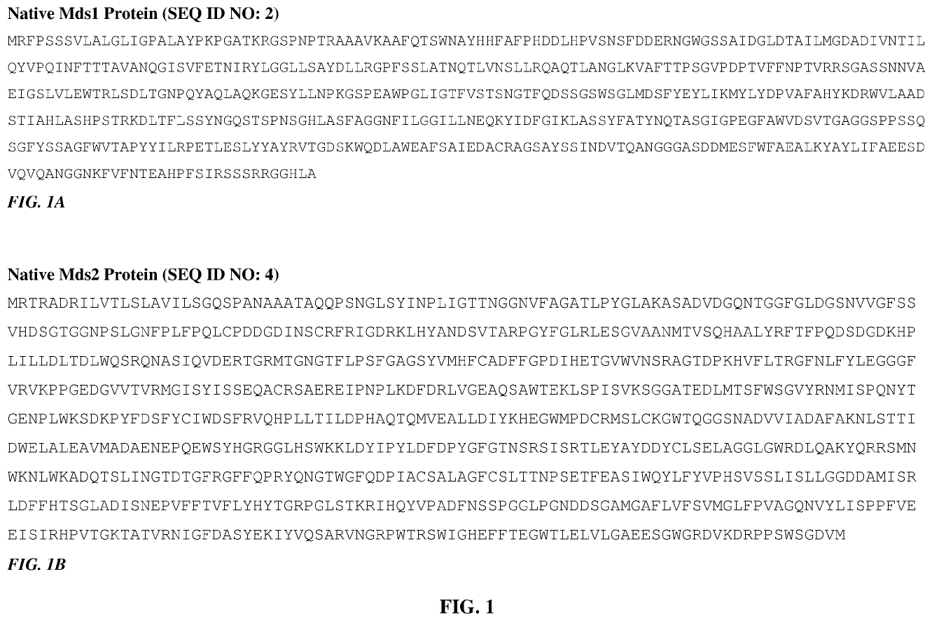

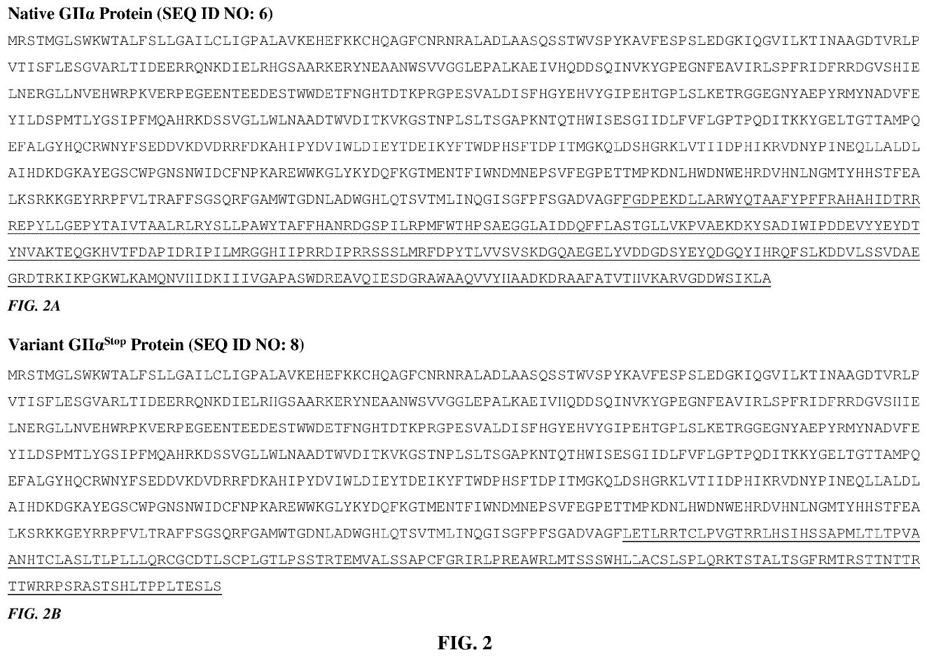

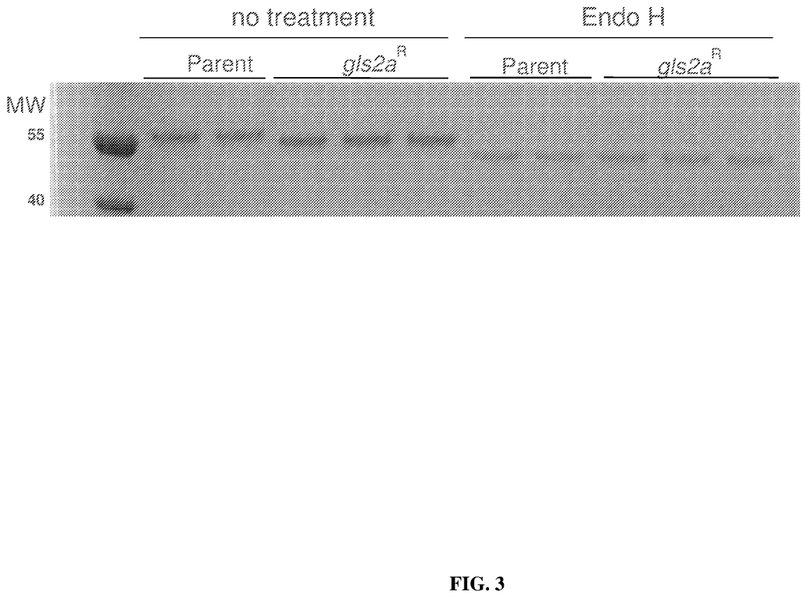

### DETAILED DESCRIPTION

As described herein, certain embodiments are related to recombinant (modified) filamentous fungal cells (strains) for use in the commercial scale production of proteins (polypeptides) of interest. More particularly, the present strains and methods of the disclosure relate to genetic modifications in filamentous fungi that give rise to recombinant (modified) strains having altered phenotypes, wherein such variant strains are particularly well-suited for growth in submerged cultures (e.g., large-scale production of proteins for industrial/commercial applications). In certain embodiments, the disclosure provides, inter alia, recombinant filamentous fungal cells (strains) producing proteins of interest, methods and compositions for the expression/production of endogenous and/or heterologous proteins of interest in recombinant filamentous fungal cells, methods and compositions for producing proteins of interest in recombinant filamentous fungal, wherein the proteins produced and recovered therefrom have uniform and consistent N-linked glycosylation patterns, and/or reduced (unwanted) protein glycation, and/or enhanced storage stability and/or enhanced protein (enzyme) activity and the like.

### I. Definitions

Prior to describing the present strains, compositions and methods in further detail, the following terms and phrases are defined. Terms not defined should be accorded their ordinary meaning as used and known to one skilled in the art.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation or “proviso”. For example, in certain embodiments, the proviso “wherein the medium does not comprise an inducing substrate” may be used to exclude inducing substrates such as cellulose, lactose, gentibiose, sophorose and the like.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the term “Ascomycete fungal cell” refers to any organism in the Division Ascomycota in the Kingdom Fungi. Examples of Ascomycetes fungal cells include, but are not limited to, filamentous fungi in the subphylum Pezizomycotina, such as Trichoderma sp., Aspergillus sp., Myceliophthora sp., Penicillium sp., and the like.

As used herein, the term “filamentous fungus” refers to all filamentous forms of the subdivision Eumycota and Oomycota. For example, filamentous fungi include, without limitation, Acremonium, Aspergillus, Emericella, Fusarium, Humicola, Mucor, Myceliophthora, Neurospora, Penicillium, Scytalidium, Thielavia, Tolypocladium, and Trichoderma species.

In certain embodiments, a filamentous fungus is a Trichoderma sp. cell (strain) including, but not limited to, Trichoderma harzianum, Trichoderma koningii, Trichoderma longibrachiatum, Trichoderma reesei, Trichoderma viride and the like. As known to one skilled in the art. Trichoderma reesei was previously classified as “Hypocrea jecorina”. Exemplary parental Trichoderma reesei strains include, but are not limited to, T. reesei strain QM6a (ATCC® 13631), T. reesei strain RL-P37 (NRRL Deposit No. 15709) and T. reesei strain RUT-C30 (ATCC® 56765). For example, Trichoderma strains Rut-C30 and RL-P37 are mutagenized derivatives of T. reesei natural isolate QM6a (Le Crom et al., 2009; Sheir-Neiss and Montenecourt, 1984), with strain NG14 being the last common ancestor. Thus, in certain embodiments, an exemplary filamentous fungal strain may be derived/obtained from T. reesei strain RL-P37 which may comprise a deletion (A) or a loss of function variant of the T. reesei pvr2 gene (abbreviated hereinafter. “Δpvr2”), as generally described by Sheir-Neiss and Montenecourt (1984) and PCT Publication No. WO2011/153449 (each incorporated herein by reference in its entirety).

In certain embodiments, a filamentous fungus is an Aspergillus sp. cell (strain) such as Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus oryzae. Aspergillus terreus and the like. Exemplary parental Aspergillus sp. strains include, but are not limited to, A. niger strain ATCC® 1015, A. oryzae strain RIB40 (ATCC® 42149), and the like.

In other embodiments, a filamentous fungus is Myceliophthora sp. cell (strain) such as Myceliophthora thermophila (also known as, Thermothelomyces thermophilus) and the like. Exemplary parental Myceliophthora sp. strains include, but are not limited to, M. thermophila strain ATCC® 42464.

In certain other embodiments, fungal strains constructed herewith utilize an Aspergillus nidulans amdS (acetamide) gene marker for selection. More specifically, the amdS gene and variants thereof are generally known to one skilled in the art, such as described in PCT Publication No. WO2006/040358 (incorporated herein by reference in its entirety). However, one skilled in the art is not limited and may choose any selection marker (e.g., auxotrophic markers, antibiotic resistance markers) functional in the recombinant microbial cell of choice.

In certain embodiments, recombinant fungal strains of the disclosure have been designed/constructed to express heterologous reporter proteins. In certain embodiments, heterologous reporter proteins include, but are not limited to, recombinant phytase (reporter) proteins, recombinant lipase (reporter) proteins, recombinant glucoamylase (reporter) proteins and the like.

In certain embodiments, exemplary phytase (reporter) proteins include, but are not limited to, native or engineered (variant) phytases. For example, PCT Publication No. WO2003/038111 generally describes purified enzymes having phytase activity derived from various filamentous fungal species (e.g., Penicillium, sp., Fusarium sp., Humicola sp., Emericella sp.) and suitable methods for constructing recombinant fungal strains expressing/producing such phytase proteins and methods for assaying the same. Likewise, PCT Publication Nos. WO2008/097619, WO2009/129489 and WO2013/119470 (each incorporated herein by reference in its entirety) describe engineered (variant) Buttiauxella sp. phytases and related methods for constructing fungal strains expressing/producing such variant phytases and methods for assaying the same.

In certain other embodiments, exemplary glucoamylase (reporter) proteins include, but are not limited to native or engineered (variant) glucoamylases. For example, PCT Publication Nos. WO2008/04589, WO2009/067218, WO2011/020852 and WO2021/212095 generally describe methods for constructing fungal strains expressing/producing glucoamylases and methods for assaying the same.

In certain other embodiments, exemplary lipase (reporter) proteins include, but are not limited to native or engineered (variant) lipases. For example, PCT Publication No. WO2020/190782 generally describes methods for constructing fungal strains expressing/producing lipases and methods for assaying the same.

In certain other one or more embodiments, recombinant fungal strains of the disclosure are designed, constructed, fermented and the like for the expression and secretion one or more heterologous proteins of interest described below in Section IV.

In certain embodiments, phytase (reporter) proteins, lipase (reporter) proteins and/or glucoamylase (reporter) proteins are referred to as exemplary “proteins of interest”.

As used herein, the term “glycoprotein” may refer to a protein of interest comprising one or more oligosaccharide (carbohydrate/glycan) chains covalently attached via glycoside linkage(s) to one or more amino acid (residue) sidechains (of the glycoprotein). In other embodiments, the term “glycoprotein” may refer to a protein which contained an oligosaccharide side chain, but later has lost it. For example, as understood by one of skill in the art, covalently attached oligosaccharide (glycan) chains of glycoproteins may be partially or completely cleaved by cellular enzymes and/or exogenously introduced enzymes.

As generally known for filamentous fungal cells, N-linked glycans are initially derived from a tetradecasaccharide comprised of three monosaccharide building blocks, mannose (Man), glucose (Glc) and N-acetylglucosamine (GlcNAc), comprising a “Glc3Man9GlcNAc2” structure, with the terminal GlcNAc transferred to appropriately positioned asparagine residues on nascent glycoproteins. Processing of the glycan during passage through the secretory pathway typically removes some of the sugar residues and in some cases adds additional sugar residues. For example, partially, or completely processed glycans reported for Trichoderma include GlcMan8GlcNAc2, GlcMan7GlcNAc2, Man8GlcNAc2, Man7GlcNAc2, Man6GlcNAc2 and Man5GlcNAc2. In some cases, removal of almost the entire glycan chain occurs after secretion, producing proteins with only a single GlcNAc residue.

Likewise. O-linked glycans are attached to hydroxyl groups of L-serine or L-threonine, and this also occurs during the secretory process. The composition of O-linked glycans reported for fungi is highly variable and numerous linear and branched configurations of oligosaccharides have been reported (Goto, 2007). The O-linked oligosaccharides may comprise Glc, Man, as well as two conformations of galactose (Gal), denoted Galp and Galf), and additionally contain phosphate or sulfate linkages. For example, the O-glycans reported for Trichoderma reesei include Man3, phospho-Man2, sulfate-Man2, ManGlc, GlcGalp and GlcManGalp.

As used herein, the phrase “high mannose (Man) structures” refers to oligosaccharides having at least six (6) mannosc residucs (Man), e.g., Man6GlcNAc2, Man7GlcNAc2, Man8GlcNAc2, Man9GlcNAc2, GlcMan8GlcNAc2.

As used herein, the phrase “homogenous N-linked glycan pattern” refers to a glycoprotein comprising or consisting of “Man5GlcNAc2” as the predominant N-linked glycan. In certain embodiments of the disclosure, a glycoprotein comprising a homogenous N-linked glycan pattern comprises at least about 75% Man5GlcNAc2 as the predominant N-linked glycan pattern. In other embodiments, a glycoprotein comprising a homogenous N-linked glycan pattern comprises at least 70% to 100% Man5GlcNAc2 as the predominant N-linked glycan pattern.

As used herein, the phrase “monoglucosylated structure” particularly refers to a protein's N-linked glycan, wherein the monoglucosylated structure comprises or consists of a single (1) glucose (Glc) residue, along with variable numbers of mannose (Man), two GlcNAc and optionally other residues (e.g., GlcMan9GlcNAc2).

As used herein, the term “glycation”, as used in phrases such as, “protein glycation”, “recombinant protein glycation, the “glycation of a protein”, the “glycation of a glycoprotein”, and the like, specifically refers to the “non-enzymatic glycosylation” of a protein (glycoprotein) as understood in the art. As described herein, glycation (non-enzymatic glycosylation) of many proteins of interest is a particularly undesirable (unwanted) result. For example, as generally set forth in Vetter and Indurthi (2011), even moderate protein glycation events can cause unwanted protein structural changes.

As used herein, “reducing sugars” (e.g., glucose, mannose) present in a fermentation broth, down-stream protein recovery process, protein preparation, and the like, generally increase the level (amount) of protein glycation.

Thus, as used and described herein, a “higher level of reducing sugars” present in a fermentation broth, down-stream protein recovery process, protein preparation, and the like, generally correlates with higher levels of protein glycation, wherein such increased levels (amounts) of protein glycation are particularly undesirable, as exemplified in Vetter and Indurthi (2011).

As used herein, the terms “mannosidase protein” and “mannosidases” may be used interchangeably, wherein such mannosidases include “alpha-mannosidases” (e.g., alpha-mannosidase-1; Mds1, alpha-mannosidase-2; Mds2) and “glucosidases”.

As used herein, phrases such as a “gene encoding a mannosidase”, a “mannosidase encoding gene” and “mannosidase gene” may be used interchangeably, wherein such genes encoding a mannosidase include genes encoding “alpha-mannosidases” (e.g., Mds1, Mds2) and “glucosidases”.

As used herein, the term “deficient” means a modified, mutant, or recombinant filamentous fungal cell/strain that produces no detectable activity of one or more (several) mannosidase enzymes compared to a parent (control) filamentous fungal cell/strain when cultivated under identical conditions, or, in the alternative, produces preferably at least 25% less, more preferably at least 50% less, even more preferably at least 75% less, and most preferably at least 95% less of one or more (several) mannosidase enzymes than the parent filamentous fungal cell/strain when cultivated under identical conditions. The level of the one or more mannosidases produced by filamentous fungal cells of the disclosure may be determined using methods described herein or known in the art.

As described herein, and further below in the Examples section, various methods and techniques are known and available for constructing one or marc modified strains of the disclosure.

As used herein, a wild-type Trichoderma reesei “alpha-mannosidase-1 gene” (abbreviated hereinafter, “mds1”) comprises the nucleic acid (DNA) sequence set forth in SEQ ID NO: 1, wherein the wild-type mds1 gene encodes the native “alpha-mannosidase-1 protein” (abbreviated hereinafter, “Mds1”) sequence set forth in SEQ ID NO: 2.

As used herein, a wild-type T. reesei “alpha-mannosidase-2 gene” (abbreviated hereinafter, “mds2”) comprises the DNA sequence set forth in SEQ ID NO: 3, wherein the wild-type mds2 gene encodes the native “alpha-mannosidase-2 protein” (abbreviated hereinafter. “Mds2”) sequence set forth in SEQ ID NO: 4.

As used herein, a wild-type T. reesei “glucosidase II alpha subunit” gene (abbreviated hereinafter “gls2a” or “gls2a”) comprises the DNA sequence set forth in SEQ ID NO: 5, wherein the wild-type gls2a gene encodes the native “glucosidase II alpha subunit” protein (abbreviated hereinafter “GIIα”) sequence set forth in SEQ ID NO: 6.

As used herein, a variant T. reesei “glucosidase II alpha subunit” (gls2a) gene comprising a frameshift mutation at nucleotide position 1,965 (SEQ ID NO: 5) is referred to as “gls2a^{Stop}” (abbreviated hereinafter, “gls2a*w”), wherein the DNA sequence of the gls2a^{Stop} allele is set forth in SEQ ID NO: 7, thereby encoding a truncated GIIα protein. For example, the mutant gls2α^{Stop} gene (nucleotide position 1,965 of SEQ ID NO: 7) comprises a frameshift mutation resulting in a pre-mature stop codon, wherein the encoded variant GIIα protein (abbreviated, variant “GIIα^{Stop}”) comprises a truncated amino acid sequence set forth in SEQ ID NO: 8. This frameshift mutation is present in T. reesei strain NG14, the last common ancestor of strains Rut-C30 and RL-P37.

As used herein, a Trichoderma strain named “Phy” is a parental Trichoderma strain comprising an introduced phytase expression cassette and the gls2α^{Stop} allele. The endogenous cellulase genes (i.e., cbh1, cbh2, egl1, egl2) were previously deleted from the genome.

As used herein, a modified Trichoderma strain named “Phy-gls2a^{R}” was derived from the Phy parent strain comprising the phytase expression cassette, wherein the Phy-gLs2a^{R} strain comprises a restored glsa2 (gls2a^{R}) allele.

As used herein, a Trichoderma strain named “GA” is a parental Trichoderma strain comprising an introduced glucoamylase (GA) expression cassette and the gls2α^{Stop} allele. The endogenous cellulase genes (i.e., cbh1, cbh2, egl1, egl2) were previously deleted from the genome.

As used herein, a modified Trichoderma strain named “GA-gls2a^{R}” was derived from the GA parent strain comprising the GA expression cassette, wherein the GA-gls2a^{R} strain comprises a restored glsa2 (gls2a^{R}) allele.

As used herein, a “restored glsa2 allele” (abbreviated, “gls2a^{R}”) refers to insertion of a single nucleotide base that restores the wild type reading frame and removes of the pre-mature stop codon present in the gls2α^{Stop} allele (SEQ ID NO: 7), wherein the restored gls2a^{R} allele encodes the native GIIα protein of SEQ ID NO: 6. For example, in certain embodiments, parental T. reesei cells/strains comprise a variant gls2a allele (gls2a^{Stop}) encoding the truncated GIIα¹ protein, wherein genetically modified T. reesei strains derived or obtained therefrom comprises a restored gls2a^{R} allele encoding the native GIIα protein.

As used herein, a modified Trichoderma strain named “Phy-Δmds2” was derived from the Phy parent strain comprising the phytase expression cassette and the gls2α^{Stop} allele, wherein the Phy-Δmds2 strain further comprises a deletion (Δ) of the mds2 (Δmds2) gene.

As used herein, the Trichoderma strain named “Lip” is a parental Trichoderma strain comprising an introduced lipase expression cassette and the gls2α^{Stop} allele. The endogenous cellulase genes (i.e., cbh1, cbh2, egl1, egl2) were previously deleted from the genome.

As used herein, a modified Trichoderma strain named “Lip-Δmds1” was derived from the Lip parent strain comprising the lipase expression cassette and the gls2α^{Stop} allele, wherein the Lip-Δmds1 strain further comprises a deletion (A) of the mds1 (Δmds1) gene.

As used herein, the phrase “Endo T gene product” refers to a secreted protein (i.e., an Endo-N-acetyl-β-D-glucosaminidase; abbreviated “ENGase”), which is a member of the glycoside hydrolase (GH) family 18 of deglycosylating enzymes. For example, Stals et al. 2012 (incorporated herein by reference in its entirety) describe the expression, purification and structural analysis of the ENGase (Endo T) from the mesophilic fungus Hypocrea jecorina (anamorph Trichoderma reesei), wherein glycosylation analysis of the cellulases secreted by the H. jecorina “Endo T knock-out strain” demonstrated in vivo function of the ENGase. Glycan cleavage catalyzed by the ENGase occurs between the two (2) basal GlcNAc residues, leaving the protein with only a single GlcNAc residue.

As used herein, the phrase “Endo T deleted” allele (abbreviated “ETD” allele) particularly refers to filamentous fungal strains having genetic modifications of the Endo T allele which render the strain deficient in the production of the native (functional) ENGase. More particularly, the Endo T allele of the recombinant (modified) T. reesei cells/strains described herein and set forth below in the Examples has been genetically modified to render the strains completely (100%) deficient in the production of an Endo T protein or an Endo T homolog. In particular, by reference to the wild-type T. reesei (H. jecorina) Endo T allele (SEQ ID NO: 36), one of skilled in the art can identify related EndoT genes in other filamentous fungal strains of interest. For example, fungal strains comprising the ETD allele are unable to cleave between the two (2) basal GlcNAc residues and thus retain larger N-linked glycan chains on secreted proteins (Stals et al., 2012).

As used herein, the Trichoderma strain named “Cel” is a parental Trichoderma strain comprising an introduced cellulase expression cassette and the gls2α^{Stop} allele. The strain was constructed from one in which the endogenous cellulase genes (i.e., cbh1, cbh2, egl1, egl2) were previously deleted from the genome. Genes (cbh1, cbh2, egl1, egl2) encoding the native cellulases were re-introduced as a single integrated expression cassette.

As used herein, a modified Trichoderma strain named “Cel-gls2a” was derived from the Cel parent strain comprising the cellulase expression cassette, wherein the Cel-gls2a^{R} strain comprises a restored glsa2 (gls2a^{R}) allele.

As used herein, the term “Endo H”, and related phrases, such as “Endo H treatment” and the like particularly refer to an enzyme treatment which de-glycosylates (removes) mannose (Man) from glycoproteins. In particular, enzymes having Endo H activity (i.e., endoglycosidase activity), such as the “Endoglycosidase H” (EndoH, New England BioLabs), are recombinant glycosidases which cleave within the chitobiose core of high mannose, and some hybrid oligosaccharides from N-linked glycoproteins.

As used herein, the terms “wild-type” and “native” are used interchangeably and refer to genes, proteins, fungal cells, or strains as found in nature.

As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions, or other functional alteration of a cell's genetic material. For example, recombinant cells may express genes or other nucleic acid molecules that are not found in identical or homologous form within a native (wild-type) cell, or may provide an altered expression pattern of endogenous genes, such as being over-expressed, under-expressed, minimally expressed, or not expressed at all.

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “gene” means the segment of DNA involved in producing a polypeptide (protein) chain, that may or may not include regions preceding and following the coding region (e.g., 5′ untranslated (5′ UTR) or “leader” sequences, 3′ UTR or “trailer” sequences, promoter sequences, terminator sequences and the like) as well as intervening sequences (introns) between individual coding segments (exons). For example, a gene (DNA) sequence of interest (GOI) may encode a structural protein, commercially important industrial proteins or peptides, such as enzymes (e.g., proteases, mannanases, xylanases, amylases, glucoamylases, cellulases, oxidases, phytases, lipases) and the like. The gene of interest may be a naturally occurring gene, a mutated (modified) gene or a synthetic gene.

As used herein, a “functional protein” is a protein that possesses an activity or function, such as an enzymatic activity, a binding function/activity (e.g., DNA binding), a surface-active property, and the like, and which has not been mutagenized, truncated, or otherwise modified to abolish or reduce that function or activity. Functional polypeptides can be thermostable or thermolabile, as specified.

As used herein, a “functional gene” is a gene capable of being used by cellular components to produce an active gene product, typically a protein. In contrast, a “non-functional gene” cannot be used by cellular components to produce an active gene product (i.e., a functional protein), or has a reduced ability to be used by cellular components to produce an active gene product (i.e., a functional protein).

As used herein, the term “promoter” refers to a nucleic acid sequence that functions to direct transcription of a downstream gene coding sequence (CDS; or open reading frame (ORF)). The promoter will generally be appropriate to the host cell (e.g., a fungal cell) in which the target gene is being expressed.

The promoter together with other transcriptional and translational regulatory nucleic acid sequences (also termed “control sequences”) is necessary to express a given gene. In general, the transcriptional and translational regulatory sequences include, but are not limited to, promoter and terminator sequences including a core promoter and enhancer or activator or repressor sequences, transcriptional and translational start and stop sequences. In certain embodiments, the promoter is an inducible promoter, a constitutive promoter, a tunable promoter, a synthetic promoter, a tandem promoter, and combinations thereof. In certain embodiments, the inducible promoter is an inducible cellulase gene promoter.

As used herein, the term “promoter activity” is the ability of a nucleic acid to direct transcription of a downstream (3′) polynucleotide in a host cell. To test promoter activity, the (promoter) nucleic acid may be operably linked to a downstream polynucleotide to produce a recombinant nucleic acid. The recombinant nucleic acid may be introduced into a cell, and transcription of the polynucleotide may be evaluated. In certain cases, the polynucleotide may encode a protein, and transcription of the polynucleotide can be evaluated by assessing production of the protein in the cell.

As used herein, the term “operably linked” refers to a functional linkage between two or more nucleic acid sequences. Thus, a nucleic acid sequence is operably linked when it is placed into a functional relationship with another nucleic acid sequence. For example, a promoter sequence or a terminator sequence is operably linked to a gene coding sequence (CDS) if it affects the transcription of the CDS; a ribosome binding site is operably linked to a coding sequence if it is positioned so as to facilitate translation; a nucleic acid sequence encoding a secretory leader (i.e., a signal peptide) is operably linked to a nucleic acid sequence (e.g., an ORF) encoding a polypeptide if it is expressed as a pre-protein that participates in the secretion of the polypeptide. Generally, “operably linked” means that the DNA (nucleic acid) sequences being linked are contiguous, and, in the case of a secretory leader, contiguous and in reading phase. However, enhancers do not have to be contiguous. Linking two or more nucleic acid sequences (i.e., operably linking) is accomplished using any of the methods to one of skill in the art.

As used herein, the phrases “lignocellulosic degrading enzymes”, “cellulase enzymes”, and “cellulases” are used interchangeably, and include glycoside hydrolase (GH) enzymes such as cellobiohydrolases, xylanases, endoglucanases, and β-glucosidases, that hydrolyze glycosidic bonds of cellulose (hemi-cellulose) to produce sugars (e.g., glucose, xylose, arabinose, etc.).

As used herein, “endoglucanase” proteins may be abbreviated as “EG”, “cellobiohydrolase” proteins may be abbreviated “CBH”, “β-glucosidase” proteins may be abbreviated “BG” and “xylanase” proteins may be abbreviated “XYL”. Thus, as used herein, a gene (or ORF) encoding a EG protein may be abbreviated “eg”, a gene (or ORF) encoding a CBH protein may be abbreviated “cbh”, a gene (or ORF) encoding a BG protein may be abbreviated “bg”, and a gene (or ORF) encoding a XYL protein may be abbreviated “xyl”. In certain embodiments, cellobiohydrolases include enzymes classified under Enzyme Commission No. (EC 3.2.1.91), endoglucanases include enzymes classified under EC 3.2.1.4, endo-β-1,4-xylanases include enzymes classified under EC 3.2.1.8, β-xylosidases include enzymes classified under EC 3.2.1.37. and β-glucosidases include enzymes classified under EC 3.2.1.21.

As used herein, a “cellulase gene promoter” includes, but is not limited to, a cellobiohydrolase (cbh) gene promoter sequence, an endoglucanase (eg) gene promoter sequence, a β-glucosidase (bg) gene promoter sequence, a xylanase (xyl) gene promoter sequence, and the like.

As used herein, the terms “modification” and “genetic modification” are used interchangeably and include: (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) the down-regulation and/or up-regulation of a gene, (f) specific mutagenesis and/or (g) random mutagenesis of any one or more the genes/DNA sequences disclosed herein.

As used herein, the phrases “modified filamentous fungal cell(s)”, “mutant filamentous fungal cell(s)”, “recombinant fungal cell(s)”, “modified filamentous fungal strain(s)”, and the like may be used interchangeably and refer to filamentous fungal cells that are derived (i.e., obtained) from a parental or control filamentous fungal cell belonging to the Pezizomycotina subphylum. For example, a “modified” filamentous fungal cell may be derived (obtained) from a parental or control filamentous fungal cell, wherein the modified cell comprises at least one genetic modification which is not found in the parental or control cell.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a functional protein). Exemplary methods of gene disruptions include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., the functional protein).

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of part or all of the coding sequence, and optionally adjacent enhancer elements, including but not limited to, for example, promoter and/or terminator sequences.

As used herein, a “heterologous gene” refers to polynucleotide (DNA) sequences having at least a portion of the sequence which is not native or existing in a native form to the cell in which it is introduced and/or expressed.

As used herein, a “heterologous nucleic acid construct” or “heterologous DNA sequence” has a portion of the sequence which is not native or existing in a native form to the cell in which it is expressed.

As used herein, a “heterologous protein” is encoded by a heterologous gene, a heterologous nucleic acid (polynucleotide) sequence, a heterologous DNA sequence, and the like.

Thus, in certain embodiments, a heterologous gene, a heterologous nucleic acid construct, a heterologous DNA sequence, etc. encoding a protein of interest (POI) is introduced (e.g., transformed) into a filamentous fungal cell (strain). For example, a heterologous gene construct encoding a POI may be introduced into the filamentous fungal cell (strain) before, during, or after performing other genetic modification described herein.

Heterologous, with respect to a control sequence refers to a control sequence (e.g., promoters, enhancers, terminators) that does not function in nature to regulate the same gene the expression of which it is currently regulating. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

As used herein, the term gene “coding sequence” (abbreviated, “CDS”) refers to a polynucleotide sequence, which directly specifies the amino acid sequence of its (encoded) protein product. The boundaries of the CDS arc generally determined by an open reading frame (ORF), which usually begins with a start codon (ATG). The coding sequence typically includes DNA, cDNA, and recombinant nucleotide sequences. For example, an ORF generally refers to polynucleotide sequence (whether naturally occurring, non-naturally occurring, or synthetic) comprising an uninterrupted reading frame consisting of (i) an initiation codon, (ii) a series of codons representing amino acids of the encoded protein product, and (iii) a termination codon, the ORF being read (or translated) in the 5′ to 3′ direction.

As used herein, the term “DNA construct” or “expression construct” refers to a nucleic acid sequence, which comprises at least two DNA polynucleotide fragments. A DNA or expression construct can be used to introduce nucleic acid sequences into a fungal host cell. The DNA may be generated in vitro (e.g., by PCR) or any other suitable techniques. In some embodiments, the DNA construct comprises a sequence of interest (e.g., encoding a protein of interest). In certain embodiments, a polynucleotide sequence of interest is operably linked to a promoter and/or a terminator. In some embodiments, the DNA construct further comprises at least one selectable marker. In further embodiments, the DNA construct comprises sequences homologous to the host cell chromosome. In other embodiments, the DNA construct comprises non-homologous sequences to the host cell chromosome.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the filamentous fungal chromosome. These sequences direct where in the filamentous fungal chromosome the new construct gets integrated and what part, if any, of the chromosome will be replaced by the incoming sequence.

As used herein, the term “down-regulation” of gene expression includes any methods that result in lower (down-regulated) expression of a functional gene product.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes, and the like. Expression vectors may include regulatory sequences such as promoters, signal sequences, coding sequences and transcription terminators.

An “expression vector” as used herein means a DNA construct comprising a coding sequence that is operably linked to suitable control sequences capable of effecting expression of a protein in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the term “secretory signal sequence” denotes a DNA sequence that encodes a polypeptide (i.e., a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

As used herein, the term “isolated” or “purified” refers to a filamentous fungal cell, a nucleic acid or a polypeptide that is removed from at least one component with which it is naturally associated.

As used herein, the term “protein of interest” (POI) refers to a polypeptide that is desired to be expressed in a filamentous fungal cell. Such a protein can be an enzyme, a substrate-binding protein, a surface-active protein, a structural protein, and the like, and can be expressed at high levels, and can be for the purpose of commercialization. For example, as generally set forth below, a POI includes, but is not limited to, phytases, glucoamylases, cellulases, hemicellulases, xylanases, peroxidases, proteases, lipases, phospholipases, esterases, cutinases, polyesterases, pectinases, keratinases, reductases, oxidases, phenol oxidases, lipoxygenases, ligninases, pullulanases, tannases, pentosanases, mannanases, α-glucanases, β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, isomerases, and the like.

A protein of interest (POI) can be encoded by an “endogenous” gene. For example, in certain embodiments, a POI is encoded by a gene endogenous to the filamentous fungal cell (strain), such as the aforementioned wild-type genes encoding the native suite of cellulases (e.g., cellobiohydrolases, xylanases, endoglucanases and β-glucosidases).

As used herein, the term “increased productivity” and variations thereof mean an increase of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the production of a protein of interest by a modified (mutant) filamentous fungal cell relative to a parental (control) filamentous fungal cell, when cultivated under the same conditions (e.g., medium composition, temperature. pH, cell density, dissolved oxygen, time, etc.).

As used herein, the term “increased amount” when used in phrases such as a recombinant cell “produces an ‘increased amount’ of a protein of interest”, and variations thereof mean an increase of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (e.g., greater than 20%) in the amount of a protein of interest produced by a modified (mutant) filamentous fungal cell relative to a parental (control) filamentous fungal cell, when cultivated under the same conditions.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention (e.g., disulfide bond formation, glycosylation, lipidation, acetylation, phosphorylation, or any other manipulation or modification, such as conjugation with a labeling component). Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the phrase “substantially free of an activity,” or similar phrases, means that a specified activity is either undetectable in an admixture or present in an amount that would not interfere with the intended purpose of the admixture.

As used herein, the term “derivative polypeptide” refers to a protein which is derived or derivable from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative can be achieved by modifying a DNA sequence which encodes for the native protein, transformation of that DNA sequence into a suitable host, and expression of the modified DNA sequence to form the derivative protein.

Related (and derivative) proteins include “variant proteins.” Variant proteins differ from a reference/parental protein (e.g., a wild-type protein) by substitutions, deletions, and/or insertions at a small number of amino acid residues. The number of differing amino acid residues between the variant and parental protein can be one or more, for example, 1, 2, 3, 4, 5, 6, 7, 8, 9, 10, 15, 20, 30, 40, 50, or more amino acid residues. Variant proteins can share at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a reference protein. A variant protein can also differ from a reference protein in selected motifs, domains, epitopes, conserved regions, and the like.

As used herein, the term “homologous” protein refers to a protein that has similar activity, function and/or structure to a reference protein. It is not intended that homologs necessarily be evolutionarily related. Thus, it is intended that the term encompass the same, similar, or corresponding protein(s) (i.e., in terms of structure and function) obtained from different organisms. In some embodiments, it is desirable to identify a homolog that has a quaternary, tertiary and/or primary structure similar to the reference protein. For example, one or more gls2a, mds1 and/or mds2 genes from filamentous fungal strains such as A. niger, A. oryzae, M. thermophila (T. thermophila), and the like, encoding protein homologues comprising substantial amino acid sequence identity to the full-length T. reesei Gls2a, Mds1 and/or Mds2 proteins of the disclosure, are readily identified in publicly available genome databases using methods further outlined below in Example 4.

The degree of homology between sequences can be determined using any suitable method known in the art (see, e.g., Smith and Waterman, 1981; Needleman and Wunsch. 1970; Pearson and Lipman, 1988; programs such as GAP, BESTFIT, FASTA, and TFASTA in the Wisconsin Genetics Software Package (Genetics Computer Group, Madison, WI); and Devereux et al., 1984). For purposes of the present invention, the degree of identity between two amino acid sequences is determined using the Needleman-Wunsch algorithm (Needleman and Wunsch, 1970) as implemented in the Needle program of the EMBOSS package (Rice et al., 2000), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

As used herein, the phrases “substantially similar” and “substantially identical”, in the context of at least two nucleic acids or polypeptides, typically means that a polynucleotide or polypeptide comprises a sequence that has at least about 40% identity, at least about 50% identity, at least about 60% identity, at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity can be determined using known programs such as BLAST, ALIGN, and CLUSTAL using standard parameters.

As used herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, ultrafiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “protein preparation” is any material, typically a solution, generally aqueous, comprising one or more proteins.

The terms “recovery”, “recovered” and “recovering” as used herein refer to treatment or stabilization of broth, or at least partial separation of a protein from one or more soluble and/or insoluble components of a microbial broth and/or at least partial separation from one or more solvents in the broth (e.g., water or ethanol). A recovered protein is often of higher purity than prior to the recovery process.

However, in some embodiments, a recovered protein may be of the same or lower purity than prior to the recovery process.

As used herein, the terms “broth”, “cultivation broth”, “fermentation broth” and/or “whole fermentation broth” may be used interchangeably, and refer to a preparation produced by cellular fermentation that undergoes no processing steps after the fermentation is complete. For example, whole fermentation broths are typically produced when filamentous fungal cells are grown to saturation, incubated under carbon-limiting conditions to allow protein synthesis (e.g., expression and secretion of the proteins into cell culture medium). Typically, the whole fermentation broth is unfractionated and comprises spent cell culture medium, metabolites, extracellular polypeptides, and microbial cells.

As used herein, the phrase “treated broth” refers to a broth that has been conditioned by making changes to the chemical composition and/or physical properties of the broth. Broth “conditioning” may include one or more treatments or steps, such as filtration, diafiltration, cell lysis, pH modification, heating, cooling, addition of chemicals (e.g., calcium, salt(s), flocculant(s), reducing agent(s), enzyme activator(s), enzyme inhibitor(s), and/or surfactant(s)), mixing, and/or timed hold (e.g., 0.5 to 200 hours) of the broth without further treatment.

As used herein, “aerobic fermentation” refers to growth in the presence of oxygen.

As used herein, the term “cell mass” refers to the cell component (including intact and lysed cells) present in a liquid/submerged culture. Cell mass can be expressed in dry or wet weight.

As used herein, when the expression/production of a protein of interest (POI) in an “unmodified” (parental or control) cell is being compared to the expression/production of the same POI in a “modified” (recombinant) cell, it will be understood that the “unmodified” (control) and “modified (recombinant) cells are grown/cultivated/fermented under the same conditions (e.g., the same conditions such as media, temperature, pH and the like). Thus, a POI of the disclosure may be produced inside the host cell, or secreted (or transported) into the culture medium.

It will be understood that the methods of the present disclosure are not limited to a particular order for obtaining the modified (mutant) filamentous fungal cell (strain). The modification of a gene may be introduced into the parent strain at any step in the construction of the strain for the production of an endogenous and/or heterologous protein of interest (POI).

### II. Glycoproteins

As briefly set forth in the Background, filamentous fungi (e.g., Trichoderma sp., Aspergillus sp., Myceliophthora sp., and the like) are useful in the production of proteins of interest, and as such are often exploited for industrial production of recombinant proteins. In certain embodiments, recombinant (modified) fungal strains are particularly suitable for use in the production of glycoproteins. As generally understood by one skilled in the art, glycosylated proteins (glycoproteins) produced in filamentous fungal strains can be N-linked, e.g., glycan linked to nitrogen (N) of amide group of L-asparagine, and/or can be O-linked, e.g., glycan linked to the hydroxyl groups (O) of L-serine or L-threonine. In most eukaryotic organisms, N-linked glycans are first synthesized as lipid-linked tetradecasaccharides (14-sugar units comprising a “Glc3Man9GlcNAc2” precursor) that is transferred within the lumen of the ER onto suitable L-asparagine (N-linked) residues of a nascent polypeptide chain (Kornfeld and Kornfeld, 1985). After transfer to the protein, the terminal α-1,2-linked glucose (Glc) is rapidly removed by α-glucosidase I (GI), while the two innermost α-1,3-linked glucose (Glc) residues are cleaved off via the action of α-glucosidase II (GII).

The α-glucosidase II protein (abbreviated, “GII” protein) is generally accepted to be an asymmetric non-globular heterodimer comprising a catalytic alpha subunit (GIIα) and a beta subunit (GIIβ). Geysens et al. (2005) have described the isolation and characterization of the gene encoding the α-glucosidase II alpha subunit (GIIα) from the Trichoderma strain Rut-C30, wherein the gls2a gene encoding the GIIα subunit in the hypercellulolytic strain Rut-C30 contains a frameshift mutation resulting in a truncated GIIα protein, wherein peculiar monoglucosylated N-glycan patterns on proteins produced by the strain were observed. In particular, Geysens et al. (2005) concluded that the truncated GIIα protein can still hydrolyze the first α-1,3-linked glucose (Glc) residue, but not the innermost α-1,3-linked glucose (Glc2) residues from the Glc2Man9GlcNAc2 N-glycan ER structure. As described in Geysens et al. (2005), transformation of the Rut-C30 strain with a plasmid encoding the wild type T. reesei GIIα subunit significantly changed the glycosylation profile, decreasing the amount of monoglucosylated structures and increasing the overall heterogeneity and amount of high-mannose N-glycans. Full conversion to high-mannose carbohydrates (e.g., Man9GlcNAc2) was not obtained, which was postulated to be due to competition between the endogenous mutant (truncated) GIIα^{Stop} subunit and the introduced wild-type (native) GIIα subunit. More recent crystallographic characterization of a fungal GIIα subunit has been described by Satoh et al. (2016), which suggests that the T. reesei gls2a frameshift mutant (gls2a^{STOP}) would have diminished contact with the GIIβ subunit.

As set forth herein and further described below in the Examples section, Applicant has evaluated the specific roles of the gls2a frameshift mutation (gls2a^{STOP} in the glycosylation profiles of both native (endogenous) enzymes and heterologously expressed enzymes. More particularly, as described in the Examples, reporter strains derived from a common ancestor to a T. reesei Rut-C30 strain were generated, expressing (native) cellulases or a heterologous phytase reporter glycoprotein. For example, rather than supplying an ectopic, full-length copy of gls2a allele described by Geysens et al. (2005), Applicant has repaired (i.e., restored) the original (endogenous) frameshift mutation in each reporter strain described, thereby restoring the correct reading frame of the of gls2a allele (i.e., allele gls2a^{R}).

In particular, as presented and described in Example 3, the N-glycan pattern of these reporter strains become remarkably homogeneous, consisting primarily of Man5GlcNAc2 for both the cellulase reporter strain and the phytase reporter strain, whereas no high mannose structures were detected. This result is strikingly different from that previously found (Geysens et al 2005) where instead of replacing the gls2a^{STOP} with the revertant allele, a strain containing both the gls2a^{STOP} and a wild-type version was studied. When both alleles were present, the N-linked glycans contained substantial amounts of monoglucosylated and high mannose chains.

As presented and described in Example 1, Applicant has found that replacing the gls2a^{STOP} with the revertant allele in the glucoamylase reporter strain resulted in a significant increase in glucoamylase production. This result suggests that processing of N-glycans in the secretory pathway can in some cases be rate limiting for protein production.

Likewise, the post-production release of mannose (Man) from glycoproteins, oligosaccharides and the like is a potential source of reducing sugars that can chemically react with surface lysine residues, a phenomenon known as glycation, protein glycation, enzyme glycation and the like. For example, protein glycation can have negative impacts on the storage stability of industrially produced enzymes as set forth in Sutthirak et al. (2005), as well as those described herein. However, the precise source and mechanism by which the free mannose is produced is generally unknown. Thus, characterization of the mechanism(s) of mannose production will further aid in the mitigation of mannose release, aid in the mitigation of protein glycation events, mitigate protein product activity losses, enhance protein product storage stability, and the like.

Based on the foregoing, it is highly desirable in the art to understand the sources of free mannose and related mechanisms of production of the free mannose. In particular, the secretion of proteins with overall reduced glycan content, observed for gls2a^{R} strains (Examples 1 and 3), was used to demonstrate the role of glycoproteins in the generation of free sugars during post-fermentation recovery and processing (Example 2). In addition, Applicant identified two 1,2-α-D-mannosidases (i.e., Mds1 and Mds2) in the media during T. reesei fermentations. For example, the alpha-mannosidase-1 (Mds1) has been described in Maras et al. (2000). Although this enzyme exhibited mannosidase activity when heterologously expressed in yeast (e.g., Pichia), no mannosidase activity was detected in culture supernatants of the T. reesei host strain from which the gene was cloned.

Thus, to determine the potential role of two 1,2-α-D-mannosidases (Mds1 and/or Mds2) in release of mannose in soluble extracts, genes encoding these proteins were deleted as described below in the Examples. The substrates for Mds1 and Mds2 can be secreted glycoproteins, cell wall oligosaccharides, and/or other materials. In particular, as described in the Examples, deletions of each mannosidase gene caused a reduction of free mannose levels, demonstrating their critical role in mannose release. More specifically, deletion of the mannosidase genes resulted in a significant reduction of mannose release and a concomitant reduction in the amount of unwanted protein glycation in the media during fermentations.

Thus, as described herein, certain embodiments of the disclosure are related to, inter alia, recombinant filamentous fungal cells (strains) producing proteins of interest, methods and compositions for the design and construction of recombinant (modified) fungal cells producing proteins of interest, methods and compositions for the expression/production of endogenous proteins of interest in recombinant filamentous fungal cells, methods and compositions for the expression/production of heterologous proteins of interest in recombinant filamentous fungal cells, methods and compositions for producing proteins of interest in recombinant filamentous fungal, wherein the proteins produced and/or recovered therefrom have uniform and consistent N-linked glycosylation patterns, and/or consistent monoglucosylated structures and/or reduced (unwanted) protein of interest glycation events, and the like. Thus, certain other embodiments are related to proteins or glycoproteins produced (secreted) by recombinant fungal strains of the disclosure. In related embodiments, the strains, compositions and methods of the disclosure provide proteins/glycoproteins having improved or enhanced whole broth processing properties, proteins/glycoproteins having improved or enhanced downstream recovery and purification properties, improved or enhanced protein/glycoprotein product profiles such as stability, activity, shelf-life, etc., improved quality control (QC), reduced mannose (Man) release, and the like.

### III. Recombinant Nucleic Acids, Molecular Biology and Methods for Contructing Modified Filamentous Fungal Strains

As described above, certain embodiments are related to recombinant (modified) fungal strains derived from parental strains comprising native genes encoding a functional Mds1 protein and/or protein Mds2 protein and/or derived from parental strains comprising mutated genes encoding truncated GIIα proteins. In certain embodiments, modified strains comprise genetic modifications rendering the modified strains deficient in the production of one or more native genes encoding a functional Mds1 and/or Mds2 protein. In other embodiments, fungal strains comprising a variant gls2a gene (gls2a^{Stop}) encoding a truncated GIIα^{Stop} protein of the disclosure are genetically modified herewith to replace the variant gls2a^{Stop} with a restored copy of the wild-type gls2a gene (gls2a^{R}) encoding the native full length GIIα protein. As noted above, various methods and techniques are available for constructing, screening, identifying, selecting and the like, one or more modified strains of the disclosure.

Thus, in one or more embodiments of the disclosure, modified, mutant, or recombinant filamentous fungal cells/strains of the disclosure are deficient in the production of one or mannosidases set forth and described herein (i.e., compared to (vis-1-vis) one or more control/parental cells). In certain embodiments, modified filamentous fungal cells deficient in the production of one or more mannosidases produce no detectable activity of one or more (several) mannosidase enzymes compared to a parent (control) filamentous fungal cell/strain when cultivated under identical conditions, or, in the alternative, produces preferably at least 25% less, more preferably at least 50% less, even more preferably at least 75% less, and most preferably at least 95% less of one or more (several) mannosidase enzymes than the parent/control filamentous fungal cell when cultivated under identical conditions. The level of the one or more mannosidases produced by filamentous fungal cells of the disclosure may be determined using methods described herein and/or methods known in the art.

Thus, certain embodiments are related to modified fungal strains comprising one or more genetic modifications, one or more introduced nucleic acids (e.g., expression cassettes, targeting vectors, etc.) and the like. In any of these embodiments, the modified, mutant, parental and/or control strains may comprise additional genetic modifications described herein. Certain embodiments are therefore related to recombinant microbial strains, recombinant polynucleotides, plasmids, vectors, expression cassettes and the like. In certain embodiments, modified (recombinant) filamentous fungal strains described herein express one or more (heterologous or endogenous) proteins of interest.

Thus, in certain embodiments, one or more genetic elements, e.g., a promoter sequence, a gene coding sequence (CDS), a 5′-UTR sequence, a vector, a polynucleotide, and the like, may be genetically modified, as generally understood by one skilled in the art. In certain embodiments, genetic modifications include, but are not limited to, (a) the introduction, substitution, or removal of one or more nucleotides in a gene, or the introduction, substitution, or removal of one or more nucleotides in a regulatory element required for the transcription or translation of the gene, (b) a gene disruption, (c) a gene conversion, (d) a gene deletion, (e) the down-regulation of a gene, (f) the overexpression (OE) of a gene, (g) specific mutagenesis and/or (h) random mutagenesis of any one or more the genes disclosed herein.

As further described herein, one or more methods/techniques set forth above may be used to construct one or more genetically modified filamentous fungal strains of the disclosure. Such methods are particularly suitable for constructing modified filamentous fungal cells/strains deficient in production of one or more functional proteins. For instance, in certain one or more embodiments of the disclosure, modified filamentous fungal cells are genetically modified to render the cells deficient in the production of one or more mannosidases.

Thus, in certain embodiments, modified filamentous fungal cells of the disclosure are constructed by reducing or eliminating the expression of one or more genes set forth above, using methods well known in the art, for example, insertions, disruptions, replacements, or deletions. The portion of the gene to be modified or inactivated may be, for example, the coding region or a regulatory element required for expression of the coding region. An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator, and the like.

In certain other embodiments, modified filamentous fungal cells may be constructed by gene deletion to eliminate or reduce the expression of at least one of the aforementioned genes of the disclosure. Gene deletion techniques enable the partial or complete removal of the gene(s), thereby eliminating their expression, or expressing a non-functional (or reduced activity) protein product. In such methods, the deletion of the gene(s) may be accomplished by homologous recombination using a plasmid that has been constructed to contiguously contain the 5′ and 3′ regions flanking the gene. The contiguous 5′ and 3′ regions may be introduced into a fungal cell, for example, on a temperature-sensitive plasmid, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is affected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers. Thus, a person of skill in the art, e.g., by reference to one or more genes and/or protein sequences of the disclosure, may readily identify nucleotide regions in the gene's coding sequence and/or the gene's non-coding sequence suitable for complete or partial deletion.

In other embodiments, modified filamentous fungal cells of the disclosure are constructed by introducing, substituting, or removing one or more nucleotides in the gene, or a regulatory element required for the transcription or translation thereof. For example, nucleotides may be inserted or removed so as to result in the introduction of a stop codon, the removal of the start codon, or a frame shift of the open reading frame. Such modifications may be accomplished by site-directed mutagenesis or PCR generated mutagenesis in accordance with methods known in the art. Thus, in certain embodiments, one or more genes of the disclosure may be inactivated by complete or partial deletion.

In another embodiment, modified filamentous fungal cells may be constructed by the process of gene conversion, wherein a nucleic acid sequence corresponding to the gene is mutagenized in vitro to produce a defective nucleic acid sequence, which is then transformed into a parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is affected by selection for the marker under conditions not permitting plasmid replication. Selection for a second recombination event leading to gene replacement is affected by examination of colonies for loss of the selectable marker and acquisition of the mutated gene. Alternatively, the defective nucleic acid sequence may contain an insertion, substitution, or deletion of one or more nucleotides of the gene, as described below.

In certain embodiments, modified filamentous fungal cells may be constructed via CRISPR-Cas9 editing. For example, a gene of interest can be modified, disrupted, deleted, or down-regulated by means of nucleic acid guided endonucleases, that find their target DNA by binding either a guide RNA (e.g., Cas9 and Cpf1) or a guide DNA (e.g., NgAgo), which recruits the endonuclease to the target sequence on the DNA, wherein the endonuclease can generate a single or double stranded break in the DNA. This targeted DNA break becomes a substrate for DNA repair and can recombine with a provided editing template to disrupt or delete or modify the gene. For example, the gene encoding the nucleic acid guided endonuclease (for this purpose Cas9 from S. pyogenes) or a codon optimized gene encoding the Cas9 nuclease is operably linked to a promoter active in the fungal cell and a terminator active in a fungal cell, thereby creating a fungal Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (NGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a fungal cell expression cassette for the gRNA is created by operably linking the DNA encoding the gRNA to a promoter active in fungal cell and a terminator active in fungal cell. In other embodiments, purified Cas9 and gRNA may be commercially obtained, assembled in vitro, and introduced alone or with incoming DNA repair templates.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of the targeted gene can be fused to about 500 bp 3′ of the targeted gene to generate an editing template, which template is used by the fungal host's machinery to repair the DNA break generated by the RNA-guided endonuclease (RGEN). Even shorter stretches of nucleotides in the form of double or single stranded DNA can be used as an editing template.

The Cas9 expression cassette, the gRNA expression cassette or in vitro formed Cas9-gRNA complex (RNP) and the editing template can be co-delivered to filamentous fungal cells using many different methods (e.g., PEG mediated protoplast transformation, protoplast fusion, electroporation, biolistics). The transformed cells are screened by PCR amplifying the target gene with a forward and reverse primer. These primers can amplify the wild-type locus or the modified locus that has been edited by the RGEN.

For example, in certain embodiments, a parental T. reesei strain comprising a variant gls2a allele (gls2a^{Stop}) encoding a truncated GIIα^{Stop} protein is genetically modified herewith via such CRISPR-Cas9 editing systems. In particular, as set forth in the Examples, parental T. reesei strains (RLP37) comprising the variant gls2a^{Stop} allele were modified herein via CRISPR-Cas9 editing, wherein the modified strains obtained therefrom have a restored gls2a^{R} allele encoding the native GIIα protein. Those of skill in the art are well aware of suitable methods for introducing polynucleotides into filamentous fungal cells (e.g., Aspergillus sp., Trichoderma sp., etc.), wherein standard techniques for transformation of filamentous fungi and culturing the fungi (which are well known to one skilled in the art) are used to transform a fungal host cell of the disclosure. Thus, the introduction of a DNA construct or vector into a fungal host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation, protoplast fusion and the like. General transformation techniques are known in the art (see, e.g., Ausubel et al., 1987, Sambrook et al., 2001). The expression of heterologous proteins in Trichoderma has been described, for example, in U.S. Pat. Nos. 6,022,725; 6,268,328. Reference is also made to Cao et al. (2000), for transformation of Aspergillus strains.

In other embodiments, modified filamentous fungal cells are constructed by established anti-sense techniques, e.g., using a nucleotide sequence complementary to a nucleic acid sequence of interest. For example, expression of a functional gene by a filamentous fungal cell may be reduced (down-regulated), or eliminated, by introducing a nucleotide sequence complementary to the gene's nucleic acid sequence, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to, RNA interference (RNAi), small interfering RNA (siRNA), microRNA (miRNA), antisense oligonucleotides, and the like, all of which are well known to the skilled artisan.

In other embodiments, technologies can be used to identify any specific point mutation in an organism essentially using classical mutagenesis and then exhaustive screening. One such technique is called FIND-IT (Knudsen et al., 2022) in which genetically variable populations (from either natural variation or induced mutations) are pooled together and then systematically and repeatedly screened using highly sensitive PCR approaches to identify individuals containing the desired mutation.

In certain embodiments, additional means of restoring the gls2a^{Stop} allele that do not insert the original deleted nucleotide, will likely give rise to phenotypic restoration, e.g., adding a base a few codons upstream or downstream leads to a GIIα protein that only differs from the wild type by a few amino acids and retains a wild type function.

In certain other embodiments, the recombinant nucleic acid (or polynucleotide expression cassette thereof or expression vector thereof) further comprises one or more selectable markers. Selectable markers for use in filamentous fungi include, but arc not limited to, als1, amdS, hphB, pyr2, pyr4, pyrG, sucA trpC, argB, a bleomycin resistance marker, a blasticidin resistance marker, a pyrithiamine resistance marker, a neomycin resistance marker, an adenine pathway gene, a thymidine kinase marker and the like. In a particular embodiment, the selectable marker is pyr2, which compositions and methods of use are generally set forth in PCT Publication No. WO2011/153449.

Generally, transformation of Trichoderma sp. cells uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) is mixed with the desired DNA. Generally, a high concentration of polyethylene glycol (PEG) is added to the uptake solution. Additives, such as dimethyl sulfoxide, heparin, spermidine, potassium chloride and the like, may also be added to the uptake solution to facilitate transformation. Similar procedures are available for other fungal host cells. See, e.g., U.S. Pat. Nos. 6,022,725 and 6,268,328, both of which are incorporated by reference.

In certain embodiments, mutant strains comprise a genetic modification which replaces (substitutes) a native promoter sequence of an endogenous gene encoding a native protein of the disclosure with a heterologous promoter sequence. For example, in certain embodiments, a mutant strain comprises a knocked-in heterologous promoter sequence which drives the expression of the endogenous gene encoding the native protein. In other embodiments, a mutant strain comprises a knocked-out (or mutated) native promoter sequence of an endogenous gene encoding the functional protein, thereby rendering the mutant strain deficient in the production of the native protein.

In other embodiments, mutant (or modified) strains comprise one or more introduced nucleic acids expressing or overexpressing one or more proteins of interest. For example, in certain embodiments, a mutant strain comprises an introduced polynucleotide (expression cassette) comprising a heterologous promoter (pro) sequence upstream (5′) and operably linked a downstream (3′) nucleic acid encoding a protein of interest. Heterologous promoter (pro) sequences suitable for driving the expression or overexpression of a protein include any promoter sequences known to one skilled in the art, wherein particularly preferred promoters include any promoter sequences capable of increasing the expression of the protein in the desired fungal cell. In related embodiments, the cassette may further comprise a downstream (3′) transcriptional terminator sequence operably linked to the gene CDS.

As used herein, promoter and/or terminator sequences are not meant to be limiting but are rather selected so as to be functional in the desired fungal cell/strain. For example, a promoter sequence can be any nucleotide sequence that shows transcriptional activity in the filamentous fungal cell, including mutant/variant promoters, truncated promoters, tandem promoters, hybrid promoters, synthetic promoters, inducible promoters, tuned promoters, conditional expression systems and combinations thereof. Often, suitable promoters can be obtained from genes encoding extracellular or intracellular polypeptides either native or heterologous (foreign) to the filamentous fungal cell. Examples of promoters suitable for driving the expression of one or more regulatory genes of the disclosure include, but are not limited, to a Trichoderma reesei cDNA1 promoter, an enol promoter, a pdc1 promoter, a pki1 promoter, a tef) promoter, a rp2 promoter, a cbh1 promoter, a cbh2 promoter, an egl1 promoter, an egl2 promoter and other T. reesei promoters described in Fitz et al. 2018 (incorporated herein by referenced in its entirety), the Aspergillus oryzae thiA promoter, amylase promoter, the A. nidulans gpdA promoter, the A. niger glaA promoter and the like.

In certain embodiments, the instant disclosure is directed to the expression/production of one or more proteins of interest which are endogenous to the filamentous fungal host cell. In other embodiments, the disclosure is directed to expressing/producing one or more proteins of interest which are heterologous to the filamentous fungal host cell.

In certain embodiments, a heterologous gene is cloned into an intermediate vector, before being transformed into a filamentous fungal (host) cells for expression. These intermediate vectors can be prokaryotic vectors, such as, e.g., plasmids, or shuttle vectors. The expression vector/construct typically contains a transcription unit or expression cassette that contains all the additional elements required for the expression of the heterologous sequence. For example, atypical expression cassette contains a 5′ promoter operably linked to the heterologous nucleic acid sequence encoding the POI and may further comprise sequence signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes. Although any fungal terminator is likely to be functional in the present invention, preferred terminators include: the terminator from Trichoderma cbh1 gene, the terminator from Aspergillus nidulans trpC gene, the Aspergillus awamori or Aspergillus niger glucoamylase genes and/or the Mucor miehei carboxyl protease gene.

The particular expression vector used to transport the genetic information into the cell is not particularly critical. Any of the conventional vectors used for expression in eukaryotic or prokaryotic cells may be used. Standard bacterial expression vectors include bacteriophages a and M13, as well as plasmids such as pBR322 based plasmids, pSKF, pET23D, and fusion expression systems such as MBP, GST, and LacZ, as well as yeast 2μ plasmids and centromeric yeast plasmids. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in the fungal host.

The methods of transformation of the present disclosure may result in the stable integration of all or part of the transformation vector into the genome of the filamentous fungus. However, transformation resulting in the maintenance of a self-replicating extra-chromosomal transformation vector is also contemplated. Any of the known procedures for introducing foreign (heterologous) nucleotide sequences into host cells may be used. These include the use of calcium phosphate transfection, polybrene, protoplast fusion, electroporation, biolistics, liposomes, microinjection, and any of the other known methods for introducing cloned genomic DNA, cDNA, synthetic DNA or other foreign genetic material into a host cell (see, e.g., Sambrook et al., supra). Also of use is the Agrobacterium-mediated transfection method such as the one described in U.S. Pat. No. 6,255,115.

After the expression vector(s) is/are introduced into the cells, the transformed cells are cultured under conditions favoring expression of genes under control of the specific gene promoter used, which promoter is selected from optimal activity under the appropriate fermentation conditions and the like, of the choice. Large batches of transformed cells can be cultured as described herein. Finally, product is recovered from the culture using standard techniques.

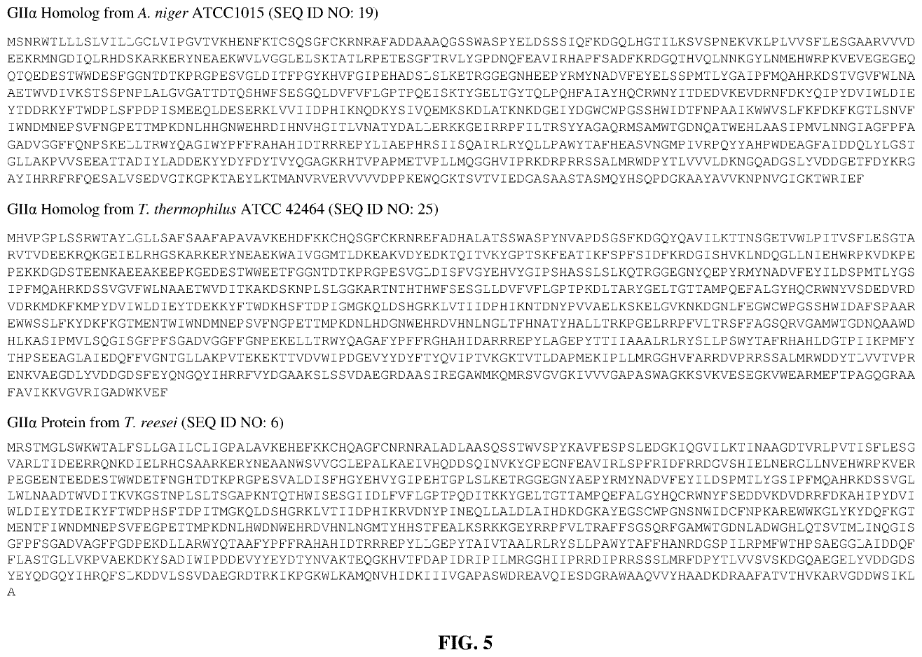

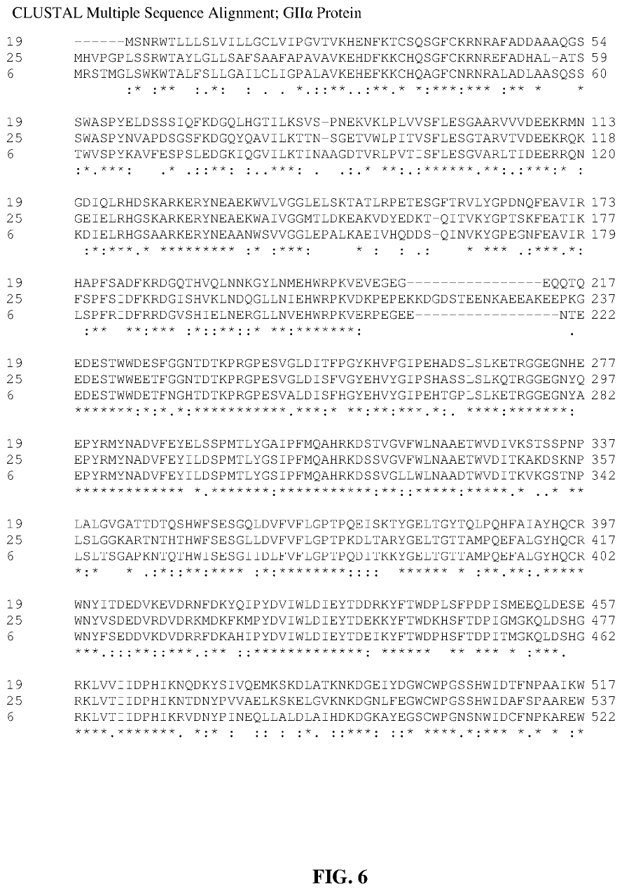

As further described and exemplified below (Example 4), Applicant screened suitable gene/protein sequence databases and identified several filamentous genes encoding GIIα, Mds1 and Mds2 protein homologues. For example, the T. reesei GIIα protein (SEQ ID NO: 6), an A. niger GIIα protein homologue (SEQ ID NO: 19; strain ATCC No. 1015) and a T. thermophilus GIIα protein homologue (SEQ ID NO: 25; strain ATCC No. 42464) are presented in FIG. 5.

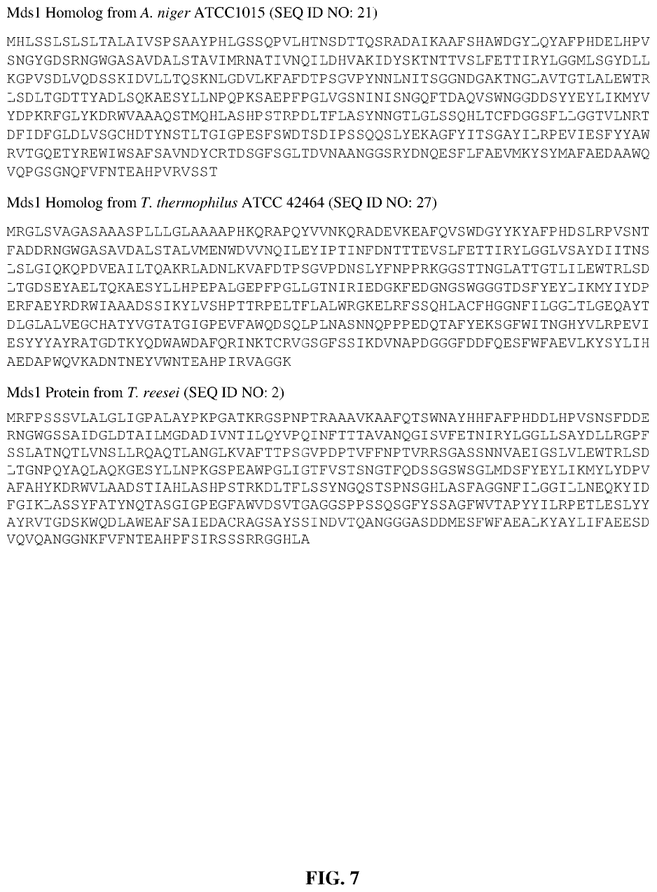

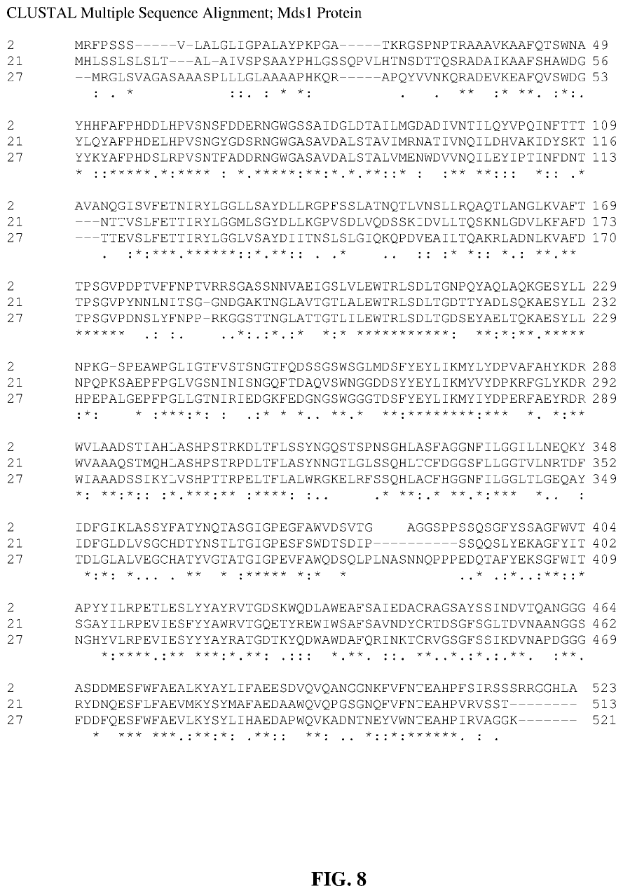

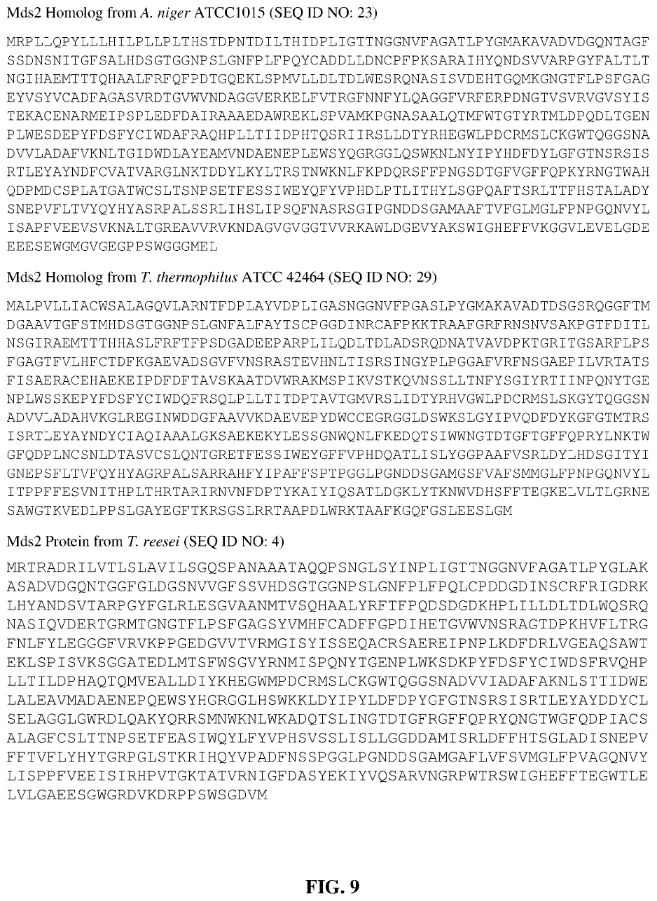

Likewise, the T. reesei Mds1 protein (SEQ ID NO: 2), an A. niger Mds1 protein homologue (SEQ ID NO: 21; strain ATCC No. 1015) and a T. thermophilus Mds1 protein homologue (SEQ ID NO: 27; strain ATCC No. 42464) are presented in FIG. 7, and the T. reesei Mds2 protein (SEQ ID NO: 4), an A. niger Mds2 protein homologue (SEQ ID NO: 23; strain ATCC No. 1015) and a T. thermophilus Mds2 protein homologue (SEQ ID NO: 29; strain ATCC No. 42464) are presented in FIG. 9.

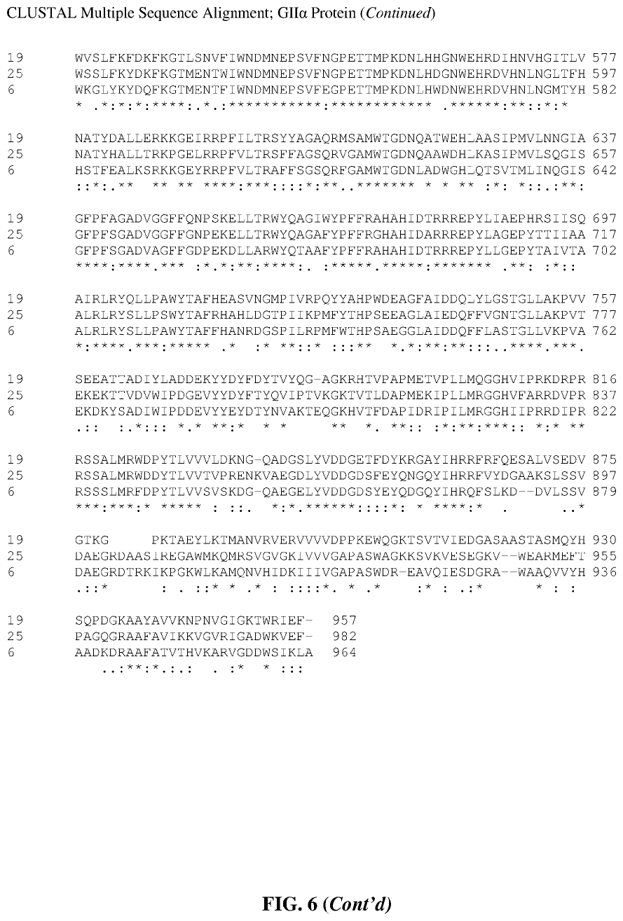

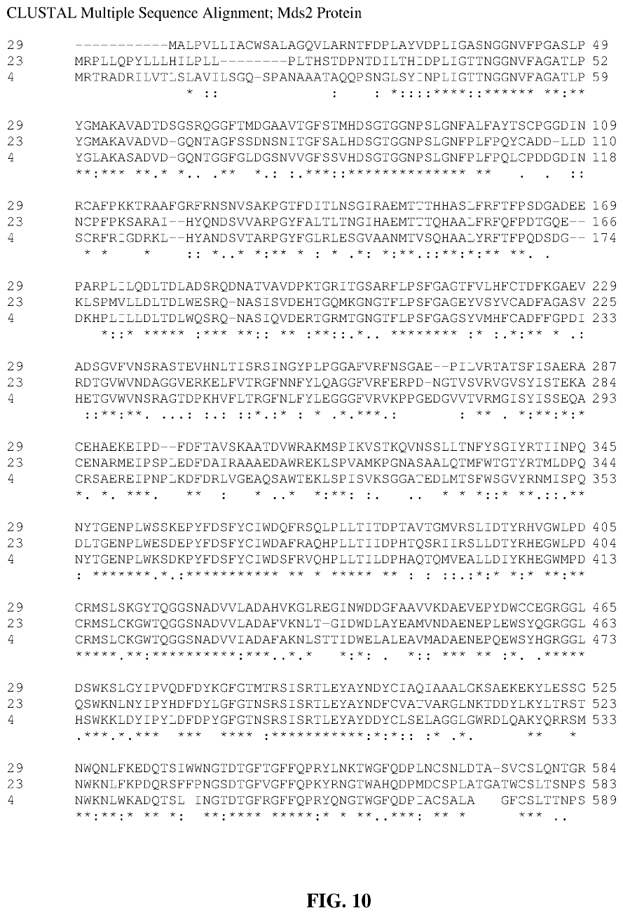

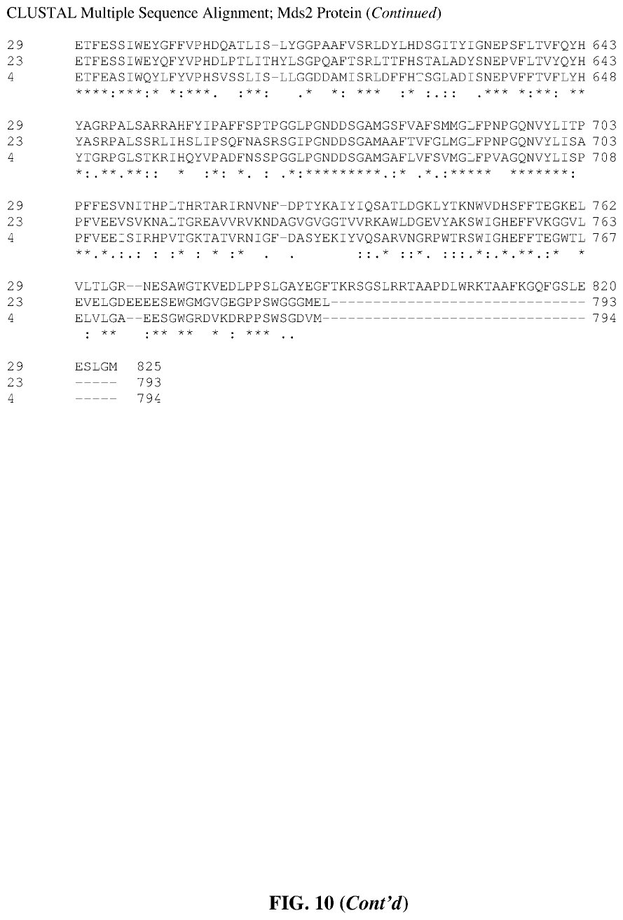

More particularly, as described in Example 4, (see, FIG. 6, FIG. 8, and FIG. 10), the A. niger and T. thermophilus GIIα, Mds1 and Mds2 protein homologues have substantial sequence homology to the T. reesei GIIα, Mds1 and Mds2 proteins, respectively. For instance, the A. niger genes encoding the Mds1 (SEQ ID NO: 21) and Mds2 (SEQ ID NO: 23) protein homologues comprise about 51% and 60% amino acid sequence identity to the T. reesei Mds1 (SEQ ID NO: 2) and Mds2 (SEQ ID NO: 4) proteins, respectively.

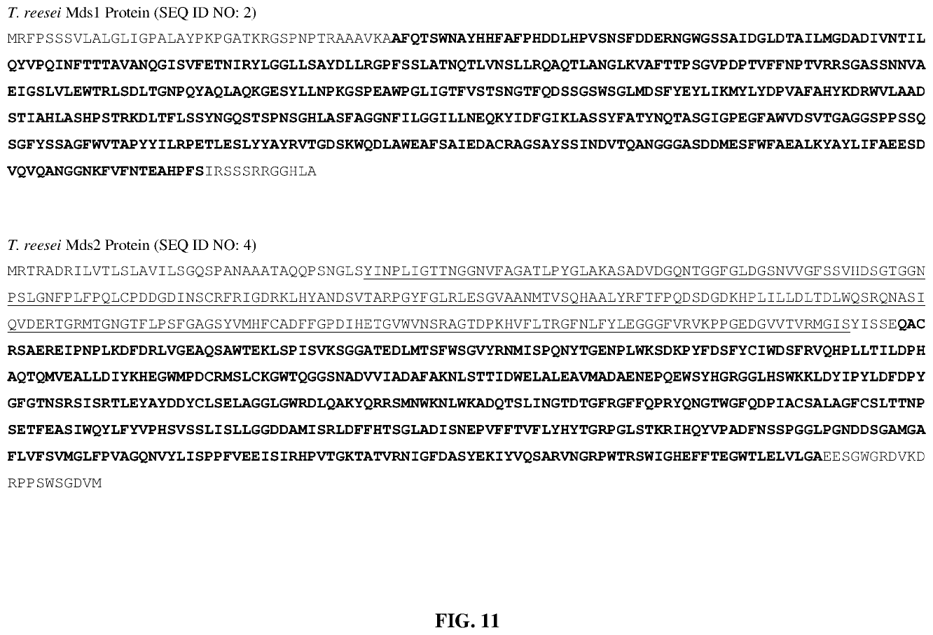

Thus, in one or more embodiments of the disclosure, by reference to the specification. Examples 1-4, and FIG. 5-FIG. 11, one of skill in the art may readily construct modified filamentous fungal strains deficient in the production of one or mannosidases set forth and described herein. For example, as generally shown in FIG. 11, the T. reesei Mds1 protein comprises a total of 523 amino acid residues, wherein amino acid residues from about position 43 to position 511 of SEQ ID NO: 2 are indicated with bold residues. In particular, amino acid positions of about 43 to about position 511 set forth in SEQ ID NO: 2 (FIG. 11) comprise a glycosyl hydrolase family 47 (GH47) sequence domain, wherein members of this family are alpha-mannosidases that catalyze the hydrolysis of the terminal 1,2-linked alpha-D-mannose residues.

Likewise, as presented in FIG. 11, the T. reesei Mds2 protein comprises a total of 794 amino acid residues, wherein amino acid residues from about position 39 to position 286 of SEQ ID NO: 4 are indicated with underlined residues, and amino acid residues positions from about position 292 to about position 773 of SEQ ID NO: 4 are indicated with bold residues. More particularly, as shown in FIG. 11 (SEQ ID NO: 4), amino acid positions of about position 39 to about position 286 comprise an N-terminal glycosyl hydrolase family 92 (GH92) sequence domain, and amino acid positions of about position 292 to about position 773 comprise a glycosyl hydrolase family 92 sequence domain, wherein members of this family are alpha-1,2-mannosidases, enzymes which remove alpha-1,2-linked mannose residues.

In certain embodiments, a gene or gene homologue encoding a mannosidase comprising amino acid sequence homology to the Mds1 protein of SEQ ID NO: 2 is modified herein to render the strain deficient in the production of the Mds1 protein (or homologue thereof) and/or a gene or gene homologue encoding a mannosidase comprising amino acid sequence homology to the Mds2 protein of SEQ ID NO: 4 is modified herein to render the strain deficient in the production of the Mds2 protein (or homologue thereof).

In certain embodiments, one or more genes (or genetic elements such as promoters, gene coding sequences, 5′-UTRs, terminators, etc.) encoding one or more mannosidases are genetically modified (e.g., via (a) the introduction, substitution, or removal of one or more nucleotides in the gene, or introduction, substitution, or removal of one or more nucleotides in a regulatory element required for the transcription or translation of the gene, (b) a gene disruption. (c) a gene conversion, (d) a gene deletion, (e) the down-regulation of a gene, (f) specific mutagenesis and/or (h) random mutagenesis of any one or more the genes disclosed herein, and the like. For example, one of skill may mutagenize, disrupt, delete or interfere with (e.g., RNAi) a portion of the gene encoding the Mds1 protein's GH47 (family) sequence domain (FIG. 11, bold residues; or the entire GH47 domain), thereby reducing or completely eliminating production of the functional Mds1 protein. Similarly, one of skill may mutagenize, disrupt, delete or interfere with a portion of the gene encoding the Mds2 protein's GH92 (family) N-terminal sequence domain (FIG. 11, underlined residucs; or the entire GH92 N-terminal sequence domain) and/or the GH92 (super-family) sequence domain (FIG. 11, bold residues) thereby reducing or completely eliminating production of the functional Mds2 protein. In other embodiments, one of skill may mutagenize, disrupt, delete, and the like a portion of the gene encoding the Mds1 protein's (GH47; glycosyl hydrolase) active site, and/or the Mds2 protein's (GH92; glycosyl hydrolase) active site, thereby hereby reducing or completely eliminating production of the functional Mds1 and/or Mds2 proteins.

### IV. Proteins of Interest

As stated above, certain embodiments are related to genetically mutant and/or modified (recombinant) fungal cells comprising genetic modifications which express a gene encoding a protein of interest (POI). More particularly, certain embodiments are related to compositions and methods for the expression/production of such proteins of interest in the modified (mutant) fungal cells of the disclosure. Thus, in certain embodiments, recombinant fungal cells produced enhanced amounts of proteins of interest, including, but not limited to, enzymes, antibodies, receptor proteins, animal feed proteins, human food proteins protein biologics and the like.

In certain embodiments, proteins of interest are encoded/expressed/produced by endogenous filamentous fungal genes, such as for example, endogenous genes encoding cellulases, endoglucanase, xylanases, and the like. In certain other embodiments, proteins of interest are encoded/expressed/produced by heterologous polynucleotides encoding proteins if interest. In certain embodiments, protein of interest includes glycosylated proteins (glycoproteins).

In certain embodiments, a protein of interest is an enzyme selected from the group consisting of cellulases, hemicellulases, xylanases, peroxidases, proteases, lipases, phospholipases, esterases, cutinases, polyesterases, phytases, pectinases, keratinases, reductases, oxidases, phenol oxidases, lipoxygenases, ligninases, pullulanases, tannases, pentosanases, mannanases, α-glucanases. β-glucanases, hyaluronidases, chondroitinases, laccases, amylases, glucoamylases, acetyl esterases, aminopeptidase, arabinases, arabinosidases, arabinofuranosidases, carboxypeptidases, catalases, nucleases, deoxyribonucleases, ribonucleases, epimerases, α-galactosidases, β-galactosidases, glucan lysases, endo-β-glucanases, glucose oxidases, glucuronidases, invertases, and isomerases.

In certain embodiments, a POI is selected from an Enzyme Commission (EC) Number selected from the group consisting of EC 1, EC 2, EC 3, EC 4, EC 5, and EC 6.

Optimal conditions for the production of the proteins will vary with the choice of the host cell, and with the choice of the protein(s) to be expressed. Such conditions may be readily ascertained by one skilled in the art through routine experimentation and/or optimization.

The protein of interest can be purified or isolated after expression. The protein of interest may be isolated or purified in a variety of ways known to those skilled in the art depending on what other components are present in the sample. Standard purification methods include, but are not limited to, electrophoretic, molecular, immunological, and chromatographic techniques, including ion exchange, hydrophobic, affinity, and reverse-phase HPLC chromatography, and chromatofocusing. For example, the protein of interest may be purified using a standard anti-protein of interest antibody column. Ultrafiltration and diafiltration techniques, in conjunction with protein concentration, are also useful. The degree of purification necessary will vary depending on the intended use of the protein of interest. In certain instances, no purification of the protein will be necessary.

In certain other embodiments, to confirm that a genetically modified fungal cell of the disclosure produces an increased level of a protein of interest, various methods of screening may be performed. In certain embodiments, the protein of interest may be detected by its activity (e.g., enzymatic activity, binding activity, etc.) or chromatographic profile. In other embodiments, an expression vector may encode a polypeptide fusion to the target protein which serves as a detectable label or the target protein itself may serve as the selectable or screenable marker. The labeled protein may be detected via western blotting, dot blotting (methods available at the Cold Spring Harbor Protocols website), ELISA, or, if the label is GFP, whole cell fluorescence and/or FACS. For example, a 6-histidine tag would be included as a fusion to the target protein, and this tag would be detected by western blotting. If the target protein expresses at sufficiently high levels, SDS-PAGE combined with Coomassie/silver staining, may be performed to detect increases in variant host cell expression over parental (control) cell, in which case no label is necessary. In addition, other methods may be used to confirm the improved level of a protein of interest, such as, the detection of the increase of protein amount per cell or protein amount per milliliter of fermentation medium using HPLC methods of protein separation or standard total protein measurements based on Coomassie Blue or BCA Reagents.

The detection of specific productivity is another method to evaluate the protein production. Specific productivity (Qp) can be determined by the following equation:

$$Qp = g P / gDCW \cdot hr$$

wherein “gP” is grams of protein produced in the tank, “gDCW” is grams of dry cell weight (DCW) in the tank, “hr” is fermentation time in hours from the time of inoculation, which include the time of production as well as growth time. Ultimately, if a protein of interest has enzymatic activity, its level of expression can be calculated from enzymatic assay.

In certain other embodiments, modified filamentous fungal cells exhibit an increased total protein yield, wherein total protein yield is defined as the amount of protein produced (g) per gram of carbohydrate fed, relative to the (unmodified) parental strain. Thus, as used herein, total protein yield (g/g) may be calculated using the following equation:

$$Yf = Tp / Tc$$

wherein “Yf” is total protein yield (g/g), “Tp” is the total protein produced during the fermentation (g) and “Tc” is the total carbohydrate (g) fed during the fermentation (bioreactor) run. In certain embodiments, the increase in total protein yield of a modified strain (i.e., relative to a control strain) is an increase of at least about 0.1%, at least about 1%, at least about 5%, at least about 6%, at least about 7%, at least about 8%, at least about 9%, or at least about 10% or more as compared to the unmodified (parental) cell.

Total protein yield may also be described as carbon conversion efficiency/carbon yield, for example, as in the percentage (%) of carbon fed that is incorporated into total protein. Thus, in certain embodiments, a modified filamentous fungal cell comprises an increased carbon conversion efficiency (e.g., an increase in the percentage (%) of carbon fed that is incorporated into total protein), relative to the (unmodified) parental strain. In certain embodiments, the increase in carbon conversion efficiency of the modified strain (i.e., relative to the control strain) is an increase of at least about 0.1%, at least about 1%, at least about 5%, at least about 6%, at least about 7%, at least about 8%, at least about 9%, or at least about 10% or more as compared to the unmodified (parental) cell.

### V. Fermentation

Certain embodiments are related to compositions and methods for producing a protein of interest comprising growing, cultivating, or fermenting a modified (mutant) filamentous fungal cell of the disclosure. In general, fermentation methods well known in the art are used to ferment the fungal cells. In some embodiments, the fungal cells are grown under batch, fed batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation occurs without the addition of any components to the system. Typically, a batch fermentation qualifies as a “batch” with respect to the addition of the nutrients, while factors such as pH and oxygen concentration are controlled. The broth and culture compositions of the batch system change constantly up to the time the fermentation is stopped. Within batch cultures, cells progress through a static lag phase to a high growth log phase and finally to a stationary phase, where growth rate is diminished or halted. If untreated, cells proceed to apoptosis and eventually die. In general, in the batch phase, the bulk of the production of product occurs during the log phase.

A suitable variation on the standard batch system is the “fed-batch fermentation” system. In this variation of a typical batch system, after the log phase is finished, the substrate is added in increments as the fermentation progresses. Fed-batch systems are often used to avoid catabolite repression. Continuous feeding of the substrate allows the process to keep its concentration below critical level that could lead to inhibition of cellular metabolism and protein production. Batch and fed-batch fermentations are common and well known in the art.

Continuous fermentation is a system where a defined fermentation medium is added continuously to a bioreactor, and an equal amount of conditioned medium is removed simultaneously for processing. Continuous fermentation generally maintains the cultures at a constant (high) density, where cells are primarily kept in log phase growth. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Certain embodiments of the instant disclosure are related to fermentation procedures for culturing fungi. Fermentation procedures for production of cellulase enzymes are known in the art. For example, cellulase enzymes can be produced either by solid or submerged culture, including batch, fed batch and continuous-flow processes. Culturing is generally accomplished in a growth medium comprising an aqueous mineral salts medium, organic growth factors, a carbon and energy source material, molecular oxygen, and, of course, a starting inoculum of the filamentous fungal host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and certain trace elements such as copper, manganese, molybdenum, zinc, iron, boron, and iodine, and others, again in suitable soluble assimilable form, all as known in the art.

The fermentation process can be an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for filamentous fungi such as Trichoderma reesei, the temperature generally will be within the range of about 20° C. to 40° C., generally preferably in the range of about 25° C. to 34° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations and can be employed by bubbling through the aqueous ferment (fermentation medium) in suitable amounts. At the same time, such ammonia can also be employed to assist in pH control.

The pH range in the aqueous microbial ferment should be in the exemplary range of about 2.0 to 10.0. With filamentous fungi, the pH normally is within the range of about 2.5 to 8.0; with Trichoderma reesei, the pH normally is within the range of about 3.0 to 7.0. Preferences for pH range of microorganisms are dependent on the media employed to some extent, as well as the particular microorganism, and thus can be somewhat adjusted as can be readily determined by those skilled in the art.

Preferably, the fermentation is conducted in such a manner that the carbon-containing substrate can be controlled as a limiting factor, thereby providing good conversion of the carbon-containing substrate to products and avoiding contamination of the cells with a substantial amount of unconverted substrate. The latter is not a problem with water-soluble substrates since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

The fermentation can be conducted as a batch or continuous operation, fed batch operation is much to be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain maximal production rates and/or maximum yields.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### VI. Broth Conditioning and Protein Recovery Processes

As generally described above, certain embodiments of the disclosure are related to cultivating (fermenting) filamentous fungal cells for the expression/production/secretion of proteins (or glycoproteins) of interest. Certain embodiments are therefore related to fermentation broths obtained by fermenting filamentous fungal cells expressing and secreting proteins into the broth. In certain other one or more embodiments, fermentation broths comprising glycoproteins of interest are subjected to one or more protein recovery processes. In certain embodiments, the disclosure provides methods for recovering proteins from fungal cell fermentation broths, such as obtaining and collecting a filamentous fungal cell fermentation broth comprising a protein of interest, performing filtration processes to remove fungal cells, performing diafiltration processes to reduce the levels of free sugars, heat treating the broths at about 40° C. for a sufficient amount of time, recovering proteins from the broth and the like, wherein the recovered proteins comprise reduced levels of glycation. In related embodiments, proteins expressed, secreted and recovered from the broth retain higher levels of enzymatic activity during storage at about room temperature (e.g., about 20° C.-22° C.) and/or comprise enhanced thermal stability.

More particularly, as described herein, proteins are expressed and secreted into the fermentation broth, wherein the end of fermentation (EOF) broth is subjected to one or more protein recovery processes (steps), such as cell separation processes, protein concentration processes, protein purification processes, and the like. The recovery of proteins from a fermentation broth can be done by procedures known to one of skill in the art to obtain a desired protein preparation. For example, the broth will generally contain cellular debris, including cells, various suspended solids, and other biomass contaminants, as well as the desired protein(s) of interest. One of skill in the art is aware of suitable protein recovery processes including, but not limited to, conventional solid-liquid separation techniques (e.g., centrifugation, filtration, dialysis, microfiltration, rotary vacuum filtration), and other known processes to produce a cell-free filtrate. The terms “cell separation” or “cell separation process” are not meant to be limiting, and include any methods of cell separation and/or broth clarification known to those skilled in the art.

Likewise, the terms “concentration” or “concentration process” are not meant to be limiting and include concentration methods known to those skilled in the art, such as ultrafiltration, evaporation, centrifugation, and the like. It may be preferable to further concentrate the fermentation broth or the cell-free filtrate prior to crystallization using techniques such as ultrafiltration, evaporation, or precipitation. Precipitating the proteinaceous components of the supernatant or filtrate may be accomplished by means of a salt, e.g., ammonium sulfate, followed by purification by a variety of chromatographic procedures, e.g., ion exchange chromatography, affinity chromatography or similar art recognized procedures.

Thus, as generally set forth above, protein preparations, glycoprotein preparations and the like according to the instant disclosure may be recovered, purified, enriched and the like using methods known to one skilled the art (e.g., art-recognized separation techniques such as ion exchange chromatography, affinity chromatography, hydrophobic separation, dialysis, protease treatment, ammonium sulphate precipitation (or other protein salt precipitation), centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

Thus, in one or more preferred embodiments of the disclosure, a fermentation broth comprising one or more proteins of interest is collected. In related embodiments, the end of fermentation broth is collected (harvested) and subjected to one or more recovery processes which includes at least one heat treatment step. In certain embodiments, a heat treatment process is performed for at least about one (1) hour to about five (5) hours. In related embodiments, the heat treatment process is performed at temperature between about 38.5° C. to about 41.5° C. In certain other one or more embodiments, the heat treatment process comprises heat treating the broth for about four (4) hours at 40° C., and subsequently collecting the broth. In certain embodiments, the broth may be cooled to about room temperature (20° C.) or lower.

### VII. Exemplary Embodiments

Non-limiting embodiments of the disclosure include, but are not limited to:

1. A modified filamentous fungal cell derived from a parental cell comprising an endogenous gene encoding a functional alpha-mannosidase protein, wherein the modified cell comprises a genetic modification rendering the cell deficient in production of the functional alpha-mannosidase protein.

2. A modified filamentous fungal cell derived from a parental cell comprising endogenous genes encoding at least two functional alpha-mannosidase proteins, wherein the modified cell comprises genetic modifications rendering the cell deficient in the production of the at least two functional alpha-mannosidase proteins.

3. The modified cell of embodiment 1 or embodiment 2, wherein the endogenous gene or genes encode a functional alpha-mannosidase protein comprising at least about 50% to 100% identity to an amino acid sequence selected from the group consisting of SEQ ID NO: 2, SEQ ID NO: 4, SEQ ID NO: 21, SEQ ID NO: 23, SEQ ID NO: 27 and SEQ ID NO: 29.

4. The modified cell according to any one of embodiments 1-3, comprising one or more endogenous genes encoding one more proteins of interest and/or comprising one or more heterologous genes encoding one or more heterologous proteins of interest.

5. The modified cell according to embodiment 1 or embodiment 2, wherein the functional alpha-mannosidase protein selected from the group consisting of SEQ ID NO: 2, SEQ ID NO: 21 and SEQ ID NO: 27 comprises a glycosyl hydrolase family 47 (GH47) sequence domain.

6. The modified cell according to embodiment 1 or embodiment 2, wherein the functional alpha-mannosidase protein selected from the group consisting of SEQ ID NO: 4, SEQ ID NO: 23 and SEQ ID NO: 29 comprises an N-terminal glycosyl hydrolase family 92 (GH92) sequence domain and a GH92 superfamily sequence domain.

7. The modified cell of embodiment 1 or embodiment 2, wherein the genetic modification rendering the cell deficient in production of the functional alpha-mannosidase protein is selected from the group consisting of (a) the introduction, substitution or removal of one or more nucleotides in a gene encoding a functional alpha-mannosidase protein, and/or the introduction, substitution or removal of one or more nucleotides in a regulatory element required for the transcription or translation of the gene encoding a functional alpha-mannosidase protein, (b) an alpha-mannosidase gene disruption, (c) an alpha-mannosidase gene conversion, (d) an alpha-mannosidase gene deletion and (e) the down-regulation of an alpha-mannosidase gene.

8. The modified cell of embodiment 7, wherein the genetic modification is selected from the group consisting of a non-functional GH47 protein family sequence domain, a non-functional GH47 protein family active site, a non-functional N-terminal GH92 protein family sequence domain, a non-functional GH92 superfamily sequence domain, and a non-functional GH92 protein family active site.

9. The modified cell of embodiment 4, fermented under suitable conditions for production and secretion of the one or more proteins into the fermentation broth.

10. The modified cell of embodiment 9, wherein the end of fermentation (EOF) broth is collected and stored at room temperature for at least 4 hours to about 5 days.

11. The modified cell of embodiment 10, wherein the stored broth comprises a reduced amount of mannose (Man) sugars relative to the amount of Man sugars present in the EOF broth of the parental cell when collected and stored under the same conditions.

12. The modified cell of embodiment 9, wherein a heat treatment step is performed on the end of fermentation (EOF) broth, wherein the heat treated broth comprises a reduced amount of mannose (Man) sugars relative to the amount of Man sugars present in the EOF broth of the parental cell producing the same one or more proteins when fermented and heat treated under the same conditions as the modified cell.

13. The modified cell of any one of embodiments 9-12, wherein the one or more proteins are recovered from the broth.

14. The modified cell of any one of embodiments 9-12, wherein the modified and parental cells are fermented for at least about 96 to about 300 hours under the same conditions.

15. The modified cell according to any one of embodiments 1-14, selected from the group consisting of an Aspergillus sp. cell, an Emericella sp. cell, a Fusarium sp. cell, a Humicola sp. cell, a Mucor sp. cell, a Myceliophthora sp. cell, a Neurospora sp. cell, a Penicillium sp. cell, a Scytalidium sp. cell, a Thielavia sp. cell, a Tolypocladium sp. cell and a Trichoderma sp. cell

16. The modified cell of any one of embodiments 1-14, wherein the parental cell is a Trichoderma reesei cell comprising a mutated glucosidase IIa (gls2a^{Stop}) allele encoding a truncated glucosidase II α-subunit (GIIα^{Stop}) protein, wherein the modified a T. reesei cell comprises a restored glucosidase Ila (gls2a^{R}) allele encoding a native glucosidase II α-subunit (GIIα) protein.

17. The modified cell of embodiment 16, wherein the parental cell is a T. reesei strain selected from the group consisting of Rut-C30, RL-P37, NG14 strain, or an ancestor strain derived therefrom comprising mutated gls2a^{Stop} allele.

18. The modified cell of embodiment 13, wherein one or more proteins of interest produced comprise a homogenous N-linked glycan pattern comprising greater than about 75% Man5GlcNAc2 as compared to the N-linked glycan pattern of the same one or more proteins of interest produced by the parental cell, wherein the modified and parental cells are fermented under the same conditions and the one or more proteins of interest are recovered under the same conditions.

19. The modified cell of embodiment 4, wherein the one or more endogenous or heterologous genes encode a protein selected from the group consisting of a lipase, a glucoamylase and a phytase.

20. The modified cell of embodiment 16, wherein the modified cell produces an increased amount of a protein relative to the parental cell when fermented under the same conditions for the production of the protein.

21. A modified Trichoderma reesei cell derived from a parental T. reesei cell comprising a mutant glucosidase IIa (gls2a^{Stop}) allele encoding a truncated glucosidase II α-subunit (GIIα^{Stop}) protein, wherein the modified cell comprises a restored glucosidase IIα (gls2a^{R}) allele encoding a native glucosidase II α-subunit (GIIα).

22. The modified cell of embodiment 21, producing one or more endogenous proteins of interest and/or producing one or more heterologous proteins of interest.

23. The modified cell of embodiment 22, wherein the one or more proteins of interest are glycoproteins.

24. The modified cell of embodiment 23, wherein the one or more proteins of interest comprise a homogenous N-linked glycan pattern comprising greater than about 75% Man5GlcNAc2 as compared to the N-linked glycan pattern of the same one or more proteins of interest produced by the parental cell, wherein the modified and parental cells are fermented under the same conditions and the one or more proteins of interest are recovered under the same conditions.

25. The modified cell of embodiment 22, wherein the modified cell produces an increased amount of a protein of interest relative to the parental cell when fermented under the same conditions for the production of the protein of interest.

26. The modified cell of embodiment 21, wherein the parental cell is a T. reesei strain selected from the group consisting of Rut-C30, RL-P37, NG14 strain, or an ancestor strain derived therefrom comprising mutated gls2a^{Stop} allele.

27. The modified cell of embodiment 21, further comprising a genetic modification rendering the cell deficient in production of a functional alpha-mannosidase protein, or comprising a genetic modification rendering the cell deficient in production of at least two functional alpha-mannosidase proteins.

28. The modified cell of embodiment 27, fermented under suitable conditions for production and secretion of the one or more proteins into the fermentation broth.

29. The modified cell of embodiment 28, wherein the end of fermentation (EOF) broth is collected and stored at room temperature for at least 4 hours to about 5 days.

30. The modified cell of embodiment 29, wherein the stored broth comprises a reduced amount of mannose (Man) sugars relative to the amount of Man sugars present in the EOF broth of the parental cell when collected and stored under the same conditions.

31. The modified cell of embodiment 28, wherein a heat treatment step is performed on the end of fermentation (EOF) broth, wherein the heat treated broth comprises a reduced amount of mannose (Man) sugars relative to the amount of Man sugars present in the EOF broth of the parental cell producing the same one or more proteins when fermented and heat treated under the same conditions as the modified cell.

32. The modified cell of embodiment 31, wherein the one or more proteins are recovered from the treated broth.

33. The modified cell of embodiment 28, wherein the modified and parental cells are fermented for at least about 96 to about 300 hours under the same conditions.

34. The modified cell of embodiment 22, wherein the one or more proteins of interest are selected from the group consisting of lipases, glucoamylases and phytases.

35. A method for fermenting a filamentous fungal cell for the production and recovery of a protein of interest (POI) in a fermentation broth comprising a reduced amount of mannose (Man) sugars, the method comprising obtaining a parental cell comprising an endogenous gene encoding a functional alpha-mannosidase protein and genetically modifying the cell to be deficient in the production of the functional alpha-mannosidase protein and fermenting the modified cell under suitable conditions for production of the POI, wherein the end of fermentation (EOF) broth of the modified cell comprises a reduced amount of Man sugars relative to the EOF broth of the parental cell fermented under the same conditions.

36. A method for fermenting a filamentous fungal cell for the production and recovery of a protein of interest (POI) in a fermentation broth comprising a reduced amount of mannose (Man) sugars, the method comprising obtaining a parental cell comprising at least two endogenous genes encoding at least two functional alpha-mannosidase proteins and genetically modifying the cell to be deficient in the production of the at least two functional alpha-mannosidase proteins and fermenting the modified cell under suitable conditions for production of the POI, wherein the end of fermentation (EOF) broth of the modified cell comprises a reduced amount of Man sugars relative to the EOF broth of the parental cell fermented under the same conditions.

37. The method of embodiment 35, wherein the endogenous gene encodes a functional alpha-mannosidase protein comprising at least about 50% to 100% identity to an amino acid sequence selected from the group consisting of SEQ ID NO: 2, SEQ ID NO: 4, SEQ ID NO: 21, SEQ ID NO: 23, SEQ ID NO: 27 and SEQ ID NO: 29.

38. The method of embodiment 36, wherein the least two endogenous gene encodes a functional alpha-mannosidase protein comprising at least about 50% to 100% identity to an amino acid sequence selected from the group consisting of SEQ ID NO: 2, SEQ ID NO: 4, SEQ ID NO: 21. SEQ ID NO: 23. SEQ ID NO: 27 and SEQ ID NO: 29.

39. The method of embodiment 35 or embodiment 36, wherein the POI produced is secreted into the fermentation broth.

40. The method of embodiment 35 or embodiment 36, wherein the filamentous fungal cell is selected from the group consisting of an Aspergillus sp. cell, an Emericella sp. cell, a Fusarium sp. cell, a Humicola sp. cell, a Mucor sp. cell, a Myceliophthora sp. cell, a Neurospora sp. cell, a Penicillium sp. cell, a Scytalidium sp. cell, a Thielavia sp. cell, a Tolypocladium sp. cell and a Trichoderma sp. cell

41. The method of embodiment 35 or embodiment 36, wherein the modified and parental cells are fermented for at least about 96 to about 300 hours under the same conditions.

42. The method of embodiment 35 or embodiment 36, wherein the EOF broth is subjected to a heat treatment process (step).

43. The method of embodiment 42, wherein the heat treated EOF broth from the modified cell comprises a reduced amount of Man sugars relative to the EOF broth of the parental cell subjected to the same heat treatment process.

44. The method of embodiment 35 or embodiment 36, wherein the parental cell is a Trichoderma reesei cell comprising a mutated glucosidase IIα (gls2a^{Stop}) allele encoding a truncated glucosidase II α-subunit (GIIα^{Stop}) protein, wherein the modified a T. reesei cell comprises a restored glucosidase IIα (gls2a^{R}) allele encoding a native glucosidase II α-subunit (GIIα) protein.

45. The method of embodiment 44, wherein the POI recovered comprises a homogenous N-linked glycan pattern comprising greater than about 75% Man5GlcNAc2 as compared to the N-linked glycan pattern of the same POI produced by the parental cell, wherein the modified and parental cells are fermented under the same conditions.

46. The method of embodiment 44, wherein the modified cell produces an increased amount of the POI relative to the parental cell when fermented under the same conditions for the production of the POI.

47. The method of embodiment 44, wherein the parental cell is a T. reesei strain selected from the group consisting of Rut-C30, RL-P37, NG14 strain, or an ancestor strain derived therefrom comprising mutated gls2a^{Stop} allele.

48. A method for producing a protein of interest (POI) comprising a homogenous N-linked glycan pattern comprising (a) obtaining a Trichoderma reesei cell comprising a mutated glucosidase IIα (gls2a^{Stop}) allele encoding a truncated glucosidase II α-subunit (GIIα^{Stop}) protein and genetically modifying the cell to express and encode a restored glucosidase IIα (gls2a^{R}) allele encoding the native glucosidase II α-subunit (Gila) and fermenting the modified cell under suitable conditions for production and secretion of a POI, and recovering the POI from the fermentation broth, wherein the recovered POI comprises a homogenous N-linked glycan pattern comprising greater than 75% Man5GlcNAc2 as compared to the N-linked glycan pattern of the same POI produced by the parental cell, wherein the modified and parental cells are fermented under the same conditions and the POI recovered under the same conditions.

49. The method of embodiment 48, wherein the modified cell produces an increased amount of the POI relative to the parental cell when fermented under the same conditions for the production of the POI.

50. The method of embodiment 48, wherein the parental cell is a T. reesei strain selected from the group consisting of Rut-C30, RL-P37, NG14 strain, or an ancestor strain derived therefrom comprising mutated gls2a^{Stop} allele.

51. The method of embodiment 48, further comprising a genetic modification rendering the cell deficient in production of a functional alpha-mannosidase protein, or comprising a genetic modification rendering the cell deficient in production of at least two functional alpha-mannosidase proteins.

52. The method of embodiment 48, wherein modified cell secretes the POI into the fermentation broth.

53. The method of embodiment 52 wherein the POI is recovered from the broth.

54. The method of embodiment 48, wherein the modified and parental cells are fermented for at least about 96 to about 300 hours under the same conditions.

55. The method of any one of embodiments 35, 36, or 48, wherein the cells comprise one or more introduced expression constructs encoding the one or more heterologous proteins of interest.

58. The method of embodiment 55, wherein the one or more one or more expression constructs encode one or more proteins of interest are selected from the group consisting of lipases, glucoamylases and phytases.

### EXAMPLES

Certain embodiments of the present disclosure may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989).

### Example 1

### Genetic Modifications of Recombinant Fungal Cells Expressing Heterologous Glycoproteins

As generally set forth above, the Trichoderma strains described in the instant examples may be derived from publicly available strains (e.g., RutC-30, RLP37, etc.), which Trichoderma strains are well suited for the expression/production of endogenous cellulases and heterologous (recombinant) proteins of interest. More particularly, certain Trichoderma reesei strains of the instant example were derived from T. reesei strain RLP37 (i.e., comprising the mutated (frameshift) glsa2^{Stop} allele), after deletion of four native cellulase genes (cbh1, cbh2, egl1, egl2), as generally described in PCT Publication No. WO2005/001036 (incorporated herein by reference). Thus, in certain embodiments, Applicant has designed and constructed recombinant T. reesei cells/strains (e.g., strain RLP37) to express heterologous glycoproteins, including a phytase (glycoprotein) reporter, a glucoamylase (glycoprotein) reporter and a lipase (glycoprotein) reporter glycoprotein. More particularly, any suitable protein may be expressed/produced in one or more recombinant T. reesei cells of the disclosure, and further tested, screened, assayed and the like, as generally set forth and described herein. Thus, in certain embodiments of the disclosure. Applicant has screened three (3) exemplary glycoproteins in parental T. reesei cells.

In certain embodiments. Applicant screened a heterologous phytase reporter protein in a parental T. reesei strain (Phy) comprising the mutated glsa2^{Stop} allele and in a modified T. reesei strain (Phy-gls2a^{R}) producing the same heterologous phytase reporter protein and comprising the restored glsa2^{R} allele. In other embodiments. Applicant screened the same heterologous phytase reporter protein in a modified T. reesei strain (Phy-Δmds2) comprising a deleted mds2 allele. In other embodiments, Applicant screened a heterologous glucoamylase (GA) reporter protein in a parental T. reesei strain comprising the mutated glsa2^{Stop} allele (GA-glsa2^{Stop}) and in a modified T. reesei strain producing the same heterologous glucoamylase reporter protein and comprising the restored glsa2^{R} allele (GA-gls2a^{R}). In yet other embodiments. Applicant screened a heterologous lipase reporter protein in a parental T. reesei strain (Lip) and in a modified T. reesei strain (Lip-Δmds1) comprising a deleted mds1 allele.

More particularly, such heterologous phytase protein sequences and genes encoding the same are well known in the art, including, but not limited to, phytase and phytase variant sequences recited in PCT Publication No. WO2008/097619, PCT Publication No. WO2009/129489 and PCT Publication No. WO2013/119470 (incorporated herein by reference in its entirety). Likewise, such heterologous glucoamylase protein sequences and genes encoding the same are well known in the art, including, but not limited to glucoamylase and glucoamylase variant sequences recited in PCT Publication No. WO2021/212095 (incorporated herein by reference in its entirety). Likewise, such heterologous lipase reporter protein sequences and genes encoding the same are well known in the art, including, but not limited to, PCT Publication No. WO2020/190782 (incorporated herein by reference in its entirety).

As briefly set forth above, in certain other embodiments, to more directly compare data with a T. reesei strain producing endogenous cellulases (cbh1, cbh2, egl1, egl2), a parental cellulase reporter strain named “Cel” was constructed by re-introducing the cbh1, cbh2, egl1, egl2 genes encoding the native cellulases as a single integrated expression cassette.

In the following examples, parental T. reesei strains were modified as described herein, wherein the resulting transformant (modified) strains were screened microtiter plate (MTP) fermentations to confirm expression of cellulases or heterologous glycoproteins.

As set forth herein, all Trichoderma strains used in the following Examples comprise a deletion of the Endo T allele (SEQ ID NO: 36), wherein the Endo T allele encodes a secreted endo-N-acetyl-f-D-glucosaminidase (deglycosylating enzyme). For example, as generally described by Stals et al. (2012), the mannosyl glycoprotein endo-N-acetyl-β-D-glucosaminidase was shown to be responsible for the microheterogeneity observed for H. jecorina cellulases and hemicellulases, wherein deletion of the EndoT allele in H. jecorina eliminate this activity, thereby retaining larger N-linked glycan chains on secreted proteins.

### A. Gls2a Restoration

As briefly described above, the glsa2 allele in certain Trichoderma strains was determined to have a frameshift mutation at nucleotide position 1,965 resulting in aberrant processing of N-linked glycans. Such a phenotype may impact the production of recombinant glycoproteins. Based on the foregoing, Applicant has restored the mutated (frameshift) glsa2 allele (gls2a^{R}; SEQ ID NO: 7) with a restored glsa2 allele (gls2a^{R}; SEQ ID NO: 5), wherein the Trichoderma strains comprise an introduced phytase glycoprotein, an introduced glucoamylase glycoprotein, or an introduced cellulase reporter protein expression cassette (cbh1, cbh2, egl1, egl2) encoding lignocellulosic degrading enzymes Cbh1, Cbh2, Egl1, Egl2, respectively.

In particular, Cas9 nuclease and a custom synthetic guide RNA (named “RHG2”; SEQ ID NO: 9) were obtained from Synthego (Menlo Park, California). Cas9-sgRNA complexes were assembled in vitro following the manufacturer's protocol and used to transform Trichoderma strains as generally set forth in PCT Publication No. WO2016/100568 (incorporated herein by reference in its entirety). Specific editing of the gls2a frameshift was accomplished by including 200 picomole of a 90 base pair double-stranded donor DNA (SEQ ID NO: 10), assembled from two oligonucleotides. Transformant colonies were screened using PCR, the product(s) of which were Sanger sequenced (data not shown). While the donor fragment supplied the missing base to repair the frameshift, it also included a single base change to further ensure the donor fragment and the resulting repaired gene were no longer cleavable by Cas9. The base change was made in the wobble position to avoid altering the protein sequence. However, in some cases, this led to transformants that only contained the frameshift repair.

### B. Mds2 Deletion

As described above, the mds2 gene was deleted to assess its role in release of mannose from glycoproteins, cell wall oligosaccharides, and/or other materials during and after fermentation. In the instant example, Applicant deleted the T. reesei mds2 gene (SEQ ID NO: 3) in the phytase reporter strain described above. More particularly, Cas9 nuclease and custom synthetic guide RNAs LFP009 (SEQ ID NO: 11) and LFP010 (SEQ ID NO: 12) were obtained from Synthego (Menlo Park, California). Cas9-sgRNA complexes were formed following the manufacturer's protocol. Transformant colonies were screened using PCR, the product(s) of which were Sanger sequenced (data not shown). Deletions of mds2 resulting from non-homologous end joining (NHEJ), as well as oligo-mediated homologous recombination (HR oligos, LFP013 (SEQ ID NO: 13) and LFP014 (SEQ NO: 14), provided as a single-stranded mixture (100 picomole each) were both detected when co-transformed with a selectable marker targeted to other, unlinked loci.

### C. Mds1 Deletion

As described above, the mds1 gene was deleted to assess its role in release of mannose from glycoproteins, cell wall oligosaccharides, and/or other materials during and after fermentation. In the instant example, Applicant has deleted the T. reesei mds1 gene (SEQ ID NO: 1) in the lipase reporter strain described above. More particularly, Cas9 nuclease and custom guide RNAs TCg3 (SEQ ID NO: 15) and TCg4 (SEQ ID NO: 16) and tracrRNA were obtained from Synthego (Menlo Park, California). Cas9-sgRNA complexes were formed following the manufacturer's protocol. Deletions of mds1 resulting from non-homologous end joining (NHEJ) as well as oligo-mediated homologous recombination (HR oligo TC128; SEQ ID NO: 17), provided as a single-stranded oligonucleotide mixture (100 picomole) were both detected when co-transformed with a selectable marker targeted to other, unlinked loci. Transformant colonies were screened using PCR, the product(s) of which were Sanger sequenced (data not shown).

A summary of the strains constructed herein are set forth below in TABLE 1.

TABLE 1
TRICHODERMA STRAIN DESCRIPTIONS

| Strain Name | Strain Description |
|---|---|
| Phy (Parent) | Phytase expression cassette + gls2a^{Stop} |
| Phy-gls2a^{R} (Modified) | Phytase expression cassette + restored gls2a allele |
| Phy-Δmds2 (Modified) | Phytase expression cassette + deleted mds2 |
| | allele + gls2a^{Stop} |
| GA (Parent) | Glucoamylase expression cassette + gls2a^{Stop} |
| GA-gls2a^{R} (Modified) | Glucoamylase expression cassette + restored |
| | gls2a allele |
| Lip (Parent) | Lipase expression cassette + gls2a^{Stop} |
| Lip-Δmds1 (Modified) | Lipase expression cassette + deleted mds1 allele + |
| | gls2a^{Stop} |
| Cel (Parent) | Cellulase expression cassette (cbh + cbh2 + |
| | egl1 + egl2) + gls2a^{Stop} |
| Cel-gls2a^{R} (Modified) | Cellulase expression cassette + restored gls2a |
| | allele |

### D. Expression of Proteins in Microtiter Plates (MTP)

Transformant and control strains were cultured as generally described in Example 3 of International Application No. PCT/US2022/075210 (incorporated herein by reference in its entirety), except that preculturing was conducted in twenty-four (24) well plates (CytoOne, Catalog No. CC7672-754) for twenty-four (24) hours, production medium contained 2.5% (w/v) glucose/sophorose mixture and lactose slow release microtiter plates were incubated at 27° C. (five days). Ten (10) microliters diluted filtrate was analyzed by SDS-PAGE (NuPAGE system, following manufacturer's protocols). As presented in FIG. 3 (left panel; no treatment), the phytase glycoprotein reporter produced by the modified Phy-gls2a^{R} strain, as compared to the parental Phy strain, showed a small downward shift in apparent molecular weight (MW). As shown in FIG. 3 (right panel; w/Endo H treatment), after treatment with endoglycosidase H (EndoH, New England BioLabs) to remove the N-glycans, the protein band (MW) sizes did not differ between the Phy parental strain and the modified Phy-gls2a^{R} (restored glsa2 allele) strain. Likewise. SDS-PAGE analysis of the modified GA-gls2a^{R} and Cel-gls2a^{R} strains also showed a small downward shift as compared to the parental GA and Cel strains, respectively (data not shown). The protein mobility shift was indicative of smaller glycan chains, which was subsequently confirmed in Example 3, below.

MTP samples were additionally assayed for enzyme activities and protein titer. The phytase reporter was assayed using p-nitrophenyl phosphate substrate (Thermo Scientific cat no. 34045, 10 mM in sodium acetate buffer, pH 5.5). Activity was measured spectrophotometrically (absorbance at 405 nm) after a pH shift with 2N NaOH. The glucoamylase (GA) reporter was assayed by detection of glucose released in thirty (30) minutes from a maltodextrin solution (Sigma Aldrich Catalogue No. 419672) in sodium acetate buffer (pH 4.3). After thermal inactivation, glucose was measured spectrophotometrically using a D-glucose assay kit (K-GLUC, Megazyme, Bray, Ireland). As presented below in TABLE 2, higher activity titers were observed in the gls2a^{R} modified glucoamylase reporter strain (GA-gls2a^{R}) compared to the parent strain (GA), whereas the phytase reporter did not show a significant difference between the parent (Phy) and modified (Phy-gls2a^{R}) strain genotypes. These findings were confirmed in bioreactor experiments as described below.

TABLE 2
ENZYME ACTIVITY MEASURED IN
MTP SUPERNATANT SAMPLES

| Strain | Activity Signal |
|---|---|
| GA (Parent) | 0.41 +/− 0.02 (A510) |
| GA-gls2a^{R} (Modified)* | 0.55 +/− 0.09 (A510) |
| Phy (Parent) | 0.69 +/− 0.06 (A405) |
| Phy-gls2a^{R} (Modified)^{#} | 0.67 +/− 0.03 (A405) |

*Average of 22 isolates;
^{#}Average of 3 isolates

E. Expression of Protein in large-Scale Fermenters

As described herein, reporter proteins were expressed in large scale (~14L) bioreactors. These fermentations were conducted as generally described in PCT Publication No. WO2020/028126 (incorporated herein by reference in its entirety) with the glucose-sophorose feed stage conducted at 25° C. For the Phy, Lip and Cel reporters, modifications did not significantly alter glycoprotein titer or yield. However, restoration of gls2a in the GA reporter (GA-gls2a^{R}) resulted in significant increases in most fermentation performance metrics, as set forth below in TABLE 3. In particular, as shown in TABLE 3, protein yields, titers and specific productivity (Qp) were determined after about one-hundred forty (~140) hours of fermentation, wherein the modified strain (GA-gls2a^{R}) has an increased protein yield (62%), increased titer (53%) and increased Qp, as compared to the parental (GA) strain, respectively.

TABLE 3
BIOREACTOR PRODUCTION OF GLUCOAMYLASE
REPORTER PROTEIN

| Strain | Protein Yield | Protein Titer | Qp |
|---|---|---|---|
| GA (Parent) | 100% | 100% | 100% |
| GA-gls2a^{R} (Modified) | 162% | 153% | 202% |

### Example 2

### Large Scale Fermentation and Determination of Mannose Release During Glycoprotein Production and Recovery

As described herein, reporter proteins were expressed in large scale (~14L) bioreactors. Following the end of the fermentation period (about 200-300 hours), soluble proteins from the end of fermentation broth, including phytase and lipase reporters, were separated from the cells using standard protein recovery and processing methods known in the art. Following cell separation, diafiltration using standard approaches was used to reduce the concentration of small molecules including sugars. Following this recovery process, the diafiltered samples were split and one portion was heated at 40° C. for four (4) hours and then frozen at −20° C., whereas the other sample was immediately frozen at −20° C., wherein the amount of mannose in the samples before and after the final heat treatment step was measured using standard analytical procedures. In particular, the proteins were removed from samples by precipitation with acetone. The sugars were then separated by HPLC using a Waters Xbridge column and then quantified using a Thermo ISQ EM single quad mass spectrometer. Since genetic modifications were made in different parental strains (i.e., Phy, Lip and Cel parental strains), the mannose release from each modified strain was compared to its appropriate parental (control) strain.

For example, the results of the mannose release are presented below in TABLE 4, demonstrating that mutations of any of the three screened genes (i.e., Δmds1, Δmds2, and gls2a^{R}) are particularly suitable for reducing the amount of mannose released during recovery processes (e.g., at least about 7% to 40%), including heating steps. Thus, as demonstrated herein, such reductions in mannose release are particularly suitable in mitigating protein glycation events during the downstream processing, recovery and storage of such proteins produced in filamentous fungal strains.

TABLE 4
MANNOSE RELEASE BY HEAT TREATMENT FOLLOWING
STANDARD FERMENTATION AND RECOVERY FROM
PARENTAL AND MODIFIED STRAINS

| | | | | Heat- | Reduction in |
|---|---|---|---|---|---|
| | | Mannose in | Mannose | induced | heat-induced |
| | | unheated | in heated | increase in | mannose |
| | Strain | sample | sample | mannose | release due to |
| Strain Name | Description | (ug/ml) | (ug/ml) | (ug/ml) | mutation |
| Lip (Parent) | Lipase cassette | 902 | 1734 | 832 | NA |
| | (control) | | | | |
| Lip-Δmds1 (Modified) | Lip cassette w/ | 779 | 1552 | 773 | 7% |
| | mds1 deletion | | | | |
| Phy (Parent) | Phytase cassette | 97 | 1138 | 1041 | NA |
| | (control) | | | | |
| Phy-Δmds2 (Modified) | Phy cassette w/ | 68 | 693 | 625 | 40% |
| | mds2 deletion | | | | |
| Phy-gls2a^{R} (Modified) | Phy cassette w/ | 81 | 792 | 711 | 32% |
| | gls2a^{R} allele | | | | |

### Example 3

### Determination of N-Glycan Composition by Mass Spectrometry

In the instant example, proteins produced by the T. reesei phytase and cellulase reporter strains were characterized by protein and peptide mass spectrometry methods. Enzymatic digestion (Section A) allows the characterization of N-glycans at specific positions in the polypeptide sequence, whereas intact mass spectrometry (Section B) allows a quantitative assessment of overall glycosylation state (or states).

A. Detection of N-Glycans on Reporter Protein Peptides after Enzymatic Digestion

Supernatant samples from fermenters or microtiter plate cultures, obtained by centrifugation or filtration, were prepared for mass spectrometry using a filter-aided sample preparation method as follows. In a 1.7 ml Eppendorf tube, 200 μg protein was combined with 5 μl of 0.2 M dithiothreitol and incubated 30 minutes in an Eppendorf Thermo Mixer (50° C., 300 rpm). After addition of 10 μl of 440 mM iodoacetamide, the tube was incubated for an additional 30 minutes in the dark at room temperature. Protein was then precipitated with 0.5 ml acetone. After centrifugation (14,000 rpm, 10 minutes) the supernatant was removed, and the pellet allowed to dry for 5 minutes in a chemical hood. The pellet was resuspended in 150 μl 8 M urea (37° C., 5 minutes) and then filtered using Millipore Microcon-30 filters (Ultracel YM-30 regenerated cellulose 30.000 NMWL) at 14,000 rpm for 15 minutes. Ammonium bicarbonate (50 mM) was then applied three times, 100 μl each, with centrifugation (14,000 rpm, 10 minutes). The collection vial was replaced and ammonium bicarbonate (40 μl of 50 mM) and trypsin (20 μl of 0.1 mg/ml) were applied to the filter. This was allowed to incubate 12-18 hours at 37° C. (300 rpm). The filter was washed twice more with 50 mM ammonium bicarbonate (14,000 rpm, 10 minutes each) and the protein was eluted with 10 W of 0.1% formic acid (14,000 rpm, 10 minutes).

Protein digests were analyzed by Ultimate 3000 Nano LC/Thermo Q-Exactive HF. The analytical column for the Nano LC was a Thermo PepMap RSLC C18 column with column dimensions of 75 μm×50 cm, particle size of 2 μm and pore of 100 Å. Mobile phase A was 96% water, 4% acetonitrile and 0.1% formic acid. Mobile phase B was 20% water, 80% acetonitrile and 0.1% formic acid. The gradient profile was 4% B at 0-10 minutes, from 10 to 70 minutes, ramped % B to 45%; from 70.01 to 90 minutes, ramped % B to 99% and held at 99% B from 90 to 95 minutes. At 95.01 minutes, back to 4% B. The total run time was 120 minutes at a flowrate of 0.3 μl/min. MS/MS was performed on the Thermo Q-Exactive HF. Full mass scan resolution was 60,000. Scan range was 380-2000 m/z. AGC target was 1e6 and maximum IT was 150 ms. MS2 resolution was 15,000. Loop count was top 20, NCE was 30, fixed first mass was 100 m/z (AGC target was 1e5 and maximum IT was 100 ms). Glycosylation was analyzed with BioPharma Finder 3.5 software (ThermoFisher Scientific).

Data expressed as percent (%) relative abundance for phytase-derived peptides, and cellulase-derived peptides, are shown below in TABLE 5 and TABLE 6, respectively. For both the Phy and Cel proteins, restoration of gls2a (gls2a^{R}) resulted in nearly homogeneous Man5GlcNAc N-glycan content at each of the positions analyzed. For comparison, deletion of mds2, which was evaluated for the Phy reporter protein, which was found to decrease mannose release during downstream processing (Example 2), produced relatively minor changes in glycan composition in fermenter and MTP samples.

TABLE 5
DETECTION OF POST-TRANSLATIONAL GLYCAN MODIFICATIONS AT *ASN RESIDUES

| | Fermenter Samples | | | MTP Samples | | |
|---|---|---|---|---|---|---|
| Modification | Phy | Phy-Δmds2 | Phy-gls2a^{R} | Phy | Phy-Δmds2 | Phy-gls2a^{R} |
| N282 + GlcNAc | 2.7 | 6.4 | 5.4 | 1.1 | 1.7 | 0.5 |
| N282 + Man3GlcNAc | 0.2 | 0.1 | 0.4 | 0.0 | 0.0 | 0.1 |
| N282 + Man4GlcNAc | 2.5 | 1.7 | 4.7 | 0.3 | 0.1 | 1.9 |
| N282 + Man5GlcNAc | 47.4 | 39.5 | 93.2 | 25.4 | 17.7 | 98.3 |
| N282 + Hcx+6GlcNAc | 17.6 | 24.9 | 1.8 | 6.6 | 7.1 | 1.5 |
| N282 + Hex7GlcNAc | 8.3 | 12.9 | 0.4 | 3.9 | 3.3 | 0.1 |
| N282 + Hex8GlcNAc | 46.2 | 39.5 | 0.3 | 78.8 | 82.1 | 0.1 |
| N282 + Hex9GlcNAc | 2.2 | 2.2 | 0.0 | 1.7 | 2.2 | 0.0 |
| N282 + Unglycosylated | 9.6 | 10.5 | 8.3 | 0.5 | 0.6 | 0.2 |

*Asparagine (Asn) Residues are indicated by position number relative to the secreted protein.
#Mannose and Glucose have the same molecular masses. Glycan structures with mass higher than Man5GlcNAc in the Parent strains may correspond to monoglucosylated species described by Geysens et al (2005). Thus Hex6 = Glc.Man5, Hex7 = GlcMan6, etc.

TABLE 6
DETECTION OF POST-TRANSLATIONAL GLYCAN
MODIFICATIONS AT *ASN RESIDUES

| | | MTP Samples | | | |
|---|---|---|---|---|---|
| | | Cel | Cel | Cel- | Cel- |
| Protein | Modification | (Parent) | (Parent) | gls2a^{R} | gls2a |
| Cbh1 | N270 + GcNAc | 0.0 | 0.0 | 0.2 | 0.2 |
| | N270 + Man4GlcNAc | 0.1 | 0.1 | 0.8 | 0.8 |
| | N270 + Man5GlcNAc | 1.2 | 1.0 | 97.7 | 97.9 |
| | N270 + Hex^{≠}6GlcNAc | 4.3 | 4.0 | 1.3 | 1.1 |
| | N270 + Hex7GlcNAc | 16.9 | 17.9 | 0.1 | 0.1 |
| | N270 + Hex8GlcNAc | 65.0 | 63.6 | 0.0 | 0.0 |
| | N270 + Hex9GlcNAc | 13.0 | 13.9 | 0.0 | 0.0 |
| Cbh2 | N316 + Man5GlcNAc | 0.3 | 0.3 | 79.7 | 80.8 |
| | N316 + Hex6GlcNAc | 1.8 | 1.5 | 15.6 | 14.9 |
| | N316 + Hex7GlcNAc | 7.5 | 6.4 | 2.0 | 2.2 |
| | N316 + Hex8GlcNAc | 45.6 | 44.3 | 1.4 | 1.2 |
| | N316 + Hex9GlcNAc | 44.8 | 47.5 | 1.2 | 1.0 |

*Asparagine (Asn) Residues are indicated by position number relative to the secreted protein.
^{≠}Mannose and glucose have the same molecular masses.
Glycan structures with mass higher than Man5GlcNAc in the Parent strains may correspond to monoglucosylated species described by Geysens et al (2005).
Thus Hex6 = GlcMan5, Hex7 = GlcMan6, etc.

### B. Detection of N-Glycans on Intact Phytase Polypeptides

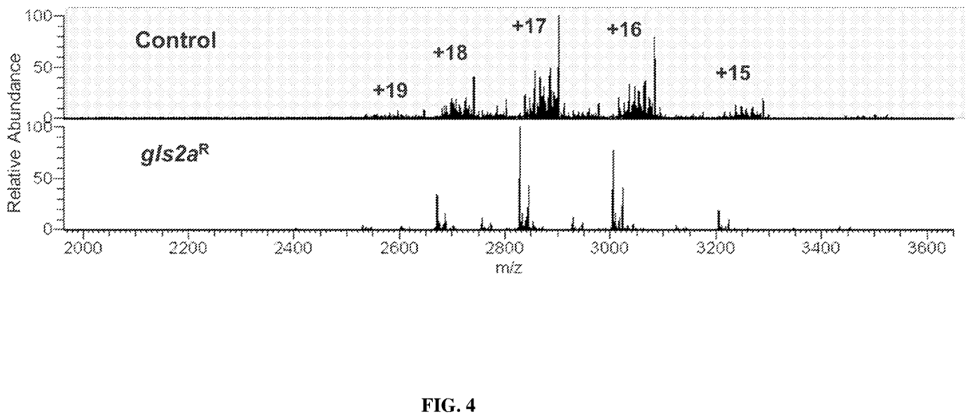

In the instant example, fermentation supernatant samples were diluted in 0.1% formic acid to achieve a concentration of less than 10 g/l. Samples were then analyzed using a Thermo Scientific™ Vanquish liquid chromatography system outfitted with a Waters BEH size exclusion column, which was coupled to a Thermo Scientific™ Q-Exactive Orbitrap HF mass spectrometer equipped with a HESI ion source. Protein separation was based on size by SEC using an isocratic mobile phase with 0.1% formic acid (aqueous). Mass spectrometric detection was achieved in positive ion ionization mode. The mass-to-charge (m/z) scans ranges varied depending on the charge state distributions observed for the proteins of interest (e.g. m/z 1800 to m/z 7000). The mass spectra resolution was set to 30,000 for the analysis of phytase samples. Thermo Scientific™ BioPharma Finder was used for data analysis. As presented in FIG. 4, the distribution of masses varying by mannose/hexose residues was found to be reduced in the gls2a^{R} fermentation samples as compared to the control strain.

### Example 4

### Deletion of Mds1 and Mds2 Genes in Other Filamentous Fungal Cells

As generally described above, in one or more embodiments of the disclosure, one or more mds1 and/or mds2 genes are genetically modified in one more filamentous fungal cells/strains of the disclosure. The instant example describes genetic modifications of mds1 and mds2 gene homologues in recombinant A. niger cells and T. thermophilus cells. For example, A. niger genes encoding proteins comprising about 51% and 60% amino acid sequence identity to the T. reesei Mds1 protein (FIG. 7 and FIG. 8: SEQ ID NO: 2) and Mds2 protein (FIG. 9 and FIG. 10; SEQ ID NO: 43) respectively were identified in the genome sequence of the public reference strain A. niger ATCC® 1015.

In particular, these sequences may be used to design synthetic guide RNAs named LFP028 (gRNA: SEQ ID NO: 30) and LYP029 (gRNA: SEQ ID NO:31) targeting the A. niger mds1 homolog (SEQ ID NO: 20), and gRNAs named LFP031 (gRNA: SEQ ID NO: 22) and LFP032 (gRNA; SEQ ID NO:34) targeting the A. niger mds2 homolog (SEQ ID NO: 22). For instance, Cas9 nuclease and custom synthetic guide RNAs described above may be obtained from suitable vendors, such as Synthego (Menlo Park. California). In certain embodiments, synthetic donor DNA may be included to provide a homology directed-repair template, including single or double stranded versions of DNA sequences LFP030 (SEQ ID NO: 32) and LFP033 (SEQ ID NO:35) for the mds1 and mds2 homologs, respectively. Transformants are obtained using unlinked selectable markers (e.g., hygromycin resistance, amdS, pyrG and the like).

In certain other embodiments, T. thermophilus genes encoding proteins comprising sequence homology to the T. reesei Mds1 protein (FIG. 7 and FIG. 8; SEQ ID NO: 2) and Mds2 protein (FIG. 9 and FIG. 10; SEQ ID NO: 4) have been identified in the genome sequence of the public reference strain T. thermophilus ATCC® 42464. As described above for the A. niger gene homologues, one of skill in the art may readily construct one or more gRNAs targeting the T. thermophilus mds1 and/or mds2 genes in a similar manner.

In other embodiments, one of skill in the art may readily construct modified filamentous fungal strains deficient in the production of one or mannosidases described herein. As shown in FIG. 11, the T. reesei Mds1 protein comprises 523 amino acid residues, wherein amino acid residues from about position 43 to position 511 of SEQ ID NO: 2 (bold residues) comprise a glycosyl hydrolase family 47 (GH47) sequence domain, wherein members of this family are alpha-mannosidases that catalyze the hydrolysis of the terminal 1,2-linked alpha-D-mannose residues. Furthermore, as shown FIG. 11, the T. reesei Mds2 protein comprises 794 amino acid residues, wherein amino acid residues from about position 39 to position 286 of SEQ ID NO: 4 (underlined residues) comprise an N-terminal glycosyl hydrolase family 92 (GH92) sequence domain, and amino acid residues positions from about position 292 to about position 773 of SEQ ID NO: 4 (bold residues) comprise a glycosyl hydrolase family 92 sequence domain, wherein members of this family are alpha-1,2-mannosidases, enzymes which remove alpha-1,2-linked mannose residues.

Based on the foregoing, the skilled artisan may readily design and construct one or more modified filamentous fungal strains deficient in the production of a Mds1 protein (or homologue thereof) and/or deficient in the production of a Mds2 protein (or homologue thereof). In particular, as described above, one or more genes (or genetic elements, e.g., promoters, gene coding sequences, 5′-UTRs, terminators, etc.) encoding one or more mannosidases are genetically modified herein, including, but not limited to, mutagenizing, disrupting, deleting, replacing, interfering with and the like, a portion of the gene encoding the Mds1 protein's GH47 (family) sequence domain, thereby reducing or completely eliminating production of the functional Mds1 protein. In other embodiments, one or more genes (or genetic elements) encoding one or more mannosidases are genetically modified herein, including, but not limited to, mutagenizing, disrupting, deleting, replacing, interfering with and the like, a portion of the gene encoding the Mds2 protein' GH92 (family) N-terminal sequence domain and/or GH92 super-family sequence domain, thereby reducing or completely eliminating production of the functional Mds2 protein. In other embodiments, one or more active site (amino acid) resides of the Mds1 and/or Mds2 protein are mutagenized, disrupted, deleted, and the like, thereby hereby reducing, or completely eliminating production of the functional Mds1 and/or Mds2 proteins.

### Example 5

### Assessing Reporter Protein Glycation and Activity

The instant example describes methods to assess one or more proteins produced by filamentous fungal cells of the disclosure. More particularly, as generally set forth above, certain embodiments are directed to modified filamentous fungal cells comprising genetic modifications rendering the cells deficient in production of a functional alpha-mannosidase protein and/or comprising a restored glucosidase IIα (gls2a^{R}) allele encoding a native glucosidase II α-subunit (GIIα). For example, as set forth above in Sections II and VI, modified filamentous fungal cells deficient in production of a functional alpha-mannosidase and/or having a restored gls2a^{R} allele may be cultivated/fermented under suitable conditions for the production of one or more proteins, wherein the proteins are recovered from the end of fermentation (EOF) broth. More specifically, after fermenting a modified fungal strain under suitable conditions for the production and secretion of the protein, the EOF broth may be processed as described in Example 2. For instance, after the EOF period (about 200-300 hours), secreted proteins are separated from the cells using standard protein recovery methods known to one of skill in in the art. Likewise, following such cell separation processes, standard diafiltration processes can be used to reduce the concentration of small molecules (e.g., sugars), wherein the diafiltered protein samples are subsequently heat treated for a sufficient amount of time and then frozen at −20° C.

In certain one or more embodiments of the disclosure, the modified filamentous fungal cells are fermented for at least about 180 hours to about 320 hours. In certain embodiments, modified filamentous fungal cells are fermented for at least about 200 hours to about 300 hours. In certain other embodiments, or embodiments, broth heat treatment processes are performed temperatures between about 39.5° C. to about 40.5° C. In certain embodiments, broth heat treatment processes are performed at temperatures of at least 40° C. In certain other embodiments, the heat treatment process is for period of time of about 30 minutes to about 4.5 hours. In related embodiments, the heat treatment process is a temperature of about 40° C. for about 4 hours.

Thus, following the above recovery and heat treatment processes, the samples are formulated using a composition which is appropriate for the particular protein and stored at 25° C. for 6 months, wherein aliquots of each sample are removed from storage once per month and the amount of free mannose (Man) is measured and the reporter protein analyzed for levels of glycation and/or protein (enzyme) activity and/or thermal stability. For example, Man levels may be measured as generally described above in Example 2. Likewise, as described herein, the glycation of a target protein (protein) may be measured by one of several techniques well-known in the art, including, but not limited to, changes in size of the protein measured by mass spectrometry (Schmitt et al., 2005(a)), the ultraviolet (UV) excited blue fluorescence emission from the glycated protein (Schmitt et al., 2005(b)), the characterization of lysine and arginine side chain modifications (Schmitt et al., 2005(b)) and the like.

In related embodiments, the activity of one or more proteins (enzymes) may be assayed using methods known to one of skill in the art. For example, phytase activity can be measured using p-nitrophenyl phosphate as a substrate (see, Example 1, Section D) and glucoamylase activity can be measured by the release of glucose from a maltodextrin solution (see, Example 1, Section D).

Lipase activity can be measured as described in PCT Publication No. WO2020/190782, using L-alpha-phosphatidylcholine (Avanti 441601G, Avanti Polar Lipids, USA) as a substrate dissolved in 50 mM HEPES buffer with 5 mM CaCl₂ using Triton-X 100 as emulsifier. For example, the amount of free fatty acid liberated during the enzymatic reaction can be measured using the NEFA kit (Wako Chemicals GmbH, Germany). Cellulase activity can be measured as described in Australian Patent Publication No. AU2016/200955 by measuring reducing sugars released from pretreated corn stover or phosphoric acid swollen cellulase. In other embodiments, thermal stability of the target protein can be measured by differential scanning calorimetry (DSC; Vetter and Indurthi, 2011). Thermal stability may also be assessed by the degree of enzyme activity (e.g., phytase, glucoamylase, lipase, cellulase, etc.) remaining after heat treating the samples for 10 minutes at temperatures of 40° C., 50° C., 60° C., 70° C., 75° C., 80° C., 85° C., 90° C., 95° C., and 98° C.

For example, one skilled in the art may perform one or more (several) of the above assays to assess the levels of free Man sugars in heat treated fermentation broth samples obtained from modified (recombinant) filamentous fungal cells comprising modifications rendering the cells deficient in production of a functional alpha-mannosidase protein and/or comprising a gls2a^{R} allele. Thus, any protein of interest may be expressed in one or more modified filamentous fungal cells of the disclosure and assessed as described herein. More particularly, by reference to one or more modified and parental strains exemplified below in TABLE 7, one of skill in the art may readily construct one or more modified filamentous fungal cells/strains for the production of other proteins of interest. For example, as contemplated and described herein, proteins of interest (e.g., phytases, glucoamylases, lipases, cellulases and the like) produced by one or more modified cells of the disclosure comprise reduced levels of glycation (i.e., as compared with their corresponding parents), retaining higher levels of protein (enzyme) activity during prolonged periods of storage at room temperature and comprising increased thermal stability.

TABLE 7
MEASUREMENT OF GLYCATION AND ENZYME STABILITY
FROM PARENTAL AND MODIFIED STRAINS

| Modified Allele | Modified Strain | Parental Strain | Reporter Protein |
|---|---|---|---|
| gls2a^{R} | GA-gls2a^{R} | GA-gls2a^{Stop} | Glucoamylase |
| gls2a^{R} | Phy-gls2a^{R} | Phy-gls2a^{Stop} | Phytase |
| gls2a^{R} | Cel-gls2a^{R} | Cel-gls2a^{Stop} | Cellulase |
| Δmds1 | Lip-Δmds1 | Lip-mds1 | Lipase |
| Δmds2 | Phy-Δmds2 | Phy-mds2 | Phytase |

### REFERENCES

Australian Patent Publication No. AU2016/200955
PCT Publication No. WO2003/038111
PCT Publication No. WO2006/040358
PCT Publication No. WO2008/097619
PCT Publication No. WO2009/129489
PCT Publication No. WO2011/153449
PCT Publication No. WO2013/119470
PCT Publication No. WO2020/028126
PCT Publication No. WO2020/190782
PCT Publication No. WO2021/212095
Ausubel et al., “Current Protocols in Molecular Biology”, Green Publishing Associates/Wiley Interscience, New York, Vol. 1 and Vol. 2, (1987; 1988, 1989).
Geysens et al., “Cloning and Characterization of the Glucosidase II Alpha Subunit Gene of Trichoderma reesei: a Frameshift Mutation Results in the Aberrant Glycosylation Profile of the Hypercellulolytic Strain Rut-C30”, Applied and Environmental Microbiology, Vol. 71, No. 6, pages 2910-2924, 2005.
Goto, “Protein O-Glycosylation in Fungi: Diverse Structures and Multiple Functions”, Biosci. Biotechnol. Biochem., 71, pages 1415-1427, 2007.
Knudsen et al., “FIND-IT: Accelerated trait development for a green evolution”, Sci. Adv. 8, eabg2266, 2022.
Kornfeld and Kornfeld, “Assembly of Asparagine-linked Oligosaccharides”, Ann. Rev. Biochem., 54, pages 631-664. 1985.
Le Crom et al. “Tracking the roots of cellulase hyperproduction by the fungus Trichoderma reesei using massively parallel DNA sequencing”, Proc. Natl. Acad. Sci. U.S.A., 106, pages 16151-16156, 2009
Sambrook et al., Molecular Cloning, A Laboratory Manual, 2^{nd} Edition, Cold Spring Harbor Laboratory Press, Cold Spring, New York, 1989.
Sambrook et al., Molecular Cloning, A Laboratory Manual, 4^{th} Edition, Cold Spring Harbor Laboratory Press, Cold Spring, New York, 2012.
Satoh et al., “Interaction mode between catalytic and regulatory subunits in glucosidase II involved in ER glycoprotein quality control”, Protein Science, Vol. 25, pages 2095-2101, 2016.
Schmitt et al., Analytical Biochemistry Volume 338, Issue 2, pages 201-215, 2005(b).
Schmitt et al., Analytical Biochemistry, Volume 346, Issue 1, pages 101-106, 2005(a)
Sheir-Neiss and Montenecourt, “Characterization of the secreted cellulases of Trichoderma reesei wild type and mutants during controlled fermentations”, Applied Microbiology and Biotechnology, 20(1):46-53, 1984.
Stals et al., “High Resolution Crystal Structure of the Endo-N-Acetyl-β-D-Glucosaminidase Responsible for the Deglycosylation of Hypocrea jecorina Cellulases”, PLoS ONE, Volume 7, Issue 7, 2012.
Sutthirak et al., “Effect of glycation on stability and kinetic parameters of thermostable glucoamylase from Aspergillus niger”, Process Biochemistry, Vol. 40, Issue 8, pages 2821-2926, 2005.
Vetter and Indurthi, “Moderate glycation of serum albumin affects folding, stability, and ligand binding”, Clinica Chimica Acta 412, pages 2105-2116, 2011.

1. A modified filamentous fungal cell derived from a parental cell comprising an endogenous gene encoding a functional alpha-mannosidase protein and having an introduced expression cassette encoding a protein of interest (POI), wherein the modified cell comprises a genetic modification rendering the cell deficient in production of the functional alpha-mannosidase protein.

2. The modified cell of claim 1, wherein the endogenous gene encodes a functional alpha-mannosidase protein comprising at least about 50% to 100% identity to an amino acid sequence selected from the group consisting of SEQ ID NO: 2, SEQ ID NO: 4, SEQ ID NO: 21, SEQ ID NO: 23, SEQ ID NO: 27 and SEQ ID NO: 29.

3. The modified cell of claim 1, fermented under suitable conditions for production and secretion of the POI into the fermentation broth.

4. (canceled)

5. (canceled)

6. The modified cell of claim 1, wherein the parental cell is a Trichoderma sp. cell comprising a mutated glucosidase IIα (gls2a^{Stop}) allele encoding a truncated glucosidase II α-subunit (GIIα^{Stop}) protein, wherein the modified a T. reesei cell comprises a restored glucosidase IIα (gls2a^{R}) allele encoding a native glucosidase II α-subunit (GIIα) protein.

7. The modified cell of claim 6, wherein the modified cell produces an increased amount of the POI relative to the parental cell when fermented under the same conditions for the production of the POI.

8. A modified Trichoderma reesei cell derived from a parental T. reesei cell comprising a mutant glucosidase IIα (gls2a^{Stop}) allele encoding a truncated glucosidase II α-subunit (GIIα^{Stop}) protein, wherein the modified cell comprises a restored glucosidase IIα (gls2a^{R}) allele encoding a native glucosidase II α-subunit (GIIα).

9. The modified cell of claim 8, producing one or more endogenous proteins of interest and/or producing one or more heterologous proteins of interest.

10. The modified cell of claim 9, wherein the modified cell produces an increased amount of the one or more proteins of interest relative to the parental cell when fermented under the same conditions for the production of the one or more proteins of interest.

11. (canceled)

12. The modified cell of claim 8, comprising a genetic modification rendering the cell deficient in production of a functional alpha-mannosidase protein, or comprising a genetic modification rendering the cell deficient in production of at least two functional alpha-mannosidase proteins.

13. A method for fermenting a filamentous fungal cell for the production and recovery of a protein of interest (POI) in a fermentation broth comprising a reduced amount of mannose (Man) sugars, the method comprising:
(a) obtaining a parent filamentous fungal cell comprising an endogenous gene encoding a functional alpha-mannosidase protein and introducing into the cell an expression cassette encoding a protein of interest (POI),
(b) genetically modifying the cell to be deficient in the production of the functional alpha-mannosidase protein, and
(b) fermenting the modified cell under suitable conditions for production of the POI, wherein the end of fermentation (EOF) broth from the modified cell comprises a reduced amount of Man sugars relative to the EOF broth obtained from the parent cell fermented under the same conditions.

14. The method of claim 13, wherein the endogenous gene encodes a functional alpha-mannosidase protein comprising at least about 50% to 100% identity to an amino acid sequence selected from the group consisting of SEQ ID NO: 2, SEQ ID NO: 4, SEQ ID NO: 21, SEQ ID NO: 23, SEQ ID NO: 27 and SEQ ID NO: 29.

15. (canceled)

16. The method of embodiment 4, wherein the parental cell is a Trichoderma sp. cell comprising a mutated glucosidase IIα (gls2a^{Stop}) allele encoding a truncated glucosidase II α-subunit (GIIα^{Stop}) protein, wherein the modified a T. reesei cell comprises a restored glucosidase IIα (gls2a^{R}) allele encoding a native glucosidase II α-subunit (GIIα) protein.

17-20. (canceled)


SEQUENCE LISTING (36 sequences)

SEQ ID NO: 1 - DNA, 1572 bp, Trichoderma reesei
  source 1..1572: mol_type = other DNA
atgagattccctagcagctccgtccttgccctcgggctcatcggacctgcgctggcgtatccaaagccgggcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 2 - protein, 523 aa, Trichoderma reesei
MRFPSSSVLALGLIGPALAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLA

SEQ ID NO: 3 - DNA, 2523 bp, Trichoderma reesei
  source 1..2523: mol_type = other DNA
atgcggacgagagctgatagaaatcctcgtcaccctctccctcgccgtcatcctctccggccaatcgcccgcaaacgcagccgcgacagcacaacagccgtccaacggcctctcgtacatcaacccgctcatcggcaccaccaacggcggcaacgtcttcgccggcgcaacgctcccctacggcctcgcaaaggcgtcggccgacgtcgacggccagaacacgggcggcttcgggctcgacggcagcaacgtcgtcggcttctcgagcgtccacgacagcggcacggggggcaaccccagcctgggcaacttcccgctcttcccgcagctctgcccggacgatggcgacatcaactcgtgccggttccgcatcggcgacaggaagctgcactatgcgaacgactcggtgactgcgcggccgggatactttgggctgaggctggagtcgggcgtcgcggcgaacatgacggtctcgcagcacgcggcgctgtatcggttcacgtttccgcaggactcggacggggacaagcatccgctgattcttctggacctgacggatctgtggcagagccggcagaacgcctcgatccaggtcgacgagaggacgggccgcatgacgggcaacggcacgtttctgcccagcttcggcgccggatcgtacgtgatgcatttctgcgccgacttcttcggtccggacatccacgagacgggcgtctgggtcaacagccgcgcgggcaccgaccccaagcatgtcttccttacacggggcttcaacctgttctaccttgagggcggcggcttcgtgcgcgtcaagccccccggcgaggacggcgtcgtgacggtgcgcatgggcatcagctacatcagcagcgagcaggcgtgtcgcagcgccgagcgcgagattcccaatccgctcaaggactttgatcggctcgttggcgaggcgcagagcgcgtggacggagaagctgagtccgatatccgtcaagagcggcggtgcaacagaggacttgatgacgagcttctggagcggagtctatcgcaacatgatatcgccgcagaactacacgggcgagaatccgctatggaagagcgacaagccgtatttcgactctttctactggtgagttgtgctcaagtctaaatctaaagggctcgaggggagatctgactgtgtgtgtgtgtgtgtgtgtgtgcagcatctgggacagcttccgtgtccagcatcctctgctgaccattctcgatccccatgctcagacgcagatggtggaggccctgctggacatttacaagcatgagggctggatgcccgactgccgcatgtccctctgcaagggctggacccaaggaggctccaatgccgacgttgtcatcgcggacgcctttgcgaagaacctcagcacgacaatcgactgggagcttgccctggaagccgttatggccgacgccgaaaacgagccgcaggagtggtcgtatcacggccgaggcggcctccacagctggaagaagctcgattacatcccctacctcgactttgacccgtacggcttcgggaccaactcgcgcagcatctcgcggacgcttgaatatgcgtacgacgactactgtctctcggagctggctggcgggctggggtggagggacttgcaggcaaagtaccagaggcgaagcatgaactggaaaaacctgtggaaggcggaccagacgtcgttgatcaacggcaccgacacggggttcaggggcttcttccagccgcggtaccagaacgggacttgggggttccaggacccgattgcgtgttctgcgctggctggattctgctcgctgactacgaatccgagcgagacgtttgaagccagcatctggcagtatctgttgtaagtcgaagacgcccactgccgagttgtcacgaccaagctgacttaccttaacgtacagctacgtccctcactccgtctcgtccctcatttcgctcctcggcggcgacgacgccatgatttcgcgcctcgacttcttccacacctcgggcctggcggacatctccaacgagcccgtcttcttcaccgtcttcctctaccactacacggggcggccgggcctctcgacgaagcgcatccaccagtacgtccccgccgacttcaactcgtcgcccggcggcctcccgggcaacgacgactcgggcgccatgggcgcgttcctcgtcttctccgtcatgggcctgttccccgtggcgggacagaacgtgtatctgatctcgccgccgtttgtggaggagattagcatccggcatccggtgacggggaagacggcgacggtgcggaatatcgggttcgatgcgtcgtatgagaagatttatgtgcagagcgcgagggtgaatgggaggccgtggacgaggtcgtggattgggcacgagtttttcaccgaggggtggacgctggagttggtgcttggggcggaggagtcggggtgggggagggatgtcaaggataggccgccgagctggtctggagatgtgatgtga

SEQ ID NO: 4 - protein, 794 aa, Trichoderma reesei
MRTRADRILVTLSLAVILSGQSPANAAATAQQPSNGLSYINPLIGTTNGGNVFAGATLPYGLAKASADVDGQNTGGFGLDGSNVVGFSSVHDSGTGGNPSLGNFPLFPQLCPDDGDINSCRFRIGDRKLHYANDSVTARPGYFGLRLESGVAANMTVSQHAALYRFTFPQDSDGDKHPLILLDLTDLWQSRQNASIQVDERTGRMTGNGTFLPSFGAGSYVMHFCADFFGPDIHETGVWVNSRAGTDPKHVFLTRGFNLFYLEGGGFVRVKPPGEDGVVTVRMGISYISSEQACRSAEREIPNPLKDFDRLVGEAQSAWTEKLSPISVKSGGATEDLMTSFWSGVYRNMISPQNYTGENPLWKSDKPYFDSFYCIWDSFRVQHPLLTILDPHAQTQMVEALLDIYKHEGWMPDCRMSLCKGWTQGGSNADVVIADAFAKNLSTTIDWELALEAVMADAENEPQEWSYHGRGGLHSWKKLDYIPYLDFDPYGFGTNSRSISRTLEYAYDDYCLSELAGGLGWRDLQAKYQRRSMNWKNLWKADQTSLINGTDTGFRGFFQPRYQNGTWGFQDPIACSALAGFCSLTTNPSETFEASIWQYLFYVPHSVSSLISLLGGDDAMISRLDFFHTSGLADISNEPVFFTVFLYHYTGRPGLSTKRIHQYVPADFNSSPGGLPGNDDSGAMGAFLVFSVMGLFPVAGQNVYLISPPFVEEISIRHPVTGKTATVRNIGFDASYEKIYVQSARVNGRPWTRSWIGHEFFTEGWTLELVLGAEESGWGRDVKDRPPSWSGDVM

SEQ ID NO: 5 - DNA, 2955 bp, Trichoderma reesei
  source 1..2955: mol_type = other DNA
atgaggtcgacgatggggctgtcctggaagtggacggcactcttcagccttttaggtgccattctgtgcctgatcgggcctgcctgtatgcatcatgaacacatcgcggctttggaagcatcttgctgacattgaaaccttctagtggccgtcaaggaacacgagttcaaaaagtgccaccaggccggcttctgcaaccgaaaccgtgcattggccgaccttgcggcttcccagagctcgacctgggtgtctccctacaaggctgtcttcgaatctccctcgttggaagacggaaagattcagggtgttatcctcaaaacgatcaatgccgccggcgacactgtccgactgccagtcacaatctcattcctcgaatcgggtgttgctcgcctcaccattgacgaagagaggaggcaaaacaaggacattgagctgaggcacggcagcgccgcaagaaaggaacgctacaacgaggctgcgaactggtccgttgttggcggccttgagccggctttgaaagccgagattgtccaccaggacgactctcaaatcaatgtcaaatacggtcccgaaggcaactttgaggccgtcatcagactctcgccctttaggattgatttccggagagacggtgtctcgcacatcgagctcaacgaacgcggcctgctcaatgttgagcattggagaccaaaggtcgagagaccagagggagaagaaaacaccgaggaggacgaaagcacttggtgggacgaaacgtttaacggacataccgataccaagccacgtggccccgaaagtgtggccttggacatctcgttccatggatacgagcacgtctatggtattcccgagcatacgggacctctttctctcaaggagactcgaggcggcgaaggcaactatgcagaaccctatcgcatgtacaacgccgacgtcttcgagtatatccttgacagccccatgaccctctacggctccatccctttcatgcaggctcaccgaaaggattcgtccgtcggtctcttgtggctcaatgccgccgatacgtgggttgatatcaccaaggtcaagggcagtactaaccctctgagcctgacctccggtgccccaaagaacacccagacccactggatctccgagagcggtatcatcgatctctttgtcttcttgggccccacgccccaggacattacgaagaagtatggcgagctgacgggaacaaccgctatgcctcaagagtttgccctcggctaccaccagtgccgatggaattacttctccgaagacgacgtcaaggacgttgatcggaggtttgacaaggctcatatcccgtacgacgtcatctggctggatatcgaatacaccgatgagatcaagtacttcacctgggatcctcactcattcaccgaccccatcaccatgggtaaacagttggacagccacggcagaaaactggtgacaatcattgatcctcacatcaagagggttgataactatcctatcaatgagcaactcctggctcttgacctcgccattcatgacaaggacggcaaggcatacgaaggctcgtgctggccaggcaattccaactggatcgactgcttcaaccccaaggcccgtgagtggtggaagggtctgtacaaatacgatcagttcaagggaacaatggagaacacctttatctggaacgacatgaacgaaccttccgtctttgaagggccagagactaccatgcccaaggacaatctccactgggacaactgggagcaccgagatgtccacaacctgaacggaatgacgtatcatcactctacctttgaagctctgaagagccgcaagaagggcgagtaccgacgtccgtttgtgctgacccgagcgttcttctccggatcccagcgcttcggtgctatgtggaccggtgacaacctggcggactggggccacctgcagacttccgtcaccatgctcatcaaccagggtatctctggtttcccgttctcgggcgctgacgtcgcagggttctttggagaccctgagaaggacctgcttgcccgttggtaccagacggctgcattctatccattcttccgcgcccatgctcacattgacacccgtcgccgcgaaccatacctgcttggcgagccttacactgccattgttactgcagcgctgcggctgcgatactctctcttgcccgcttggtacactgccttcttccacgcgaaccgagatggtagccctatcctccgccccatgttttggacgcatccgtctgccgagggaggcttggcgattgatgaccagttcttcctggcatctactggcttgctcgttaagcccgttgcagagaaagacaagtacagcgctgacatctggattccggatgacgaggtctactacgaatacgacacgtacaacgtggcgaagaccgagcagggcaagcacgtcacatttgacgcccccattgaccgaatccctatcctgatgcgaggaggccacatcattcctcgccgcgatattccccgccgctcgagctctctcatgcgtttcgacccctatacccttgtcgtctccgtctccaaggatggccaggccgaaggagagctgtacgtggacgatggcgatagctacgagtaccaggatggtcaatacattcatcgccagttcagcctaaaggacgatgtcctttcttcagtcgatgctgaaggccgagacactcgaaagatcaagcccggcaagtggctcaaggctatgcagaatgtccacatcgacaagatcatcatcgttggcgctcctgcttcatgggaccgagaagcggtgcagatcgaatcagacggacgcgcctgggctgcccaggtggtctaccacgccgccgacaaggacagggctgcatttgcaactgtgacccatgtgaaggcccgtgttggggatgactggagcatcaagctggcttag

SEQ ID NO: 6 - protein, 964 aa, Trichoderma reesei
MRSTMGLSWKWTALFSLLGAILCLIGPALAVKEHEFKKCHQAGFCNRNRALADLAASQSSTWVSPYKAVFESPSLEDGKIQGVILKTINAAGDTVRLPVTISFLESGVARLTIDEERRQNKDIELRHGSAARKERYNEAANWSVVGGLEPALKAEIVHQDDSQINVKYGPEGNFEAVIRLSPFRIDFRRDGVSHIELNERGLLNVEHWRPKVERPEGEENTEEDESTWWDETFNGHTDTKPRGPESVALDISFHGYEHVYGIPEHTGPLSLKETRGGEGNYAEPYRMYNADVFEYILDSPMTLYGSIPFMQAHRKDSSVGLLWLNAADTWVDITKVKGSTNPLSLTSGAPKNTQTHWISESGIIDLFVFLGPTPQDITKKYGELTGTTAMPQEFALGYHQCRWNYFSEDDVKDVDRRFDKAHIPYDVIWLDIEYTDEIKYFTWDPHSFTDPITMGKQLDSHGRKLVTIIDPHIKRVDNYPINEQLLALDLAIHDKDGKAYEGSCWPGNSNWIDCFNPKAREWWKGLYKYDQFKGTMENTFIWNDMNEPSVFEGPETTMPKDNLHWDNWEHRDVHNLNGMTYHHSTFEALKSRKKGEYRRPFVLTRAFFSGSQRFGAMWTGDNLADWGHLQTSVTMLINQGISGFPFSGADVAGFFGDPEKDLLARWYQTAAFYPFFRAHAHIDTRRREPYLLGEPYTAIVTAALRLRYSLLPAWYTAFFHANRDGSPILRPMFWTHPSAEGGLAIDDQFFLASTGLLVKPVAEKDKYSADIWIPDDEVYYEYDTYNVAKTEQGKHVTFDAPIDRIPILMRGGHIIPRRDIPRRSSSLMRFDPYTLVVSVSKDGQAEGELYVDDGDSYEYQDGQYIHRQFSLKDDVLSSVDAEGRDTRKIKPGKWLKAMQNVHIDKIIIVGAPASWDREAVQIESDGRAWAAQVVYHAADKDRAAFATVTHVKARVGDDWSIKLA

SEQ ID NO: 7 - DNA, 2954 bp, Trichoderma reesei
  source 1..2954: mol_type = other DNA
atgaggtcgacgatggggctgtcctggaagtggacggcactcttcagccttttaggtgccattctgtgcctgatcgggcctgcctgtatgcatcatgaacacatcgcggctttggaagcatcttgctgacattgaaaccttctagtggccgtcaaggaacacgagttcaaaaagtgccaccaggccggcttctgcaaccgaaaccgtgcattggccgaccttgcggcttcccagagctcgacctgggtgtctccctacaaggctgtcttcgaatctccctcgttggaagacggaaagattcagggtgttatcctcaaaacgatcaatgccgccggcgacactgtccgactgccagtcacaatctcattcctcgaatcgggtgttgctcgcctcaccattgacgaagagaggaggcaaaacaaggacattgagctgaggcacggcagcgccgcaagaaaggaacgctacaacgaggctgcgaactggtccgttgttggcggccttgagccggctttgaaagccgagattgtccaccaggacgactctcaaatcaatgtcaaatacggtcccgaaggcaactttgaggccgtcatcagactctcgccctttaggattgatttccggagagacggtgtctcgcacatcgagctcaacgaacgcggcctgctcaatgttgagcattggagaccaaaggtcgagagaccagagggagaagaaaacaccgaggaggacgaaagcacttggtgggacgaaacgtttaacggacataccgataccaagccacgtggccccgaaagtgtggccttggacatctcgttccatggatacgagcacgtctatggtattcccgagcatacgggacctctttctctcaaggagactcgaggcggcgaaggcaactatgcagaaccctatcgcatgtacaacgccgacgtcttcgagtatatccttgacagccccatgaccctctacggctccatccctttcatgcaggctcaccgaaaggattcgtccgtcggtctcttgtggctcaatgccgccgatacgtgggttgatatcaccaaggtcaagggcagtactaaccctctgagcctgacctccggtgccccaaagaacacccagacccactggatctccgagagcggtatcatcgatctctttgtcttcttgggccccacgccccaggacattacgaagaagtatggcgagctgacgggaacaaccgctatgcctcaagagtttgccctcggctaccaccagtgccgatggaattacttctccgaagacgacgtcaaggacgttgatcggaggtttgacaaggctcatatcccgtacgacgtcatctggctggatatcgaatacaccgatgagatcaagtacttcacctgggatcctcactcattcaccgaccccatcaccatgggtaaacagttggacagccacggcagaaaactggtgacaatcattgatcctcacatcaagagggttgataactatcctatcaatgagcaactcctggctcttgacctcgccattcatgacaaggacggcaaggcatacgaaggctcgtgctggccaggcaattccaactggatcgactgcttcaaccccaaggcccgtgagtggtggaagggtctgtacaaatacgatcagttcaagggaacaatggagaacacctttatctggaacgacatgaacgaaccttccgtctttgaagggccagagactaccatgcccaaggacaatctccactgggacaactgggagcaccgagatgtccacaacctgaacggaatgacgtatcatcactctacctttgaagctctgaagagccgcaagaagggcgagtaccgacgtccgtttgtgctgacccgagcgttcttctccggatcccagcgcttcggtgctatgtggaccggtgacaacctggcggactggggccacctgcagacttccgtcaccatgctcatcaaccagggtatctctggtttcccgttctcgggcgctgacgtcgcagggttcttggagaccctgagaaggacctgcttgcccgttggtaccagacggctgcattctatccattcttccgcgcccatgctcacattgacacccgtcgccgcgaaccatacctgcttggcgagccttacactgccattgttactgcagcgctgcggctgcgatactctctcttgcccgcttggtacactgccttcttccacgcgaaccgagatggtagccctatcctccgccccatgttttggacgcatccgtctgccgagggaggcttggcgattgatgaccagttcttcctggcatctactggcttgctcgttaagcccgttgcagagaaagacaagtacagcgctgacatctggattccggatgacgaggtctactacgaatacgacacgtacaacgtggcgaagaccgagcagggcaagcacgtcacatttgacgcccccattgaccgaatccctatcctgatgcgaggaggccacatcattcctcgccgcgatattccccgccgctcgagctctctcatgcgtttcgacccctatacccttgtcgtctccgtctccaaggatggccaggccgaaggagagctgtacgtggacgatggcgatagctacgagtaccaggatggtcaatacattcatcgccagttcagcctaaaggacgatgtcctttcttcagtcgatgctgaaggccgagacactcgaaagatcaagcccggcaagtggctcaaggctatgcagaatgtccacatcgacaagatcatcatcgttggcgctcctgcttcatgggaccgagaagcggtgcagatcgaatcagacggacgcgcctgggctgcccaggtggtctaccacgccgccgacaaggacagggctgcatttgcaactgtgacccatgtgaaggcccgtgttggggatgactggagcatcaagctggcttag

SEQ ID NO: 8 - protein, 807 aa, Trichoderma reesei
MRSTMGLSWKWTALFSLLGAILCLIGPALAVKEHEFKKCHQAGFCNRNRALADLAASQSSTWVSPYKAVFESPSLEDGKIQGVILKTINAAGDTVRLPVTISFLESGVARLTIDEERRQNKDIELRHGSAARKERYNEAANWSVVGGLEPALKAEIVHQDDSQINVKYGPEGNFEAVIRLSPFRIDFRRDGVSHIELNERGLLNVEHWRPKVERPEGEENTEEDESTWWDETFNGHTDTKPRGPESVALDISFHGYEHVYGIPEHTGPLSLKETRGGEGNYAEPYRMYNADVFEYILDSPMTLYGSIPFMQAHRKDSSVGLLWLNAADTWVDITKVKGSTNPLSLTSGAPKNTQTHWISESGIIDLFVFLGPTPQDITKKYGELTGTTAMPQEFALGYHQCRWNYFSEDDVKDVDRRFDKAHIPYDVIWLDIEYTDEIKYFTWDPHSFTDPITMGKQLDSHGRKLVTIIDPHIKRVDNYPINEQLLALDLAIHDKDGKAYEGSCWPGNSNWIDCFNPKAREWWKGLYKYDQFKGTMENTFIWNDMNEPSVFEGPETTMPKDNLHWDNWEHRDVHNLNGMTYHHSTFEALKSRKKGEYRRPFVLTRAFFSGSQRFGAMWTGDNLADWGHLQTSVTMLINQGISGFPFSGADVAGFLETLRRTCLPVGTRRLHSIHSSAPMLTLTPVAANHTCLASLTLPLLLQRCGCDTLSCPLGTLPSSTRTEMVALSSAPCFGRIRLPREAWRLMTSSSWHLLACSLSPLQRKTSTALTSGFRMTRSTTNTTRTTWRRPSRASTSHLTPPLTESLS

SEQ ID NO: 9 - RNA, 20 nt, synthetic construct
  source 1..20: mol_type = other RNA
ggttcttggagaccctgaga

SEQ ID NO: 10 - DNA, 90 bp, synthetic construct
  source 1..90: mol_type = other DNA
gtttcccgttctcgggcgctgacgtcgcagggttctttggagaccctgagaaagacctgcttgcccgttggtaccagacggctgcattct

SEQ ID NO: 11 - RNA, 20 nt, synthetic construct
  source 1..20: mol_type = other RNA
ctaatctcctccacaaacgg

SEQ ID NO: 12 - RNA, 20 nt, synthetic construct
  source 1..20: mol_type = other RNA
gctctcgtccgcatcctgac

SEQ ID NO: 13 - DNA, 200 bp, synthetic construct
  source 1..200: mol_type = other DNA
cataaatcttctcatacgacgcatcgaacccgatattccgcaccgtcgccgtcttccccgtcaccggatgccggatgctaatctcctccacaaactaagtagttagcgggaaggagaggctgattgaagcgacgatggcctcaatcaactctcgcgaacatataccatgtatgtatatatatatatatattcaccaataa

SEQ ID NO: 14 - DNA, 200 bp, synthetic construct
  source 1..200: mol_type = other DNA
ttattggtgaatatatatatatatatacatacatggtatatgttcgcgagagttgattgaggccatcgtcgcttcaatcagcctctccttcccgctaactacttagtttgtggaggagattagcatccggcatccggtgacggggaagacggcgacggtgcggaatatcgggttcgatgcgtcgtatgagaagatttatg

SEQ ID NO: 15 - RNA, 20 nt, synthetic construct
  source 1..20: mol_type = other RNA
gcaacagctttgatgatgag

SEQ ID NO: 16 - RNA, 20 nt, synthetic construct
  source 1..20: mol_type = other RNA
ggtaagctgcgagctcgtga

SEQ ID NO: 17 - DNA, 200 bp, synthetic construct
  source 1..200: mol_type = other DNA
cagtcaaggccgcattccagacgtcgtggaacgcttaccaccattttgcctttccccatgacgacctccacccggtcagcaacagctttgatgataagtgagtagggacggaacctcagtcctttcataaagtggcgaatttgtatcttactttggcctagaaacggctggggctcgtcggcaatcgatggcttggacac

SEQ ID NO: 18 - DNA, 2992 bp, Aspergillus niger
  source 1..2992: mol_type = other DNA
atgtccaaccgttggaccctactgctgtccttggtgatcctactcggatgccttgtcatccccggaggtaagcctatccaatcacactcctagtggtgaggctctccttgacatctaacaatgacgcatagtcaccgtgaaacacgagaacttcaagacatgttctcaatcgggcttctgtaagcggaacagagcattcgccgacgatgctgccgcccaaggttcctcctgggcctccccatacgaactcgactcatcctccatccagttcaaggatggccaattgcacggaaccattctcaagtccgtctcccccaacgagaaagtcaagctgcctctcgttgtctccttcctcgagtccggcgccgcccgagttgttgtcgatgaggaaaagcgcatgaacggtgacatccagcttcgacacgatagcaaagcacgcaaggaacgctacaatgaggcagagaaatgggtgttggttggtggcctggagttgagcaaaaccgcgaccttgagacctgaaaccgagtctggctttaccagagtcttgtacggtccggacaaccagttcgaggctgtcatccgccacgccccctttagcgccgacttcaagagggatggccaaacccacgttcaattgaacaacaagggctaccttaacatggagcattggcgccctaaggtagaggtcgaaggcgagggcgagcagcaaacccaggaagatgaaagcacttggtgggatgagagctttggtggaaacacggacaccaagcccaggggtcccgagagtgtgggattggatatcaccttccctggctacaagcatgtttttggaattcctgagcatgctgactctctctccttaaaggaaactcggtaagctagtcgcgcagtgacattttccatctcgcagaactgacagcgtcccagaggtggtgaagggaatcacgaagagccctaccgcatgtacaatgcggatgtatttgagtacgagctgagcagtcccatgaccttgtatggtgctattccattcatgcaggcacatcgcaaggactccaccgtcggtgtcttctggctgaatgctgcagagacctgggtggacattgtcaagtctacctcatctcctaaccctcttgctctcggcgtgggcgccaccactgacacccagagtcattggttttcggagtccggccagctcgacgtgttcgttttccttggtcctaccccacaggaaatcagcaagacctatggtgaactcaccggctacactcagttgcctcaacattttgccattgcttatcaccagtgccgctggaactacatcactgatgaggatgtcaaggaggtcgatcgcaactttgacaagtaccagatcccctacgatgtcatctggctggacatcgaatataccgatgacagaaagtatttcacctgggatccactcagtttccctgatccgatcagcatggaggagcagctcgatgagtcggagcgcaaactcgtcgttatcattgacccgcacatcaagaaccaggacaagtacagcatcgtccaagaaatgaagagcaaagacttggccactaagaacaaggacggtgagatctacgacgggtggtgttggcctggctcttctcactggatcgataccttcaaccccgccgccatcaaatggtgggtcagcttattcaagtttgacaagttcaaggggacgctgtccaatgtcttcatttggaacgacatgaacgagccctcggttttcaacggtcccgaaaccacgatgcccaaggataaccttcatcatggcaactgggagcaccgtgacatccataacgttcatggaatcaccctggtcaatgccacctacgatgcccttctagagcgcaagaagggcgagatccgtcggcctttcattctgacacggtcatattatgctggtgctcaacggatgtctgctatgtggacgggtgataaccaggctacttgggaacacttggccgcttccatccctatggttctgaacaacggcattgcgggcttcccctttgccggtgctgacgtgggcggtttcttccagaaccctagcaaggagctcttgaccagatggtaccaagctggtatttggtaccccttcttccgggcccacgcgcatattgacacgcgccggagagagccgtatctgattgccgagccacaccggtctatcatctcccaggctatccgcctgaggtatcagcttctccccgcctggtacactgccttccacgaagcttccgtgaacggaatgccgatcgtgaggccgcagtactacgctcacccttgggatgaggctggctttgccattgacgaccagctttatctcggctccaccggtcttcttgctaagcctgttgtctccgaggaggccaccacggccgacatttaccttgctgacgacgaaaagtactatgactactttgactacaccgtctaccagggagccggaaagcggcatacggtgcctgctcctatggagactgtgccattgctgatgcagggtggccatgtaatcccccgcaaggaccgtcctcgccgcagtagcgccttgatgagatgggatccgtacactcttgttgtggtcttggataagaacggtcaagccgatggctctctctacgtggatgacggtgagacgttcgactataagcgtggagcttatatccaccgccgtttccgcttccaggagtctgccctggtctcggaggatgttggcaccaagggtcctaagacggccgagtacttgaagaccatggccaacgttcgtgttgagcgggtggtggtagttgatcctcctaaggaatggcagggtaagaccagtgtgactgtcattgaggatggagcttcggcggcttcgacagcctctatgcagtaccacagccagcccgatggcaaggccgcatatgcggtggtgaagaaccccaatgtcggcattggaaagacatggcggattgagttttag

SEQ ID NO: 19 - protein, 957 aa, Aspergillus niger
MSNRWTLLLSLVILLGCLVIPGVTVKHENFKTCSQSGFCKRNRAFADDAAAQGSSWASPYELDSSSIQFKDGQLHGTILKSVSPNEKVKLPLVVSFLESGAARVVVDEEKRMNGDIQLRHDSKARKERYNEAEKWVLVGGLELSKTATLRPETESGFTRVLYGPDNQFEAVIRHAPFSADFKRDGQTHVQLNNKGYLNMEHWRPKVEVEGEGEQQTQEDESTWWDESFGGNTDTKPRGPESVGLDITFPGYKHVFGIPEHADSLSLKETRGGEGNHEEPYRMYNADVFEYELSSPMTLYGAIPFMQAHRKDSTVGVFWLNAAETWVDIVKSTSSPNPLALGVGATTDTQSHWFSESGQLDVFVFLGPTPQEISKTYGELTGYTQLPQHFAIAYHQCRWNYITDEDVKEVDRNFDKYQIPYDVIWLDIEYTDDRKYFTWDPLSFPDPISMEEQLDESERKLVVIIDPHIKNQDKYSIVQEMKSKDLATKNKDGEIYDGWCWPGSSHWIDTFNPAAIKWWVSLFKFDKFKGTLSNVFIWNDMNEPSVFNGPETTMPKDNLHHGNWEHRDIHNVHGITLVNATYDALLERKKGEIRRPFILTRSYYAGAQRMSAMWTGDNQATWEHLAASIPMVLNNGIAGFPFAGADVGGFFQNPSKELLTRWYQAGIWYPFFRAHAHIDTRRREPYLIAEPHRSIISQAIRLRYQLLPAWYTAFHEASVNGMPIVRPQYYAHPWDEAGFAIDDQLYLGSTGLLAKPVVSEEATTADIYLADDEKYYDYFDYTVYQGAGKRHTVPAPMETVPLLMQGGHVIPRKDRPRRSSALMRWDPYTLVVVLDKNGQADGSLYVDDGETFDYKRGAYIHRRFRFQESALVSEDVGTKGPKTAEYLKTMANVRVERVVVVDPPKEWQGKTSVTVIEDGASAASTASMQYHSQPDGKAAYAVVKNPNVGIGKTWRIEF

SEQ ID NO: 20 - DNA, 1787 bp, Aspergillus niger
  source 1..1787: mol_type = other DNA
cttatttatacccgattccgtccgctccttgacgtgctctcattcttgcgtcctgtttccgtcgtttgtgcaagacgagcttggtttttctattcgggtttgctcactcggttgtcactcatttcgccaaaccatgcatctcagctcgctttccttgtccctgacggccttggccatcgtcagtccttctgctgcttatcctcacttgggttcttcccagcctgtgcttcacactaattccgacaccactcaatcgcgcgcagacgccatcaaggctgccttctcgcacgcctgggatggctacctccaatacgccttccctcatgacgagctgcaccccgtctcgaatggctatggcgactcccggaatggatggggcgcctctgccgtggatgcactctccacagcagtgattatgcgcaatgctaccattgtcaatcagatcctggaccacgtggccaagattgactactccaagacaaacaccaccgtgagcctcttcgagaccaccatccgctacctgggtggcatgctgtccggatatgacctgctgaaggggcccgtgtccgacctggtgcaagactcgtcgaaaatcgacgtgctcttgacccaatcgaagaacctcggcgacgttctcaaattcgccttcgatacaccctcgggggttccatacaacaatctcaacattacctcaggggggaatgatggagccaagaccaatggtcttgctgtgacgggtacactggcgctggaatggacgcgcctgtcggaccttacgggtgacaccacgtatgccgacctgagccagaaggccgagtcttacctcctcaacccgcagcctaagtctgcggagccattcccgggcttggtaggaagcaacatcaacatcagtaatgggcagttcacggatgcgcaggtgagctggaatggcggcgatgactcttactacgagtacctgatcaaaatgtacgtatacgaccccaagcggttcggtctctataaagaccgctgggtggccgcggcgcaatcgaccatgcagcatctggcgtcgcacccctcgacccgcccggacctgaccttcctggcctcctacaacaacggaactttggggttgagctcgcagcacctgacttgcttcgacggaggtagcttcctgttgggaggcaccgtgctgaaccggacggatttcattgatttcggcctggacctagtgagcgggtgtcatgatacatacaactcgacgttgacgggaattggcccggagtcgttcagctgggataccagcgacatcccctcgagccagcagtcgctctacgagaaagccggattctacattactagtggcgcgtacattttgcgcccggaagtgattgagagtttctactatgcctggagagttaccgggcaggaaacggtaagttcgacttctccccctttggctcaatagtaactaacaaccatatcagtaccgggaatggatctggagcgcattcagcgcagtcaacgactactgccgcaccgactctggattttcaggtctgactgacgtgaacgcggccaacggcggcagccggtacgacaaccaggagagtttcctgtttgccgaggtgatgaagtattcgtacatggcgttcgcggaagatgcagcctggcaggtgcagcccggcagcgggaaccaatttgtgttcaacaccgaggcacacccagtgcgggtgagtagtacataaatggtagacattgttagagatggttttggtagcaatagaggacagctcaagtcacctaaa

SEQ ID NO: 21 - protein, 513 aa, Aspergillus niger
MHLSSLSLSLTALAIVSPSAAYPHLGSSQPVLHTNSDTTQSRADAIKAAFSHAWDGYLQYAFPHDELHPVSNGYGDSRNGWGASAVDALSTAVIMRNATIVNQILDHVAKIDYSKTNTTVSLFETTIRYLGGMLSGYDLLKGPVSDLVQDSSKIDVLLTQSKNLGDVLKFAFDTPSGVPYNNLNITSGGNDGAKTNGLAVTGTLALEWTRLSDLTGDTTYADLSQKAESYLLNPQPKSAEPFPGLVGSNINISNGQFTDAQVSWNGGDDSYYEYLIKMYVYDPKRFGLYKDRWVAAAQSTMQHLASHPSTRPDLTFLASYNNGTLGLSSQHLTCFDGGSFLLGGTVLNRTDFIDFGLDLVSGCHDTYNSTLTGIGPESFSWDTSDIPSSQQSLYEKAGFYITSGAYILRPEVIESFYYAWRVTGQETYREWIWSAFSAVNDYCRTDSGFSGLTDVNAANGGSRYDNQESFLFAEVMKYSYMAFAEDAAWQVQPGSGNQFVFNTEAHPVRVSST

SEQ ID NO: 22 - DNA, 2518 bp, Aspergillus niger
  source 1..2518: mol_type = other DNA
atgcgccccctcctccagccctaccttctcctccatatcctccctctcctcccgctcacccacagcaccgaccccaacaccgacatcctcacccacatcgaccccctcattggcaccaccaacggcggcaatgtcttcgccggtgcgaccctcccctacggcatggccaaagccgtcgccgacgtagacggccaaaacaccgccggcttctcctccgacaacagcaacatcaccggcttctccgccctgcacgactccggcaccggcgggaatccctccctcggcaacttccctttattccctcaatactgcgccgacgatctcctcgacaactgtccctttcccaagtccgcccgtgccatccactaccaaaatgactcggtcgttgcgcgacctgggtactttgccctaacgctgacgaatggtatacatgctgaaatgactactacgcagcatgcggcgctgtttcggttccagtttcctgatacagggcaggagaagttgagtccaatggtgttgttggatttgacggatttgtgggagagtaggcagaatgcgtcgattagtgtggatgagcacacggggcagatgaaggggaatgggacgtttctgccgagttttggggctggggagtatgtctcgtatgtatgtgcggattttgcgggggcgagtgtgagggataccggggtttgggtgaatgatgcgggtggagtggagaggaaggagttgtttgttacgagggggtttaataatttttatcttcaggcgggtgggttcgtgaggtttgagaggcctgacaatgggacggtgagtgtgagggttggggtgagttatattagtacggagaaggcgtgcgagaatgcaagaatggaaattcctagtccgttggaggactttgatgctatacgagcagctgcagaggatgcctggagggagaaattgagccctgttgcgatgaagccggggaatgcgagtgctgcgctgcagaccatgttctggacgggcacttataggactatgctggatccgcaggatttgacgggggagaatcccctctgggagagcgatgagccctactttgattctttctattggtaatacctacccacagcatctctctgcgaggcaacataactgatgtcttcagcatctgggacgccttccgggcccaacacccccttctcaccatcatcgacccccacactcaatcccgcatcatccgcagtctcctcgacacctatcgccacgaaggctggctcccggactgccgcatgagtctctgcaagggctggacccagggcggctccaatgcagacgtggtcctcgccgacgccttcgtcaagaacctcacaggcatcgactgggatctcgcctacgaggcaatggtcaacgacgccgagaacgagcccctggaatggtcctaccagggccgaggggggctccagagctggaagaacctgaactacatcccctaccacgactttgactacctaggtttcggcactaactcgcgcagcatctcccggaccctggaatacgcatacaacgacttttgcgtggcgactgttgcccgcggactaaacaaaacagacgattacttgaaatatctaactcgatcgacaaactggaagaatctatttaaacctgaccaacgctccttcttccccaatggctcggatacagggttcgtagggttcttccaacccaaatatcgcaatgggacatgggcacatcaggatcccatggattgtagtccgcttgccactggtgcaacatggtgctcgttgacgagtaatccctcggagacatttgagtcgagtatttgggaatatcagttgtattccccctaccccattaccctttacccattacccatacttctacttcatcactgaccaaagaactattgataaataacagttacgtcccccacgacctccccaccctaataacccactacctctccggaccccaagcattcacatcccgcctgaccaccttccactccacagctctagcagactacagcaacgagcctgtcttcctaaccgtctaccaataccactacgcctcgcgacccgccctctcctcgcgactcatccacagcttaatcccttcgcaattcaatgcctcccgctccgggatcccgggaaacgatgattcgggggcaatggcggcgttcacggtgtttggcctcatgggcctgtttccgaatcccggtcagaatgtgtatttgatttcggcgccgtttgtggaggaggtcagcgtgaagaatgcccttacggggagagaggcggtggtgagggtgaagaatgatgcgggggttggggtgggtgggaccgtggtgaggaaagcgtggttggatggggaggtgtatgcgaagagttggattgggcatgagttctttgtgaaggggggagtgttggaggttgagctgggggatgaagaggaggaaagtgagtgggggatgggggtgggagaggggccgccgagttgggggggagggatggagttgtag

SEQ ID NO: 23 - protein, 793 aa, Aspergillus niger
MRPLLQPYLLLHILPLLPLTHSTDPNTDILTHIDPLIGTTNGGNVFAGATLPYGMAKAVADVDGQNTAGFSSDNSNITGFSALHDSGTGGNPSLGNFPLFPQYCADDLLDNCPFPKSARAIHYQNDSVVARPGYFALTLTNGIHAEMTTTQHAALFRFQFPDTGQEKLSPMVLLDLTDLWESRQNASISVDEHTGQMKGNGTFLPSFGAGEYVSYVCADFAGASVRDTGVWVNDAGGVERKELFVTRGFNNFYLQAGGFVRFERPDNGTVSVRVGVSYISTEKACENARMEIPSPLEDFDAIRAAAEDAWREKLSPVAMKPGNASAALQTMFWTGTYRTMLDPQDLTGENPLWESDEPYFDSFYCIWDAFRAQHPLLTIIDPHTQSRIIRSLLDTYRHEGWLPDCRMSLCKGWTQGGSNADVVLADAFVKNLTGIDWDLAYEAMVNDAENEPLEWSYQGRGGLQSWKNLNYIPYHDFDYLGFGTNSRSISRTLEYAYNDFCVATVARGLNKTDDYLKYLTRSTNWKNLFKPDQRSFFPNGSDTGFVGFFQPKYRNGTWAHQDPMDCSPLATGATWCSLTSNPSETFESSIWEYQFYVPHDLPTLITHYLSGPQAFTSRLTTFHSTALADYSNEPVFLTVYQYHYASRPALSSRLIHSLIPSQFNASRSGIPGNDDSGAMAAFTVFGLMGLFPNPGQNVYLISAPFVEEVSVKNALTGREAVVRVKNDAGVGVGGTVVRKAWLDGEVYAKSWIGHEFFVKGGVLEVELGDEEEESEWGMGVGEGPPSWGGGMEL

SEQ ID NO: 24 - DNA, 3337 bp, Thermothelomyces thermophilus
  source 1..3337: mol_type = other DNA
ccattgatctcgtgtaaaccagacaccttgaaacaaacactggttcattgtcgaacaactattgagcacctcgacggccccggaggtctctcgatgaaggcgactgtgacataaccgccctttctcctttcttctttttagttttcctagatttggtcatcttgatcagcaatcgcgtcgtgctgtaacaagggtcccgaggacggccaacatgcacgtcccaggcccattgtcctcgcggtggacggcctaccttggcctgctgtccgcattttctgcagcatttgcacctgcaggtgagtcccgcgagcatctgatgccaccagacgaccgagttgacgatagtagtcgccgtcaaggagcatgacttcaaaaagtgccatcaatcgggcttttgtaagcgcaaccgcgaattcgccgaccacgccctcgccacgagctcctgggcgtcaccctacaatgtcgcaccagattccggatcgttcaaagatggccagtaccaggctgtgatcttgaagaccaccaacagcggcgagacggtttggcttcccattaccgtctctttccttgagtctggtacagcgcgagtcacggtggacgaagagaagcgccagaaaggagagattgagttgcgccatggcagcaaagcgaggaaagagcgctataacgaggcagagaaatgggccattgttggagggatgactttggacaaggaggccaaggtcgattatgaggacaagacgcagattaccgtcaagtacgggccaacctccaagttcgaggcaaccatcaaattctctcccttcagcatcgacttcaagcgggacggcatcagccacgtcaaactcaacgaccagggcctccttaacatcgagcactggaggccaaaggttgacaaaccggagcccgagaagaaggacggcgattcgactgaagagaataaggcagaggaagcaaaggaggagcccaagggagaggatgagtctacctggtgggaggagacatttggcggcaacaccgataccaagcctcgcggcccggagagcgtcggtcttgacatctccttcgtcggctacgaacacgtctacggcatcccctcccatgccagctcgctgtcgctgaagcagacccgtggcggcgagggcaactaccaggagccgtacaggatgtacaatgctgacgttttcgagtacatcctcgatagcccaatgactctctacggctctattcctttcatgcaggcccatcgtaaggactcgagcgtgggcgttttctggctgaatgccgcggagacgtgggttgacatcaccaaggcgaaagactccaagaacccgctctctctcggcggcaaggccaggaccaacacgcatacccactggttctccgagagcggtctgctggacgtttttgtcttcctcggcccgacgcccaaggatttgaccgccaggtacggtgagctcactggcaccactgcaatgccccaggagttcgctctcggataccatcagtgccgttggaactacgtctcggacgaggacgtaagggatgtggaccgcaagatggacaagttcaagatgccgtacgacgtcatttggctcgacatcgagtacactgacgagaagaagtactttacctgggacaaacactcgtttacagaccccatcggcatgggcaagcagctggacagtcacggcaggaagctggttaccatcattgatccgcacatcaagaataccgacaattaccccgtcgtggcagagctaaagtccaaggaactcggcgtaaagaacaaagatggcaacctcttcgagggctggtgctggccaggctcgtctcactggatcgacgccttcagtccggctgcgcgcgagtggtggtcgtcgcttttcaagtatgacaaattcaagggcaccatggaaaacacgtggatctggaacgacatgaacgagccgtcggtctttaacggtcccgaaaccacgatgcccaaggacaacctccacgacggaaactgggaacaccgcgacgtgcacaacctgaatggcctgacgttccacaacgcgacctaccatgcgctcctcacccggaagcccggcgagctccgccgcccgttcgtgctgacccggtccttctttgccggctcgcagcgggtcggcgccatgtggaccggcgacaaccaggctgcctgggaccacctcaaggcatcgattcccatggtcctcagccagggtatttcgggcttccccttctcgggcgccgacgtcggcggcttctttggcaacccggagaaggagcttttgacgcggtggtaccaagccggcgccttctaccccttcttccgggggcacgcgcacattgatgcgcgacgccgcgagccatacctcgccggcgagccgtacacgaccatcatcgccgcggcgcttcggctgcgctacagcctgctgccgtcgtggtacactgcgttccggcacgcccatctggacggtacgcccattatcaagcccatgttctacacgcatccgtccgaggaggccggcctcgctatcgaggaccagttcttcgtcggcaacacgggcctgctcgccaagcccgtcacggagaaggagaagacgacggtcgatgtgtggatccccgacggcgaggtctactatgactactttacgtaccaggtcatcccgacagtcaagggcaagacggtcacgctggatgcgccgatggagaagatcccgctgctgatgcgcggcgggcacgtgtttgcgcgccgcgacgtgccgcggcgctcgagcgcgctcatgcgctgggacgactacacgctcgtcgtgacggtgccgcgggaaaacaaggtcgccgagggcgacctgtacgtcgacgacggagactcgtttgagtaccagaacggccagtacatccaccgccggttcgtctacgacggcgcggccaagagcctcagctcggtggatgccgagggccgcgacgcggcctcgatccgggagggcgcgtggatgaagcagatgcgctccgtgggcgtgggcaagatcgtggtggtcggcgcgccggccagctgggcgggcaagaagagcgtcaaggtagagagcgagggcaaggtctgggaggccaggatggagtttacgcctgctgggcagggcagagccgcgttcgcggtgatcaagaaggtgggcgtgaggatcggggcggattggaaggtcgagttttgatttccaccactgggcttgtgcgtatttttgttggagtaggttctctctctcgtattattcgactttttgttttgggccccgttatgtcggtcatggtggagcctcgcaagcatcctgttttcgaa

SEQ ID NO: 25 - protein, 982 aa, Thermothelomyces thermophilus
MHVPGPLSSRWTAYLGLLSAFSAAFAPAVAVKEHDFKKCHQSGFCKRNREFADHALATSSWASPYNVAPDSGSFKDGQYQAVILKTTNSGETVWLPITVSFLESGTARVTVDEEKRQKGEIELRHGSKARKERYNEAEKWAIVGGMTLDKEAKVDYEDKTQITVKYGPTSKFEATIKFSPFSIDFKRDGISHVKLNDQGLLNIEHWRPKVDKPEPEKKDGDSTEENKAEEAKEEPKGEDESTWWEETFGGNTDTKPRGPESVGLDISFVGYEHVYGIPSHASSLSLKQTRGGEGNYQEPYRMYNADVFEYILDSPMTLYGSIPFMQAHRKDSSVGVFWLNAAETWVDITKAKDSKNPLSLGGKARTNTHTHWFSESGLLDVFVFLGPTPKDLTARYGELTGTTAMPQEFALGYHQCRWNYVSDEDVRDVDRKMDKFKMPYDVIWLDIEYTDEKKYFTWDKHSFTDPIGMGKQLDSHGRKLVTIIDPHIKNTDNYPVVAELKSKELGVKNKDGNLFEGWCWPGSSHWIDAFSPAAREWWSSLFKYDKFKGTMENTWIWNDMNEPSVFNGPETTMPKDNLHDGNWEHRDVHNLNGLTFHNATYHALLTRKPGELRRPFVLTRSFFAGSQRVGAMWTGDNQAAWDHLKASIPMVLSQGISGFPFSGADVGGFFGNPEKELLTRWYQAGAFYPFFRGHAHIDARRREPYLAGEPYTTIIAAALRLRYSLLPSWYTAFRHAHLDGTPIIKPMFYTHPSEEAGLAIEDQFFVGNTGLLAKPVTEKEKTTVDVWIPDGEVYYDYFTYQVIPTVKGKTVTLDAPMEKIPLLMRGGHVFARRDVPRRSSALMRWDDYTLVVTVPRENKVAEGDLYVDDGDSFEYQNGQYIHRRFVYDGAAKSLSSVDAEGRDAASIREGAWMKQMRSVGVGKIVVVGAPASWAGKKSVKVESEGKVWEARMEFTPAGQGRAAFAVIKKVGVRIGADWKVEF

SEQ ID NO: 26 - DNA, 2857 bp, Thermothelomyces thermophilus
  source 1..2857: mol_type = other DNA
actacggattaggggtgccaccttctcagtaaaagctgaaccttaaccccagaccgatttgatttgattgagctcctcgtttttctcttccttcgattatctatttaaatacgcgctcctggcatgcgcgtacatacgtgagcgcagggttggcacttgctatccgcggttttggtgaggtattgatcggttattggcctttttgtggcgcgggtcaacaagcgccatgcgtggtctttctgttgctggggcaagtgctgccgccagtccccttttgcttggcctagctgctgctgcacctcacaaacagcgcgctccgcagtatgtcgttaacaagcaacgcgccgacgaggttaaggaagctttccaggtgtcttgggatggttattacaaatacgctttccctcacgactcgttacggcccgtgtccaacacattcgcggatgacaggtaattaggcagggactttactttttttgttccttccattcgttcctcttactatttttcttttcttttcttttcttttttttttttttgcttctttctatttatttattcaaagctattcatttatttattgctgtgtttgaagaaggtgaagagggcggaggtttgataactgctaactagagacggccgatagaaatggatggggtgcgagcgctgtcgacgcgctcagcacggcgctggtcatggagaactgggatgtcgtgaaccagatattggagtacatcccaaccatcaacttcgacaacaccaccaccgaagtgtccctgttcgagaccacgattcgatatctcggtggactggtgtccggtagggctcgtccctctacataatatgcgcggtctctggctcatgtagtttatttcagcctacgacatcataaccaacagcctcagtctgggcatccaaaaacagccggacgtagaagccatcctgactcaggcgaagcggctggccgacaaccttaaagttgccttcgacacgcccagcggcgttccggacaacagcctctacttcaacccaccacggaagggcggatcaacgacgaacggcctcgccaccaccggcaccttgatccttgagtggacacgcctcagcgacctgaccggggactctgaatatgcggagctgacgcaaaaggcggagtcctacctgctgcaccccgagccggcgctgggcgagccatttcccggcctgctggggactaacattcgaatcgaggacgggaagtttgaagacggcaacggcagctggggcggcggaaccgacagcttctatgagtatctcatcaagatgtatatttacgaccccgagcggtttgccgagtaccgcgaccgctggatcgccgcggccgactcgtccattaagtacctcgtctcgcacccgaccacgaggccggagctgacgttcctcgctctgtggcggggcaaggagctgcggttctcatctcagcattgtgagcaacccaacccctacttacccacttccccccccttttttttttttttttttcccatactcatcagtttttcaaccgaagtggcctgcttccacggcggcaacttcatacttggcggcctcacgctcggcgaacaggcgtacacggatctcgggctcgcgctcgtcgaaggctgccacgcgacgtacgtgggcacggcgacgggaatcgggccggaagtcttcgcttggcaggactcgcagctgccgctcaacgcgagcaataaccagccgcctccagaggaccagaccgctttctacgagaagtcgggtttctggatcaccaacggccattacgtgctgcggcccgaggtcatcgaaagctactactacgcctaccgcgcaaccggcgacaccaagtaccaggactgggcctgggacgcattccagaggatcaacaagacatgccgcgtggggagcggattctcgagcatcaaggacgtgaatgctccagacgggggcggattcgacgacttccaggagagcttctggtttgccgaggtgctcaagtatagctatctgatccatgccgaggtgagttggctgacaatcggtgttctgggccgactctcagctaaaccacgatgggaacaggatgctccgtggcaggtcaaggccgataacacgaacgagtacgtgtggaacaccgaggcgcatccgatacgggtggctggagggaaataagagagaccagagtctcgggttcatgcctgggtggcatttttggatccaagaggtagttaggaatattatttgaaacattggtaataaggcaacattgaatttttctaggtaaggtataattaaacattgtctcccgttacttacgtctctcattcgacgacgtaatcagctgagcacctgctgggtccatgtcaggactcccaattactattcggtttgttaagattggactatgtgtaaaggtctctagatagtaattaatagtttatcagtgcctgatcaaatgtttcacttttagagtgacggcgtacttggccaagttatcctcctccattcaccatttcatttccgctgattacgtgtttccctttcacgcaccgccctcacgcttggcatactcgtcctctatggcttcctcgtagcgccggtctgcctccttctcggcttccgtcttgggcctggtgatgcccgcgccggtgttgactccggcgccggtcggcggtgcttgggtcctctccgagcctgaatgctgagtttgggtctgggtgcccaagtcgtggcctgtggaggaggcggtagaggtctggtgcggcattttttttgtccaaatggatgacagataaaggttatcgaaacggtgtcttgagtgttggtgttttggtgcgag

SEQ ID NO: 27 - protein, 521 aa, Thermothelomyces thermophilus
MRGLSVAGASAAASPLLLGLAAAAPHKQRAPQYVVNKQRADEVKEAFQVSWDGYYKYAFPHDSLRPVSNTFADDRNGWGASAVDALSTALVMENWDVVNQILEYIPTINFDNTTTEVSLFETTIRYLGGLVSAYDIITNSLSLGIQKQPDVEAILTQAKRLADNLKVAFDTPSGVPDNSLYFNPPRKGGSTTNGLATTGTLILEWTRLSDLTGDSEYAELTQKAESYLLHPEPALGEPFPGLLGTNIRIEDGKFEDGNGSWGGGTDSFYEYLIKMYIYDPERFAEYRDRWIAAADSSIKYLVSHPTTRPELTFLALWRGKELRFSSQHLACFHGGNFILGGLTLGEQAYTDLGLALVEGCHATYVGTATGIGPEVFAWQDSQLPLNASNNQPPPEDQTAFYEKSGFWITNGHYVLRPEVIESYYYAYRATGDTKYQDWAWDAFQRINKTCRVGSGFSSIKDVNAPDGGGFDDFQESFWFAEVLKYSYLIHAEDAPWQVKADNTNEYVWNTEAHPIRVAGGK

SEQ ID NO: 28 - DNA, 3313 bp, Thermothelomyces thermophilus
  source 1..3313: mol_type = other DNA
ctcaggggtccgagagatcggcatgctgttgcctacagactaaccacatgtgcctgtaattactgcagggcaacggaaacacacctccagatctgcaggtatggactgtacagtacatacatataggcatatagacatcctgtgcataggataatgaccgcccggctcccgacatcaacgtccggacgaacgctccccggcgaatgaccgtcggaagccaccactgtcagcaccgcggggactcgttaaccgcgatggctttacccgtactcctcatcgcatgctggtcggcgctggcgggtcaagtactggcccgcaacacctttgatccgctcgcgtacgttgatccgctgatcggagccagcaacggcggcaatgtgttcccgggcgcctcgctgccgtacggcatggcgaaagcagtcgccgataccgacagcggcagccggcagggcggcttcactatggatggcgccgccgtcaccggcttcagcaccatgcacgacagcgggaccggagggaatccgagcttgggcaactttgcactcttcgcctacacaagctgccccggtggcgacatcaaccgatgtgccttccccaagaagacgcgtgctgcctttggacgcttccgcaatagcaatgtctcagcaaagcccggcacctttgacatcaccttgaacagcggcatccgagccgagatgacgacgactcatcacgcctcgctcttcaggtttaccttcccgagcgacggggctgatgaggagccagcccgacccctcatcttgcaggatctcacagatctggccgattctaggcaggataacgctacagtcgccgtggaccctaagacgggccgcatcaccggcagcgcccgcttcctgcccagctttggcgccggcacgtttgtcttgcacttctgcaccgacttcaagggcgccgaggtagcggacagcggcgtattcgtcaatagtcgggccagcaccgaggtccacaacctgaccatctcaagatccatcaatggctatcccctccccgggggggcgtttgtgaggtttaactccggcgccgagcccatccttgtacggactgctaccagcttcatcagtgccgagcgagcgtgcgagcacgccgagaaggagattcccgacttcgacttcactgccgtctccaaggccgcaactgatgtctggagggccaagatgagtccgatcaaggtatcaacgaaacaggtgaacagctcgctgttgacaaacttctacagcggcatttaccggaccataatcaaccctcaaaactacacaggggagaatcccctgtggtccagcaaggagccgtactttgactcattctactggtcagacacacaactatcgcgcaggaggtggactcttgctgactgtggccagcatttgggaccaatttcgatcacagcttccgcttttgaccatcacggatcccacagccgtgactgggatggttcgctcgctgatcgacacataccgccatgtcggatggcttcccgattgccggatgagcctgagtaaaggctacactcaggtaaagcgaagctatcttttcgtgtgctggttttagacaaagacgcaagcaaaactgacaatgtcagggcgggtccaacgctgacgttgtgttagcggatgctcacgtcaagggactcagagagggcatcaacgtgagttcctctttctttcttttgtttttttcgattttttttccttcgacagattgctaaaaatcactgcggtgggacgatggatttgctgcggtggttaaagacgccgaggttgaaccttacgactggtgctgcgaagggcggggagggctcgacagctggaagtctctcggctacattccagtccaagacttcgactacaagggctttggtaccatgacgagaagcatctcgcgtactctggaatacgcctataacgattactgcatcgcccagatagccgcggctctcggcaagtctgccgagaaggaaaagtacctcgagtcgagcggcaactggcagaacctcttcaaggaagatcaaacctcgatctggtggaacggcaccgacacgggattcaccggcttttttcagccacgctacctcaacaagacctggggcttccaagatccgctaaactgcagcaatcttgacaccgccagcgtttgctcgctccagaacactggccgcgagacctttgagagcagcatctgggagtatggcttgtaggtcttcctcgcccttcccaaccgccctacccttccattcttactatcccgaaccggtagcactgacccggctcatcagcttcgtcccccacgaccaggcaaccctcataagcctctacggcggccccgccgccttcgtgtcccgcctcgactacctccacgacagcgggataacctacattggcaatgagccttccttcctcaccgtcttccaataccactatgccggccgtccggccctctcggcgcgccgagcccacttttacattcctgccttcttctcccctaccccgggcggcctgcccggaaacgacgacagcggcgcaatgggatccttcgtggccttttcgatgatgggcctgttccccaacccgggtcagaacgtctacctgatcacgccacccttcttcgagagcgtcaacatcacgcacccgctcacccacaggaccgcccgcatcaggaacgtcaactttgaccccacgtacaaggccatctacatccagagcgccacgcttgacggcaagctgtacaccaagaactgggtcgaccacagcttctttaccgagggcaaggagcttgtgctgacgctggggcggaacgagagcgcctggggcaccaaggtcgaggacctacccccgagtctgggagcgtatgaagggttcactaagaggagtgggtcactccggagaacggcagccccggatctctggagaaagacggcggctttcaaggggcagtttggaagcctcgaggagagtttgggcatgtgaacttctcacgggagagcggggaagtatacggctttagagctcgtgcgaaatcacatccgagagtggccatggccggcttcacacaagaaacagccgcaatccccggtgtttggctggttgaatgctgtggcagcgtctttcgaggtagcctccgattgccaagggaaacgatggtcgaaagcgtagaccgactcatttagctaaggtggcctagcatgatgctatcgaagcagccgccgcttgtttcaatagcaacgaacgatagaggctgtgtgggtatctaactttttaaatctcaacccaatacg

SEQ ID NO: 29 - protein, 825 aa, Thermothelomyces thermophilus
MALPVLLIACWSALAGQVLARNTFDPLAYVDPLIGASNGGNVFPGASLPYGMAKAVADTDSGSRQGGFTMDGAAVTGFSTMHDSGTGGNPSLGNFALFAYTSCPGGDINRCAFPKKTRAAFGRFRNSNVSAKPGTFDITLNSGIRAEMTTTHHASLFRFTFPSDGADEEPARPLILQDLTDLADSRQDNATVAVDPKTGRITGSARFLPSFGAGTFVLHFCTDFKGAEVADSGVFVNSRASTEVHNLTISRSINGYPLPGGAFVRFNSGAEPILVRTATSFISAERACEHAEKEIPDFDFTAVSKAATDVWRAKMSPIKVSTKQVNSSLLTNFYSGIYRTIINPQNYTGENPLWSSKEPYFDSFYCIWDQFRSQLPLLTITDPTAVTGMVRSLIDTYRHVGWLPDCRMSLSKGYTQGGSNADVVLADAHVKGLREGINWDDGFAAVVKDAEVEPYDWCCEGRGGLDSWKSLGYIPVQDFDYKGFGTMTRSISRTLEYAYNDYCIAQIAAALGKSAEKEKYLESSGNWQNLFKEDQTSIWWNGTDTGFTGFFQPRYLNKTWGFQDPLNCSNLDTASVCSLQNTGRETFESSIWEYGFFVPHDQATLISLYGGPAAFVSRLDYLHDSGITYIGNEPSFLTVFQYHYAGRPALSARRAHFYIPAFFSPTPGGLPGNDDSGAMGSFVAFSMMGLFPNPGQNVYLITPPFFESVNITHPLTHRTARIRNVNFDPTYKAIYIQSATLDGKLYTKNWVDHSFFTEGKELVLTLGRNESAWGTKVEDLPPSLGAYEGFTKRSGSLRRTAAPDLWRKTAAFKGQFGSLEESLGM

SEQ ID NO: 30 - RNA, 20 nt, synthetic construct
  source 1..20: mol_type = other RNA
tttagtgcagtattgcatcg

SEQ ID NO: 31 - RNA, 20 nt, synthetic construct
  source 1..20: mol_type = other RNA
ccaatttgtgttcaacaccg

SEQ ID NO: 32 - DNA, 192 bp, synthetic construct
  source 1..192: mol_type = other DNA
tctttgggaaactccagggccaggttgggtgctggagggcgcttccatcttcccctgtgccgtcattgtatgcaaagcagttctgtgtatctaagtagttagcacacccagtgcgggtgagtagtacataaatggtagacattgttagagatggttttggtagcaatagaggacagctcaagtcacctaaat

SEQ ID NO: 33 - RNA, 20 nt, synthetic construct
  source 1..20: mol_type = other RNA
ggaacagaagagagacatcg

SEQ ID NO: 34 - RNA, 20 nt, synthetic construct
  source 1..20: mol_type = other RNA
gtagtggtagagatgagacg

SEQ ID NO: 35 - DNA, 192 bp, synthetic construct
  source 1..192: mol_type = other DNA
cctgcgatccctgcataaccccgcttgaagtgccttatccttgaatgtctccctgcattgttgtctctcccctctgtcgatctctcctctctaagtagttagattgtgaggatgagatgcgttgtttgttgaatggtgagtttggagcttggaaatgatagatagagatggtgatgttgtgtgtacaggaga

SEQ ID NO: 36 - DNA, 1080 bp, Trichoderma reesei
  source 1..1080: mol_type = other DNA
atgaaggcgtccgtctacttggcgtccctgctggcgacgctgtcgatggcggtgcccgtcaaggagctgcagctgcgggccgagccgacggacctgcctcgcctgattgtgtacttccagacgacgcacgacagcagcaaccggcccatctcgatgctgccgctcatcacggagaagggcatcgcgctgacgcacctcattgtgtgctcgttccacatcaaccaaggcggcgtggtgcacctcaacgacttcccgccggacgacccgcacttctacacgctgtggaacgagactatcacgatgaagcaggcgggcgtcaaggtcatgggcatggtgggcggcgcggcgccggggtcctttaacacgcagacgctcgactcgccggactcggccacgtttgagcactactacgggcagctgagggacgccattgtcaacttccagctcgagggcatggacctggacgtcgagcagccgatgagccagcagggcatcgaccggctgattgcgcggctgcgggcggatttcgggcccgacttcctcatcacgctggcgcccgtcgcgtcggcgctcgaggatagcagcaacctgtccggcttcagctacacggcgctgcagcagacgcagggcaacgacattgactggtacaacacgcagttctacagcggcttcggcagcatggcggacacgagcgactacgaccgcatcgtggccaacgggttcgcgcccgccaaggtggtggccggccagctgacgacgcccgagggcgcgggctggatcccgacgagcagcctcaacaacaccattgtctcgctcgtgagcgagtacggccagattggcggcgtcatgggctgggagtacttcaacagcctgcccggcggcaccgcggagccgtgggagtgggcgcagattgtgacggagattctgaggccgggcttggtgccggagctgaagattacggaggacgatgcggcgaggctgacgggtgcgtatgaggagagcgtcaaggcggcggcggcggacaacaagagctttgtgaagaggcctagcattaactattatgctatggtgaatgcttaa